(19) United States
(12) Patent Application Publication
(10) Pub. No.: US 2016/0361352 A1
(43) Pub. Date: Dec. 15, 2016

(54) Treatment of Multiple Sclerosis With Combination of Laquinimod and Glatiramer Acetate

(71) Applicants: Yossi Gilgun, Bat Hefer (IL); Nora Tarcic, Modiin (IL)

(72) Inventors: Yossi Gilgun, Bat Hefer (IL); Nora Tarcic, Modiin (IL)

(73) Assignee: Teva Pharmaceutical Industries, Ltd., Petach-Tikva (IL)

(21) Appl. No.: 15/148,074

(22) Filed: May 6, 2016

Related U.S. Application Data
(63) Continuation of application No. 14/801,595, filed on Jul. 16, 2015, now abandoned.
Application No. 14/801,595 is a continuation of application No. 14/676,973, filed on Apr. 2, 2015, now abandoned.
Application No. 14/676,973 is a continuation of application No. 14/571,101, filed on Dec. 15, 2014, now abandoned.
Application No. 14/571,101 is a continuation of application No. 14/481,359, filed on Sep. 9, 2014, now abandoned.
Application No. 14/481,359 is a continuation of application No. 14/259,950, filed on Apr. 23, 2014, now abandoned.
Application No. 14/259,950 is a continuation of application No. 13/560,851, filed on Jul. 27, 2012, now abandoned.
(60) Provisional application No. 61/512,808, filed on Jul. 28, 2011.

(51) Int. Cl. A61K 31/785; A61K 31/4704; A61K 45/06; A61K 9/08; A61J 1/05; A61K 9/48; A61K 9/20; A61K 9/28; A61K 47/16; A61J 1/03; A61K 9/00; A61K 47/26

(52) U.S. Cl. CPC A61K 31/785; A61K 9/0053; A61K 31/4704; A61K 9/0019; A61K 45/06; A61K 9/0043; A61K 9/0073; A61K 9/08; A61K 47/26; A61K 9/48; A61K 9/20; A61K 9/2866; A61K 47/16; A61J 1/035; A61J 1/05

(57) ABSTRACT

This invention provides a method of treating a patient afflicted with multiple sclerosis or presenting a clinically isolated syndrome comprising administering to the patient laquinimod as an add-on therapy to or in combination with glatiramer acetate. This invention also provides a package and a pharmaceutical composition comprising laquinimod and glatiramer acetate for treating a patient afflicted with multiple sclerosis or presenting a clinically isolated syndrome. This invention also provides laquinimod for use as an add-on therapy or in combination with glatiramer acetate in treating a patient afflicted with multiple sclerosis or presenting a clinically isolated syndrome. This invention further provides use of laquinimod and glatiramer acetate in the preparation of a combination for treating a patient afflicted with multiple sclerosis or presenting a clinically isolated syndrome.

This application claims the benefit of U.S. Provisional Application No. 61/512,808, filed Jul. 28, 2011, the entire content of which is hereby incorporated by reference herein.

Throughout this application, various publications are referred to by first author and year of publication.

Full citations for these publications are presented in a References section immediately before the claims. Disclosures of the documents and publications cited are hereby incorporated by reference in their entireties into this application in order to more fully describe the state of the art as of the date of the invention described herein.

### BACKGROUND

Multiple Sclerosis (MS) is a neurological disease affecting more than 1 million people worldwide. It is the most common cause of neurological disability in young and middle-aged adults and has a major physical, psychological, social and financial impact on subjects and their families, friends and bodies responsible for health care (EMEA Guideline, 2006).

It is generally assumed that MS is mediated by some kind of autoimmune process possibly triggered by infection and superimposed upon a genetic predisposition. It is a chronic inflammatory condition that damages the myelin of the Central Nervous System (CNS). The pathogenesis of MS is characterized by the infiltration of autoreactive T-cells from the circulation directed against myelin antigens into the CNS (Bjartmar, 2002). In addition to the inflammatory phase in MS, axonal loss occurs early in the course of the disease and can be extensive over time, leading to the subsequent development of progressive, permanent, neurologic impairment and, frequently, severe disability (Neuhaus, 2003). Symptoms associated with the disease include fatigue, spasticity, ataxia, weakness, bladder and bowel disturbances, sexual dysfunction, pain, tremor, paroxysmal manifestations, visual impairment, psychological problems and cognitive dysfunction (EMEA Guideline, 2006).

MS disease activity can be monitored by cranial scans, including magnetic resonance imaging (MRI) of the brain, accumulation of disability, as well as rate and severity of relapses. The diagnosis of clinically definite MS as determined by the Poser criteria (Poser, 1983) requires at least two neurological events suggesting demyelination in the CNS separated in time and in location. A clinically isolated syndrome (CIS) is a single monosymptomatic attack suggestive of MS, such as optic neuritis, brain stem symptoms, and partial myelitis. Patients with CIS that experience a second clinical attack are generally considered to have clinically definite multiple sclerosis (CDMS). Over 80 percent of patients with a CIS and MRI lesions go on to develop MS, while approximately 20 percent have a self-limited process (Brex, 2002; Frohman, 2003).

Various MS disease stages and/or types are described in Multiple Sclerosis Therapeutics (Duntiz, 1999). Among them, relapsing-remitting multiple sclerosis (RRMS) is the most common form at the time of initial diagnosis. Many subjects with RRMS have an initial relapsing-remitting course for 5-15 years, which then advances into the secondary progressive MS (SPMS) disease course. Relapses result from inflammation and demyelination, whereas restoration of nerve conduction and remission is accompanied by resolution of inflammation, redistribution of sodium channels on demyelinated axons and remyelination (Neuhaus, 2003; Noseworthy, 2000).

In April 2001, an international panel in association with the National MS Society of America recommended diagnostic criteria for multiple sclerosis. These criteria became known as the McDonald Criteria. The McDonald Criteria make use of MRI techniques and are intended to replace the Poser Criteria and the older Schumacher Criteria (McDonald, 2001). The McDonald Criteria was revised in March 2005 by an international panel (Polman, 2005) and updated again in 2010 (Polman, 2011).

Intervention with disease-modifying therapy at relapsing stages of MS is suggested to reduce and/or prevent accumulating neurodegeneration (Hohlfeld, 2000; De Stefano, 1999). There are currently a number of disease-modifying medications approved for use in relapsing MS (RMS), which includes RRMS and SPMS (The Disease Modifying Drug Brochure, 2006). These include interferon beta 1-a (Avonex® and Rebif®), interferon beta 1-b (Betaseron®), glatiramer acetate (Copaxone®), mitoxantrone (Novantrone®), natalizumab (Tysabri®) and fingolimod (Gilenya®). Most of them are believed to act as immunomodulators. Mitoxantrone and natalizumab are believed to act as immunosuppressants. However, the mechanisms of action of each have been only partly elucidated. Immunosuppressants or cytotoxic agents are used in some subjects after failure of conventional therapies. However, the relationship between changes of the immune response induced by these agents and the clinical efficacy in MS is far from settled (EMEA Guideline, 2006).

Other therapeutic approaches include symptomatic treatment which refers to all therapies applied to improve the symptoms caused by the disease (EMEA Guideline, 2006) and treatment of acute relapses with corticosteroids. While steroids do not affect the course of MS over time, they can reduce the duration and severity of attacks in some subjects.

Laquinimod

Laquinimod is a novel synthetic compound with high oral bioavailability which has been suggested as an oral formulation for the treatment of Multiple Sclerosis (MS) (Polman, 2005; Sandberg-Wollheim, 2005). Laquinimod and its sodium salt form are described, for example, in U.S. Pat. No. 6,077,851.

The mechanism of action of laquinimod is not fully understood. Animal studies show it causes a Th1 (T helper 1 cell, produces pro-inflammatory cytokines) to Th2 (T helper 2 cell, produces anti-inflammatory cytokines) shift with an anti-inflammatory profile (Yang, 2004; Brück, 2011). Another study demonstrated (mainly via the NFkB pathway) that laquinimod induced suppression of genes related to antigen presentation and corresponding inflammatory pathways (Gurevich, 2010). Other suggested potential mechanisms of action include inhibition of leukocyte migration into the CNS, increase of axonal integrity, modulation of cytokine production, and increase in levels of brain-derived neurotrophic factor (BDNF) (Runström, 2006; Brick, 2011).

Laquinimod showed a favorable safety and tolerability profile in two phase III trials (Results of Phase III BRAVO Trial Reinforce Unique Profile of Laquinimod for Multiple Sclerosis Treatment; Teva Pharma, Active Biotech Post Positive Laquinimod Phase 3 ALLEGRO Results).

Glatiramer Acetate (GA)

Glatiramer acetate (GA), also known as Copolymer-1, has been shown to be effective in treating multiple sclerosis (MS) (Lampert, 1978). Daily subcutaneous injections of glatiramer acetate (20 mg/injection) reduce relapse rates, progression of disability, appearance of new lesions by magnetic resonance imaging (MRI), (Johnson, 1995) and appearance of “black holes” (Filippi, 2001).

COPAXONE® is the brand name for a formulation containing glatiramer acetate as the active ingredient. Glatiramer acetate is approved for reducing the frequency of relapses in relapsing-remitting multiple sclerosis (RRMS). Glatiramer acetate consists of the acetate salts of synthetic polypeptides containing four naturally occurring amino acids: L-glutamic acid, L-alanine, L-tyrosine, and L-lysine with an average molar fraction in COPAXONE® of 0.141, 0.427, 0.095 and 0.338, respectively. In COPAXONE®, the average molecular weight of the glatiramer acetate is 4,700-11,000 daltons. Chemically, glatiramer acetate is designated L-glutamic acid polymer with L-alanine, L-lysine and L-tyrosine, acetate (salt). Its structural formula is:

(Glu,Ala,Lys,Tyr)ₓACH₃COOH (C₅H₉NO₄AC₃H₇NO₂AC₆H₁₄N₂O₂AC₉H₁₁NO₃)_{P}APC₂H₄O₂ CAS-147245-92-9.

The recommended dosing schedule of COPAXONE® for relapsing-remitting multiple sclerosis is 20 mg per day injected subcutaneously (Physician's Desk Reference; see also U.S. Pat. Nos. 3,849,550; 5,800,808; 5,858,964, 5,981,589; 6,048,898; 6,054,430; 6,214,791; 6,342,476; and 6,362,161, all of which are hereby incorporated by reference.

Although its mechanism of action is not completely elucidated, GA is thought to bind to and to be displayed as an antigen within the groove of a major histocompatibility complex (MHC) molecule. Alternatively, GA is thought be engulfed by antigen presenting cells (APC) and fragments are then presented. Either way, the presentation of GA leads to the generation of GA-specific T cells. Through mechanisms that are still unclear, the GA-specific T cells are predominantly T helper 2 (Th2) biased. Th2 cells produce Th2 cytokines which inhibit the production of cytokines by Th1 cells or macrophages, and tend to be anti-inflammatory. Unlike interferon-β which apparently has potent activity at the blood-brain barrier (BBB) and impairs the trafficking of inflammatory cells into the CNS, GA has negligible effect at the BBB, allowing GA-specific Th2 lymphocytes to enter the CNS to decrease inflammation through bystander suppression (Yong, 2002).

Add-On/Combination Therapy

The effects of add-on or combination therapy using laquinimod and glatiramer acetate on MS patients have not been reported.

The administration of two drugs to treat a given condition, such as multiple sclerosis, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 1999). In one example, combined administration of GA and interferon (IFN) has been experimentally shown to abrogate the clinical effectiveness of either therapy. (Brod 2000) In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side profile of each drug. In one example, the combination of natalizumab and interferon β-1a was observed to increase the risk of unanticipated side effects. (Vollmer, 2008; Rudick 2006; Kleinschmidt-DeMasters, 2005; Langer-Gould 2005)

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry, 1999).

Therefore, the state of the art at the time of filing is that the effects of an add-on or combination therapy of two drugs, in particular laquinimod and GA, cannot be predicted until the results of a formal combination study are available.

### SUMMARY OF THE INVENTION

This invention provides a method of treating a human patient afflicted with multiple sclerosis or presenting a clinically isolated syndrome comprising orally administering to the patient a daily dose of 0.6 mg laquinimod, and subcutaneously injecting the patient with a daily dose of 20 mg glatiramer acetate, wherein the amounts when taken together is more effective to treat the human patient than when each agent is administered alone.

This invention also provides a method of treating a human patient afflicted with multiple sclerosis or presenting a clinically isolated syndrome comprising periodically administering to the patient an amount of laquinimod and an amount of glatiramer acetate, wherein the amounts when taken together are effective to treat the human patient.

This invention also provides a method of treating a human patient afflicted with an immune disease, comprising periodically administering to the patient an amount of laquinimod and an amount of glatiramer acetate (GA), wherein the amounts when taken together are effective to treat the human patient, and wherein the immune disease is an autoimmune disease, an arthritic condition, a demyelinating disease, an inflammatory disease, multiple sclerosis, relapsing-remitting multiple sclerosis, diabetes mellitus, psoriasis, rheumatoid arthritis, inflammatory bowel disease, Crohn's disease, or systemic lupus erythematosus.

This invention also provides a package comprising a) a first pharmaceutical composition comprising an amount of laquinimod and a pharmaceutically acceptable carrier; b) a second pharmaceutical composition comprising an amount of glatiramer acetate and a pharmaceutically acceptable carrier, and c) instructions for use of the first and second pharmaceutical compositions together to treat a human patient afflicted with relapsing multiple sclerosis or presenting a clinically isolated syndrome.

This invention also provides laquinimod for use as an add-on therapy or in combination with glatiramer acetate in treating a human patient afflicted with multiple sclerosis or presenting a clinically isolated syndrome.

This invention also provides a pharmaceutical composition comprising an amount of laquinimod and an amount of glatiramer acetate for use in treating a human patient afflicted with multiple sclerosis or presenting a clinically isolated syndrome, wherein the laquinimod and the glatiramer acetate are administered simultaneously or contemporaneously.

This invention also provides a pharmaceutical composition comprising an amount of laquinimod and an amount of glatiramer acetate for use in treating a human patient afflicted with an immune disease, wherein the laquinimod and the glatiramer acetate are administered simultaneously or contemporaneously, and wherein the immune disease is an autoimmune disease, an arthritic condition, a demyelinating disease, an inflammatory disease, multiple sclerosis, relapsing-remitting multiple sclerosis, diabetes mellitus, psoriasis, rheumatoid arthritis, inflammatory bowel disease, Crohn's disease, or systemic lupus erythematosus.

This invention also provides a pharmaceutical composition comprising an amount of laquinimod and an amount of glatiramer acetate.

This invention also provides use of an amount of laquinimod and an amount of glatiramer acetate in the preparation of a combination for treating a human patient afflicted with multiple sclerosis or presenting a clinically isolated syndrome wherein the laquinimod and the glatiramer acetate are administered simultaneously or contemporaneously.

This invention also provides pharmaceutical composition comprising an amount of laquinimod for use in treating a subject afflicted with multiple sclerosis or presenting a clinically isolated syndrome as an add-on therapy or in combination with glatiramer acetate by periodically administering the pharmaceutical composition and the glatiramer acetate to the subject.

This invention further provides pharmaceutical composition comprising an amount of glatiramer acetate for use treating a subject afflicted with multiple sclerosis or presenting a clinically isolated syndrome as an add-on therapy or in combination with laquinimod by periodically administering the pharmaceutical composition and the laquinimod to the subject.

### BRIEF DESCRIPTION OF THE DRAWINGS

FIG. 1 is a graphical representation of the experimental results from Example 2.1. FIG. 1A shows the clinical score for the EAE rodents in each group (on the y-axis) against the days after induction of the disease (on the x-axis). Figure B shows the weight for the EAE rodents in each group (on the y-axis) against the days after induction of the disease (on the x-axis).

FIG. 2 is a graphical representation of the experimental results from Example 2.2. The graph shows the clinical score for the EAE rodents in each group (on the y-axis) against the days after induction of the disease (on the x-axis).

FIG. 3 is a graphical representation of the experimental results from Example 2.3. The graph shows the clinical score for the EAE rodents in each group (on the y-axis) against the days after induction of the disease (on the x-axis).

FIG. 4 is a graphical representation summarizing experimental results from Examples 2.1-2.3. FIG. 4A shows the clinical score for the EAE rodents in each group (on the y-axis) against the days after induction of the disease (on the x-axis). FIG. 4B shows the percent inhibition (on the y-axis) for the EAE rodents in each group (on the x-axis).

FIG. 5 is a graphical representation of the experimental results from Example 2.8. The graph shows the daily mean clinical score for the EAE rodents in each group (on the y-axis) against the days of observation (on the x-axis).

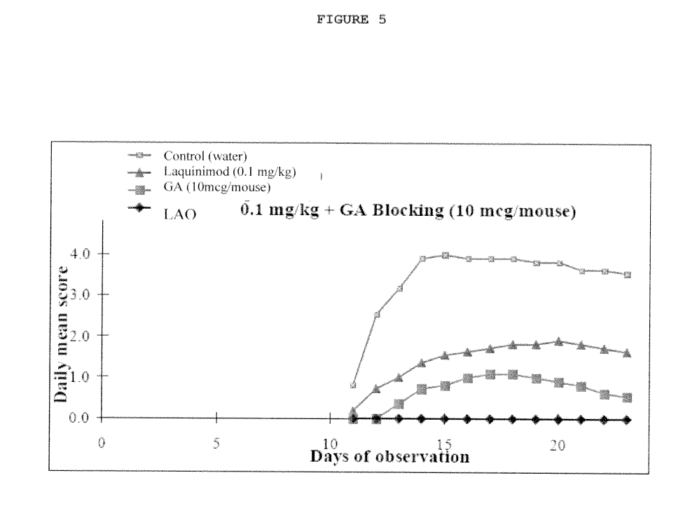

### DETAILED DESCRIPTION OF THE INVENTION

This invention provides a method of treating a human patient afflicted with multiple sclerosis or presenting a clinically isolated syndrome comprising orally administering to the patient a daily dose of 0.6 mg laquinimod or pharmaceutically acceptable salt thereof, and subcutaneously injecting the patient with a daily dose of 20 mg glatiramer acetate, wherein the amounts when taken together is more effective to treat the human patient than when each agent is administered alone.

In one embodiment, the multiple sclerosis is relapsing multiple sclerosis. In another embodiment, the relapsing multiple sclerosis is relapsing-remitting multiple sclerosis.

In one embodiment, the amount of laquinimod and the glatiramer acetate when taken together is effective to reduce a symptom of multiple sclerosis in the human patient. In another embodiment, the symptom is a MRI-monitored multiple sclerosis disease activity, relapse rate, accumulation of physical disability, frequency of relapses, frequency of clinical exacerbation, brain atrophy, risk for confirmed progression, or time to confirmed disease progression.

In one embodiment, the accumulation of physical disability is assessed by the time to confirmed disease progression as measured by Kurtzke Expanded Disability Status Scale (EDSS) score. In another embodiment, the patient had an EDSS score of 0-5 prior to administration of laquinimod. In another embodiment, the patient had an EDSS score of 1-5.5 prior to administration of laquinimod. In another embodiment, the patient had an EDSS score of 0-5.5 prior to administration of laquinimod. In another embodiment, the patient had an EDSS score of 5.5 or greater prior to administration of laquinimod. In another embodiment, confirmed disease progression is a 1 point increase of the EDSS score. In yet another embodiment, confirmed disease progression is a 0.5 point increase of the EDSS score.

In one embodiment, time to confirmed disease progression is increased by 10-100%. In another embodiment, time to confirmed disease progression is increased by 20-80%. In another embodiment, time to confirmed disease progression is increased by 20-60%. In another embodiment, time to confirmed disease progression is increased by 30-50%. In yet another embodiment, time to confirmed disease progression is increased by at least 50%.

In one embodiment, laquinimod is laquinimod sodium.

In one embodiment, the patient is injected subcutaneously with 0.5 ml of an aqueous pharmaceutical solution which contains in solution 20 mg glatiramer acetate and 20 mg mannitol. In another embodiment, the patient is injected subcutaneously with 1.0 ml of an aqueous pharmaceutical solution which contains in solution 20 mg glatiramer acetate and 40 mg mannitol. In one embodiment, the amount of glatiramer acetate administered is suboptimal.

In one embodiment, the glatiramer acetate is administered intramuscularly. In another embodiment, the glatiramer acetate is administered subcutaneously. In another embodiment, the glatiramer acetate is administered 1-5 times a month. In another embodiment, the glatiramer acetate is administered 1-3 times a month. In another embodiment, the glatiramer acetate is administered 1-5 times a week. In another embodiment, the glatiramer acetate is administered 1-3 times a week. In another embodiment, the glatiramer acetate is administered 1-5 times a day. In another embodiment, the glatiramer acetate is administered 1-3 times a day. In another embodiment, the glatiramer acetate is administered every other day. In yet another embodiment, the glatiramer acetate is administered daily

In one embodiment, the administration of laquinimod substantially precedes the administration of glatiramer acetate. In another embodiment, the administration of glatiramer acetate substantially precedes the administration of laquinimod.

In an embodiment, the human patient is receiving glatiramer acetate therapy prior to initiating laquinimod therapy. In another embodiment, the human patient is receiving glatiramer acetate therapy for at least 24 weeks prior to initiating laquinimod therapy. In another embodiment, the human patient is receiving glatiramer acetate therapy for about 24 weeks prior to initiating laquinimod therapy. In another embodiment, the human patient is receiving glatiramer acetate therapy for at least 28 weeks prior to initiating laquinimod therapy. In another embodiment, the human patient is receiving glatiramer acetate therapy for about 28 weeks prior to initiating laquinimod therapy. In another embodiment, the human patient is receiving glatiramer acetate therapy for at least 48 weeks prior to initiating laquinimod therapy. In another embodiment, the human patient is receiving glatiramer acetate therapy for about 48 weeks prior to initiating laquinimod therapy. In another embodiment, the human patient is receiving glatiramer acetate therapy for at least 52 weeks prior to initiating laquinimod therapy. In yet another embodiment, the human patient is receiving glatiramer acetate therapy for about 52 weeks prior to initiating laquinimod therapy.

In one embodiment, the laquinimod is administered in the morning. In another embodiment, the laquinimod is administered at night. In one embodiment, the laquinimod is with food. In another embodiment, the laquinimod is administered without food.

In one embodiment, the glatiramer acetate is administered in the morning. In another embodiment, the glatiramer acetate is administered at night. In one embodiment, the glatiramer acetate is administered with food. In another embodiment, the glatiramer acetate is administered without food.

In one embodiment, the laquinimod is administered simultaneously with the glatiramer acetate. In another embodiment, the laquinimod is administered contemporaneously with the glatiramer acetate. In another embodiment, the laquinimod is administered immediately before or immediately after the glatiramer acetate. In another embodiment, the laquinimod is administered within 1 hour before or after the glatiramer acetate. In another embodiment, the laquinimod is administered within 3 hour before or after the glatiramer acetate. In another embodiment, the laquinimod is administered within 6 hour before or after the glatiramer acetate. In another embodiment, the laquinimod is administered within 12 hour before or after the glatiramer acetate. In another embodiment, the laquinimod is administered within 24 hour before or after the glatiramer acetate.

In one embodiment, the method further comprises administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies.

In an embodiment, the periodic administration of laquinimod and glatiramer acetate continues for more than 30 days. In another embodiment, the periodic administration of laquinimod and glatiramer acetate continues for more than 42 days. In yet another embodiment, the periodic administration of laquinimod and glatiramer acetate continues for 6 months or more.

In one embodiment, the administration of laquinimod and glatiramer acetate inhibits a symptom of relapsing multiple sclerosis by at least 20%. In another embodiment, the administration of laquinimod and glatiramer acetate inhibits a symptom of relapsing multiple sclerosis by at least 30%. In another embodiment, the administration of laquinimod and glatiramer acetate inhibits a symptom of relapsing multiple sclerosis by at least 40%. In another embodiment, the administration of laquinimod and glatiramer acetate inhibits a symptom of relapsing multiple sclerosis by at least 50%. In another embodiment, the administration of laquinimod and glatiramer acetate inhibits a symptom of relapsing multiple sclerosis by more than 100%. In another embodiment, the administration of laquinimod and glatiramer acetate inhibits a symptom of relapsing multiple sclerosis by more than 300%. In yet another embodiment, the administration of laquinimod and glatiramer acetate inhibits a symptom of relapsing multiple sclerosis by more than 1000%.

In one embodiment, each of the amount of laquinimod when taken alone, and the amount of glatiramer acetate when taken alone is effective to treat the human patient. In another embodiment, either the amount of laquinimod when taken alone, the amount of glatiramer acetate when taken alone, or each such amount when taken alone is not effective to treat the human patient.

This invention also provides a method of treating a human patient afflicted with multiple sclerosis or presenting a clinically isolated syndrome comprising periodically administering to the patient an amount of laquinimod and an amount of glatiramer acetate, wherein the amounts when taken together are effective to treat the human patient. In one embodiment, the amount of laquinimod and the amount of glatiramer acetate when taken together is more effective to treat the human patient than when each agent is administered along.

In one embodiment, the multiple sclerosis is relapsing multiple sclerosis. In another embodiment, the relapsing multiple sclerosis is relapsing-remitting multiple sclerosis.

In one embodiment, the amount of laquinimod and the amount of glatiramer acetate when taken together is effective to reduce a symptom of multiple sclerosis in the human patient. In another embodiment, the symptom is a MRI-monitored multiple sclerosis disease activity, relapse rate, accumulation of physical disability, frequency of relapses, decreased time to confirmed disease progression, decreased time to confirmed relapse, frequency of clinical exacerbation, brain atrophy, neuronal dysfunction, neuronal injury, neuronal degeneration, neuronal apoptosis, risk for confirmed progression, visual function, fatigue, impaired mobility, cognitive impairment, reduction of brain volume, abnormalities observed in whole Brain MTR histogram, deterioration in general health status, functional status, quality of life, and/or symptom severity on work.

In one embodiment, the amount of laquinimod and the amount of glatiramer acetate when taken together is effective to decrease or inhibit reduction of brain volume. In another embodiment, brain volume is measured by percent brain volume change (PBVC).

In one embodiment, the amount of laquinimod and the amount of glatiramer acetate when taken together is effective to increase time to confirmed disease progression. In another embodiment, time to confirmed disease progression is increased by 20-60%. In yet another embodiment, time to confirmed disease progression is increased by at least 50%.

In one embodiment, the amount of laquinimod and the amount of glatiramer acetate when taken together is effective to decrease abnormalities observed in whole Brain MTR histogram. In another embodiment, the accumulation of physical disability is measured by Kurtzke Expanded Disability Status Scale (EDSS) score. In another embodiment, the accumulation of physical disability is assessed by the time to confirmed disease progression as measured by Kurtzke Expanded Disability Status Scale (EDSS) score. In another embodiment, the patient had an EDSS score of 0-5.5 prior to administration of laquinimod. In another embodiment, the patient had an EDSS score of 1.5-4.5 prior to administration of laquinimod. In another embodiment, the patient had an EDSS score of 5.5 or greater prior to administration of laquinimod. In another embodiment, confirmed disease progression is a 1 point increase of the EDSS score. In another embodiment, confirmed disease progression is a 0.5 point increase of the EDSS score.

In one embodiment, impaired mobility is assessed by the Timed-25 Foot Walk test. In another embodiment, impaired mobility is assessed by the 12-Item Multiple Sclerosis Walking Scale (MSWS-12) self-report questionnaire. In another embodiment, impaired mobility is assessed by the Ambulation Index (AI). In another embodiment, impaired mobility is assessed by the Six-Minute Walk (6MW) Test. In another embodiment, impaired mobility is assessed by the Lower Extremity Manual Muscle Test (LEMMT) Test

In one embodiment, the amount of laquinimod and the amount of glatiramer acetate when taken together is effective to reduce cognitive impairment. In another embodiment, cognitive impairment is assessed by the Symbol Digit Modalities Test (SDMT) score.

In one embodiment, general health status is assessed by the EuroQoL (EQ5D) questionnaire, Subject Global Impression (SGI) or Clinician Global Impression of Change (CGIC). In another embodiment, functional status is measured by the patient's Short-Form General Health survey (SF-36) Subject Reported Questionnaire score. In another embodiment, quality of life is assessed by SF-36, EQ5D, Subject Global Impression (SGI) or Clinician Global Impression of Change (CGIC). In another embodiment, the patient's SF-36 mental component summary score (MSC) is improved. In another embodiment, the patient's SF-36 physical component summary sore (PSC) is improved.

In one embodiment, fatigue is assessed by the EQSD, the patient's Modified Fatigue Impact Scale (MFIS) score or the French valid versions of the Fatigue Impact Scale (EMIF-SEP) score. In another embodiment, symptom severity on work is measured by the work productivity and activities impairment General Health (WPAI-GH) questionnaire.

In an embodiment, laquinimod is laquinimod sodium. In another embodiment, laquinimod is administered via oral administration. In another embodiment, laquinimod is administered daily. In another embodiment, laquinimod is administered more often than once daily. In another embodiment, laquinimod is administered less often than once daily.

In one embodiment, the amount laquinimod administered is less than 0.6 mg/day. In another embodiment, the amount laquinimod administered is 0.1-40.0 mg/day. In another embodiment, the amount laquinimod administered is 0.1-2.5 mg/day. In another embodiment, the amount laquinimod administered is 0.25-2.0 mg/day. In another embodiment, the amount laquinimod administered is 0.5-1.2 mg/day. In another embodiment, the amount laquinimod administered is 0.25 mg/day. In another embodiment, the amount laquinimod administered is 0.3 mg/day. In another embodiment, the amount laquinimod administered is 0.5 mg/day. In another embodiment, the amount laquinimod administered is 0.6 mg/day. In another embodiment, the amount laquinimod administered is 1.0 mg/day. In another embodiment, the amount laquinimod administered is 1.2 mg/day. In another embodiment, the amount laquinimod administered is 1.5 mg/day. In yet another embodiment, the amount laquinimod administered is 2.0 mg/day.

In one embodiment, the amount glatiramer acetate administered is 0.1-1000 mg/day. In another embodiment, the amount glatiramer acetate administered is 50-150 ms/day. In another embodiment, the amount glatiramer acetate administered is 0.1-70 mg/day. In another embodiment, the amount glatiramer acetate administered is 10-80 mg/day. In another embodiment, the amount glatiramer acetate administered is 1 mg/day. In another embodiment, the amount glatiramer acetate administered is 5 mg/day. In another embodiment, the amount glatiramer acetate administered is 15 mg/day. In another embodiment, the amount glatiramer acetate administered is 20 mg/day. In another embodiment, the amount glatiramer acetate administered is 30 mg/day. In another embodiment, the amount glatiramer acetate administered is 40 mg/day. In another embodiment, the amount glatiramer acetate administered is 50 mg/day. In another embodiment, the amount glatiramer acetate administered is 100 mg/day. In another embodiment, the amount glatiramer acetate administered is 10-600 mg/week. In another embodiment, the amount glatiramer acetate administered is 300 mg/week.

In one embodiment, administration of glatiramer acetate is effected daily. In another embodiment, administration of glatiramer acetate is effected twice a day at half the amount. In another embodiment, administration of glatiramer acetate is effected once every 5 to 9 days.

In one embodiment, glatiramer acetate is administered orally. In another embodiment, glatiramer acetate is administered nasally. In another embodiment, glatiramer acetate is inhaled. In another embodiment, glatiramer acetate is administered by subcutaneous injection. In another embodiment, glatiramer acetate is administered over a period of seven days with at least one day between every subcutaneous injection. In another embodiment, glatiramer acetate is administered through an intravenous, intraperitoneal, intramuscular, intranasal, buccal, vaginal, rectal, intraocular, intrathecal, topical or intradermal route.

In one embodiment, the patient is injected subcutaneously with 0.5 ml of an aqueous pharmaceutical solution which contains in solution 20 mg glatiramer acetate and 20 mg mannitol. In another embodiment, a loading dose of an amount different form the intended dose is administered for a period of time at the start of the periodic administration. In another embodiment, the loading dose is double the amount of the intended dose. In another embodiment, the loading dose administered for two days at the start of the periodic administration.

In one embodiment, the administration of laquinimod substantially precedes the administration of glatiramer acetate. In another embodiment, the administration of glatiramer acetate substantially precedes the administration of laquinimod.

In one embodiment, the human patient is receiving glatiramer acetate therapy prior to initiating laquinimod therapy. In another embodiment, the human patient is receiving glatiramer acetate therapy for at least 24 weeks prior to initiating laquinimod therapy. In another embodiment, the human patient is receiving glatiramer acetate therapy for at least 28 weeks prior to initiating laquinimod therapy. In another embodiment, the human patient is receiving glatiramer acetate therapy for at least 48 weeks prior to initiating laquinimod therapy. In another embodiment, the human patient is receiving glatiramer acetate therapy for at least 52 weeks prior to initiating laquinimod therapy.

In an embodiment, the method further comprises administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies.

In one embodiment, the periodic administration of laquinimod and glatiramer acetate continues for at least 3 days. In another embodiment, the periodic administration of laquinimod and glatiramer acetate continues for more than 30 days. In another embodiment, the periodic administration of laquinimod and glatiramer acetate continues for more than 42 days. In another embodiment, the periodic administration of laquinimod and glatiramer acetate continues for 8 weeks or more. In another embodiment, the periodic administration of laquinimod and glatiramer acetate continues for at least 12 weeks. In another embodiment, the periodic administration of laquinimod and glatiramer acetate continues for at least 24 weeks. In another embodiment, the periodic administration of laquinimod and glatiramer acetate continues for more than 24 weeks. In another embodiment, the periodic administration of laquinimod and glatiramer acetate continues for 6 months or more.

In one embodiment, the administration of laquinimod and glatiramer acetate inhibits a symptom of relapsing multiple sclerosis by at least 20%. In another embodiment, the administration of laquinimod and glatiramer acetate inhibits a symptom of relapsing multiple sclerosis by at least 30%. In another embodiment, the administration of laquinimod and glatiramer acetate inhibits a symptom of relapsing multiple sclerosis by at least 50%. In another embodiment, the administration of laquinimod and glatiramer acetate inhibits a symptom of relapsing multiple sclerosis by at least 70%. In another embodiment, the administration of laquinimod and glatiramer acetate inhibits a symptom of relapsing multiple sclerosis by more than 100%. In another embodiment, the administration of laquinimod and glatiramer acetate inhibits a symptom of relapsing multiple sclerosis by more than 300%. In another embodiment, the administration of laquinimod and glatiramer acetate inhibits a symptom of relapsing multiple sclerosis by more than 1000%.

In one embodiment, each of the amount of laquinimod when taken alone, and the amount of glatiramer acetate when taken alone is effective to treat the human patient. In another embodiment, either the amount of laquinimod when taken alone, the amount of glatiramer acetate when taken alone, or each such amount when taken alone is not effective to treat the human patient.

In one embodiment, the patient has been identified as a responder to glatiramer treatment. In another embodiment, the patient has been identified as a non-responder to glatiramer treatment.

This invention also provides a method of treating a human patient afflicted with an immune disease, comprising periodically administering to the patient an amount of laquinimod and an amount of glatiramer acetate (GA), wherein the amounts when taken together are effective to treat the human patient, and wherein the immune disease is an autoimmune disease, an arthritic condition, a demyelinating disease, an inflammatory disease, multiple sclerosis, relapsing-remitting multiple sclerosis, diabetes mellitus, psoriasis, rheumatoid arthritis, inflammatory bowel disease, Crohn's disease, or systemic lupus erythematosus.

This invention also provides a package comprising a) a first pharmaceutical composition comprising an amount of laquinimod and a pharmaceutically acceptable carrier; b) a second pharmaceutical composition comprising an amount of glatiramer acetate and a pharmaceutically acceptable carrier, and c) instructions for use of the first and second pharmaceutical compositions together to treat a human patient afflicted with relapsing multiple sclerosis or presenting a clinically isolated syndrome.

In one embodiment, the first pharmaceutical composition is in the form of an aerosol or inhalable powder. In another embodiment, the first pharmaceutical composition is in liquid form. In another embodiment, the first pharmaceutical composition is in solid form. In another embodiment, the first pharmaceutical composition is in capsule form. In another embodiment, the first pharmaceutical composition is in tablet form. In another embodiment, the tablets are coated with a coating which inhibits oxygen from contacting the core. In yet another embodiment, the coating comprises a cellulosic polymer, a detackifier, a gloss enhancer, and pigment.

In one embodiment, the first pharmaceutical composition further comprises mannitol. In another embodiment, the first pharmaceutical composition further comprises an alkalinizing agent. In another embodiment, the alkalinizing agent is meglumine. In another embodiment, the first pharmaceutical composition further comprises an oxidation reducing agent.

In an embodiment, the first pharmaceutical composition is stable and free of an alkalinizing agent or an oxidation reducing agent. In another embodiment, the first pharmaceutical composition is free of an alkalinizing agent and free of an oxidation reducing agent.

In one embodiment, the first pharmaceutical composition is stable and free of disintegrant. In another embodiment, the first pharmaceutical composition further comprises a lubricant. In another embodiment, the lubricant is present in the composition as solid particles. In another embodiment, the lubricant is sodium stearyl fumarate or magnesium stearate.

In an embodiment, the first pharmaceutical composition further comprises a filler. In another embodiment, the filler is present in the composition as solid particles. In another embodiment, the filler is lactose, lactose monohydrate, starch, isomalt, mannitol, sodium starch glycolate, sorbitol, lactose spray dried, lactose anhydrous, or a combination thereof. In another embodiment, the filler is mannitol or lactose monohydrate.

In one embodiment, the package further comprises a desiccant. In another embodiment, the desiccant is silica gel.

In one embodiment, the first pharmaceutical composition is stable has a moisture content of no more than 4%. In another embodiment, laquinimod is present in the composition as solid particles.

In one embodiment, the package is a sealed packaging having a moisture permeability of not more than 15 mg/day per liter. In another embodiment, the sealed package is a blister pack in which the maximum moisture permeability is no more than 0.005 mg/day. In another embodiment, the sealed package is a bottle. In another embodiment, the bottle is closed with a heat induction liner. In another embodiment, the sealed package comprises an HDPE bottle. In another embodiment, the sealed package comprises an oxygen absorbing agent. In another embodiment, the oxygen absorbing agent is iron.

In one embodiment, the amount of laquinimod in the first composition is less than 0.6 mg. In another embodiment, the amount of laquinimod in the composition is 0.1-40.0 mg. In another embodiment, the amount of laquinimod in the first composition is 0.1-2.5 mg. In another embodiment, the amount of laquinimod in the first composition is 0.25-2.0 mg. In another embodiment, the amount of laquinimod in the first composition is 0.5-1.2 mg. In another embodiment, the amount of laquinimod in the first composition is 0.25 mg. In another embodiment, the amount of laquinimod in the first composition is 0.3 mg. In another embodiment, the amount of laquinimod in the first composition is 0.5 mg. In another embodiment, the amount of laquinimod in the first composition is 0.6 mg. In another embodiment, the amount of laquinimod in the first composition is 1.0 mg. In another embodiment, the amount of laquinimod in the first composition is 12 mg. In another embodiment, the amount of laquinimod in the first composition is 1.5 mg. In another embodiment, the amount of laquinimod in the first composition is 2.0 mg.

In an embodiment, the amount glatiramer acetate in the second composition is 0.1-1000 mg. In another embodiment, the amount glatiramer acetate in the second composition is 50-150 mg. In another embodiment, the amount glatiramer acetate in the second composition is 10-600 mg. In another embodiment, the amount glatiramer acetate in the second composition is 0.1-70 mg. In another embodiment, the amount glatiramer acetate in the second composition is 10-80 mg. In another embodiment, the amount glatiramer acetate in the second composition is 1 mg. In another embodiment, the amount glatiramer acetate in the second composition is 5 mg. In another embodiment, the amount glatiramer acetate in the second composition is 15 mg. In another embodiment, the amount glatiramer acetate in the second composition is 20 mg. In another embodiment, the amount glatiramer acetate in the second composition is 30 mg. In another embodiment, the amount glatiramer acetate in the second composition is 40 mg. In another embodiment, the amount glatiramer acetate in the second composition is 50 mg. In another embodiment, the amount glatiramer acetate in the second composition is 100 mg. In another embodiment, the amount glatiramer acetate in the second composition is 300 mg.

In one embodiment, the second composition is a unit dose of 0.5 ml aqueous solution comprising 20 mg of glatiramer acetate. In another embodiment, the second composition is a unit dose of 0.5 ml aqueous solution comprising 20 mg of glatiramer acetate and 20 mg mannitol. In another embodiment, the second composition is a unit dose of 1 ml aqueous pharmaceutical solution which contains in solution 20 mg glatiramer acetate and 40 mg mannitol. In another embodiment, the second composition is a unit dose of 1 ml aqueous pharmaceutical solution which contains in solution 40 mg glatiramer acetate. In another embodiment, the second composition is in an enterically-coated form.

This invention also provides laquinimod for use as an add-on therapy or in combination with glatiramer acetate in treating a human patient afflicted with multiple sclerosis or presenting a clinically isolated syndrome.

This invention also provides a pharmaceutical composition comprising an amount of laquinimod and an amount of glatiramer acetate for use in treating a human patient afflicted with multiple sclerosis or presenting a clinically isolated syndrome, wherein the laquinimod and the glatiramer acetate are administered simultaneously or contemporaneously.

This invention also provides a pharmaceutical composition comprising an amount of laquinimod and an amount of glatiramer acetate for use in treating a human patient afflicted with an immune disease, wherein the laquinimod and the glatiramer acetate are administered simultaneously or contemporaneously, and wherein the immune disease is an autoimmune disease, an arthritic condition, a demyelinating disease, an inflammatory disease, multiple sclerosis, relapsing-remitting multiple sclerosis, diabetes mellitus, psoriasis, rheumatoid arthritis, inflammatory bowel disease, Crohn's disease, or systemic lupus erythematosus.

This invention also provides a pharmaceutical composition comprising an amount of laquinimod and an amount of glatiramer acetate.

In an embodiment, the pharmaceutical composition is in the form of an aerosol or inhalable powder. In another embodiment, the pharmaceutical composition is in liquid form. In another embodiment, the pharmaceutical composition is in solid form. In another embodiment, the pharmaceutical composition is in capsule form. In another embodiment, the pharmaceutical composition is in tablet form. In another embodiment, the tablets are coated with a coating which inhibits oxygen from contacting the core. In another embodiment, the coating comprises a cellulosic polymer, a detackifier, a gloss enhancer, and pigment.

In an embodiment, the pharmaceutical composition further comprises mannitol. In another embodiment, the pharmaceutical composition further comprises an alkalinizing agent. In an embodiment, the alkalinizing agent is meglumine. In another embodiment, the pharmaceutical composition further comprises an oxidation reducing agent.

In one embodiment, the pharmaceutical composition is free of an alkalinizing agent or an oxidation reducing agent. In another embodiment, the pharmaceutical composition is free of an alkalinizing agent and free of an oxidation reducing agent.

In an embodiment, the pharmaceutical composition is stable and free of disintegrant. In another embodiment, the pharmaceutical composition further comprises a lubricant. In one embodiment, the lubricant is present in the composition as solid particles. In another embodiment, the lubricant is sodium stearyl fumarate or magnesium stearate.

In an embodiment, the pharmaceutical composition further comprises a filler. In another embodiment, the filler is present in the composition as solid particles. In another embodiment, the filler is lactose, lactose monohydrate, starch, isomalt, mannitol, sodium starch glycolate, sorbitol, lactose spray dried, lactose anhydrous, or a combination thereof. In another embodiment, the filler is mannitol or lactose monohydrate.

In one embodiment, the amount of laquinimod in the composition is less than 0.6 mg. In another embodiment, the amount of laquinimod in the composition is 0.1-40.0 mg. In another embodiment, the amount of laquinimod in the composition is 0.1-2.5 mg. In another embodiment, the amount of laquinimod in the composition is 0.25-2.0 mg. In another embodiment, the amount of laquinimod in the composition is 0.5-1.2 mg. In another embodiment, the amount of laquinimod in the composition is 0.25 mg. In another embodiment, the amount of laquinimod in the composition is 03 mg. In another embodiment, the amount of laquinimod in the composition is 0.5 mg. In another embodiment, the amount of laquinimod in the composition is 0.6 mg. In another embodiment, the amount of laquinimod in the composition is 1.0 mg. In another embodiment, the amount of laquinimod in the composition is 1.2 mg. In another embodiment, the amount of laquinimod in the composition is 1.5 mg. In another embodiment, the amount of laquinimod in the composition is 2.0 mg.

In one embodiment, the amount glatiramer acetate in the composition is 0.1-1000 mg. In another embodiment, the amount glatiramer acetate in the composition is 50-150 mg. In another embodiment, the amount glatiramer acetate in the composition is 10-600 mg. In another embodiment, the amount glatiramer acetate in the composition is 0.1-70 mg. In another embodiment, the amount glatiramer acetate in the composition is 10-80 mg. In another embodiment, the amount glatiramer acetate in the composition is 1 mg. In another embodiment, the amount glatiramer acetate in the composition is 5 mg. In another embodiment, the amount glatiramer acetate in the composition is 15 mg. In another embodiment, the amount glatiramer acetate in the composition is 20 mg. In another embodiment, n the amount glatiramer acetate in the composition is 30 mg. In another embodiment, the amount glatiramer acetate in the composition is 40 mg. In another embodiment, the amount glatiramer acetate in the composition is 50 mg. In another embodiment, the amount glatiramer acetate in the composition is 100 mg. In yet another embodiment, the amount glatiramer acetate in the composition is 300 mg.

In one embodiment, the composition is a unit dose of 0.5 ml aqueous solution comprising 20 mg of glatiramer acetate. In another embodiment, the composition is a unit dose of 0.5 ml aqueous solution comprising 20 mg of glatiramer acetate and 20 mg mannitol. In another embodiment, the composition is a unit dose of 1 ml aqueous pharmaceutical solution which contains in solution 20 mg glatiramer acetate and 40 mg mannitol. In another embodiment, the composition is a unit dose of 1 ml aqueous pharmaceutical solution which contains in solution 40 mg glatiramer acetate.

This invention further provides use of an amount of laquinimod and an amount of glatiramer acetate in the preparation of a combination for treating a human patient afflicted with multiple sclerosis or presenting a clinically isolated syndrome wherein the laquinimod and the glatiramer acetate are administered simultaneously or contemporaneously.

Disclosed also is a method of treating a human patient afflicted with multiple sclerosis or presenting a clinically isolated syndrome comprising orally administering to the patient a daily dose of 0.6 mg laquinimod, and subcutaneously injecting the patient with a daily dose of 20 mg glatiramer acetate, wherein the amounts when taken together is more effective to treat the human patient than when each agent is administered alone.

Disclosed also is a method of treating a human patient afflicted with multiple sclerosis or a patient presenting a clinically isolated syndrome suggestive of multiple sclerosis comprising periodically administering to the human patient an amount of laquinimod and an amount of glatiramer acetate, wherein the amounts when taken together is more effective to treat the human patient than when each agent is administered alone. In one embodiment, the patient has experienced a single clinical attack suggestive of multiple sclerosis and has had at least one lesion suggestive of multiple sclerosis. In another embodiment, the amount of laquinimod and the amount of glatiramer acetate when taken together is more effective to delaying the conversion to clinically definite multiple sclerosis in the patient than when each agent is administered alone.

This invention also provides pharmaceutical composition comprising an amount of laquinimod for use in treating a subject afflicted with multiple sclerosis or presenting a clinically isolated syndrome as an add-on therapy or in combination with glatiramer acetate by periodically administering the pharmaceutical composition and the glatiramer acetate to the subject.

This invention further provides pharmaceutical composition comprising an amount of glatiramer acetate for use treating a subject afflicted with multiple sclerosis or presenting a clinically isolated syndrome as an add-on therapy or in combination with laquinimod by periodically administering the pharmaceutical composition and the laquinimod to the subject.

For the foregoing embodiments, each embodiment disclosed herein is contemplated as being applicable to each of the other disclosed embodiments. In addition, the elements recited in the packaging and pharmaceutical composition embodiments can be used in the method embodiments described herein.

Glatiramer Acetate

Glatiramer acetate mixtures, compositions, the process for the manufacture thereof, the use thereof for treatment of various conditions, and the corresponding dosages and regimens are described in, e.g., PCT International Application Publication Nos. WO 1998/30227, WO 2000/05250, WO 2000/18794, WO 2004/103297, WO 2006/029393, WO 2006/029411, WO 2006/083608, WO 2006/089164, WO 2006/116602. WO 2009/070298, WO 2011/022063, WO 2012/051106, WO 2003/048735, and WO 2011/008274, U.S. Patent Application Publication Nos. 2011-0230413 and 2008-027526, and U.S. Pat. Nos. 8,008,258 and 7,556,767, each of which is hereby incorporated by reference in its entireties into this application.

Laquinimod

Laquinimod mixtures, compositions, and the process for the manufacture thereof are described in, e.g., U.S. Pat. No. 6,077,851, U.S. Pat. No. 7,884,208, U.S. Pat. No. 7,989,473, U.S. Pat. No. 8,178,127, U.S. Application Publication No. 2010-0055072, U.S. Application Publication No. 2012-0010238, and U.S. Application Publication No. 2012-0010239, each of which is hereby incorporated by reference in its entireties into this application.

Use of laquinimod for treatment of various conditions, and the corresponding dosages and regimens, are described in U.S. Pat. No. 6,077,851 (multiple sclerosis, insulin-dependent diabetes mellitus, systemic lupus erythematosus, rheumatoid arthritis, inflammatory bowel disease, psoriasis, inflammatory respiratory disorder, atherosclerosis, stroke, and Alzheimer's disease), U.S. Application Publication No. 2011-0027219 (Crohn's disease), U.S. Application Publication No. 2010-0322900 (Relapsing-remitting multiple sclerosis), U.S. Application Publication No. 2011-0034508 (brain-derived neurotrophic factor (BDNF)-related diseases), U.S. Application Publication No. 2011-0218179 (active lupus nephritis), U.S. Application Publication No. 2011-0218203 (rheumatoid arthritis), U.S. Application Publication No. 2011-0217295 (active lupus arthritis), and U.S. Application Publication No. 2012-0142730 (reducing fatigue, improving quality of life, and providing neuroprotection in MS patients), each of which is hereby incorporated by reference in its entireties into this application.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/074899, which are hereby incorporated by reference into this application.

Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar. Capsule or tablets can be easily formulated and can be made easy to swallow or chew; other solid forms include granules, and bulk powders.

Tablets may contain suitable binders, lubricants, disintegrating agents (disintegrants), coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethykellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators (disintegrants) include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Pat. No. 7,589,208, PCT International Application Publication Nos. WO 2005/074899, WO 2007/047863, and 2007/146248.

General techniques and compositions for making dosage forms useful in the present invention are described-in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds).; Modern Pharmaceutics Drugs and the Pharmaceutical Sciences, Vol. 40 (Gilbert S. Banker, Christopher T. Rhodes, Eds). These references in their entireties are hereby incorporated by reference into this application.

Disclosed is a method for treating a subject afflicted with relapsing multiple sclerosis using laquinimod with glatiramer acetate which provides a more efficacious treatment than each agent alone. The use of laquinimod for relapsing multiple sclerosis had been previously suggested in, e.g., U.S. Pat. No. 6,077,851. However, the inventors have surprisingly found that the combination of laquinimod and glatiramer acetate (GA) is particularly effective for the treatment of relapsing multiple sclerosis as compared to each agent alone.

Terms

As used herein, and unless stated otherwise, each of the following terms shall have the definition set forth below.

As used herein, “laquinimod” means laquinimod acid or a pharmaceutically acceptable salt thereof

As used herein, an “amount” or “dose” of laquinimod as measured in milligrams refers to the milligrams of laquinimod acid present in a preparation, regardless of the form of the preparation. A “dose of 0.6 mg laquinimod” means the amount of laquinimod acid in a preparation is 0.6 mg, regardless of the form of the preparation. Thus, when in the form of a salt, e.g. a laquinimod sodium salt, the weight of the salt form necessary to provide a dose of 0.6 mg laquinimod would be greater than 0.6 mg (e.g., 0.64 mg) due to the presence of the additional salt ion.

As used herein, “about” in the context of a numerical value or range means ±10% of the numerical value or range recited or claimed.

As used herein, a composition that is “free” of a chemical entity means that the composition contains, if at all, an amount of the chemical entity which cannot be avoided although the chemical entity is not part of the formulation and was not affirmatively added during any part of the manufacturing process.

For example, a composition which is “free” of an alkalizing agent means that the alkalizing agent, if present at all, is a minority component of the composition by weight. Preferably, when a composition is “free” of a component, the composition comprises less than 0.1 wt %, 0.05 wt %, 0.02 wt %, or 0.01 wt % of the component.

As used herein, “alkalizing agent” is used interchangeably with the term “alkaline-reacting component” or “alkaline agent” and refers to any pharmaceutically acceptable excipient which neutralizes protons in, and raises the pH of, the pharmaceutical composition in which it is used.

As used herein, “oxidation reducing agent” refers to a group of chemicals which includes an “antioxidant”, a “reduction agent” and a “chelating agent”.

As used herein, “antioxidant” refers to a compound selected from the group consisting of tocopherol, methionine, glutathione, tocotrienol, dimethyl glycine, betaine, butylated hydroxyanisole, butylated hydroxytoluene, turmerin, vitamin E, ascorbyl palmitate, tocopherol, deteroxime mesylate, methyl paraben, ethyl paraben, butylated hydroxyanisole, butylated hydroxytoluene, propyl gallate, sodium or potassium metabisulfite, sodium or potassium sulfite, alpha tocopherol or derivatives thereof, sodium ascorbate, disodium edentate, BHA (butylated hydroxyanisole), a pharmaceutically acceptable salt or ester of the mentioned compounds, and mixtures thereof.

The term “antioxidant” as used herein also refers to Flavonoids such as those selected from the group of quercetin, morin, naringenin and hesperetin, taxifolin, afzelin, quercitrin, myricitrin, genistein, apigenin and biochanin A, flavone, flavopiridol, isoflavonoids such as the soy isoflavonoid, genistein, catechins such as the tea catechin epigallocatechin gallate, flavonol, epicatechin, hesperetin, chrysin, diosmin, hesperidin, luteolin, and rutin.

As used herein, “reduction agent” refers to a compound selected from the group consisting of thiol-containing compound, thioglycerol, mercaptoethanol, thioglycol, thiodiglycol, cysteine, thioglucose, dithiothreitol (DTT), dithio-bis-maleimidoethane (DTME), 2,6-di-tert-butyl-4-methylphenol (BHT), sodium dithionite, sodium bisulphite, formamidine sodium metabisulphite, and ammonium bisulphite.”

As used herein, “chelating agent” refers to a compound selected from the group consisting of penicillamine, trientine, N,N′-diethyldithiocarbamate (DDC), 2,3,2′-tetraamine (2,3,2′-tet), neocuproine, N,N,N′,N′-tetrakis(2-pyridylmethyl)ethylenediamine (TPEN), 1,10-phenanthroline (PHE), tetraethylenepentamine, triethylenetetraamine and tris(2-carboxyethyl) phosphine (TCEP), ferrioxamine, CP94, EDTA, deferoxainine B (DFO) as the methanesulfonate salt (also known as desferrioxanilne B mesylate (DFOM)), desferal from Novartis (previously Ciba-Giegy), and apoferritin.

As used herein, a pharmaceutical composition is “stable” when the composition preserves the physical stability/integrity and/or chemical stability/integrity of the active pharmaceutical ingredient during storage. Furthermore, “stable pharmaceutical composition” is characterized by its level of degradation products not exceeding 5% at 40° C./75% RH after 6 months or 3% at 55° C./75% RH after two weeks, compared to their level in time zero.

As used herein, “combination” means an assemblage of reagents for use in therapy either by simultaneous or contemporaneous administration. Simultaneous administration refers to administration of an admixture (whether a true mixture, a suspension, an emulsion or other physical combination) of the laquinimod and the GA. In this case, the combination may be the admixture or separate containers of the laquinimod and the GA that are combined just prior to administration. Contemporaneous administration refers to the separate administration of the laquinimod and the GA at the same time, or at times sufficiently close together that a synergistic activity relative to the activity of either the laquinimod or the GA alone is observed.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding laquinimod therapy to a patient already receiving GA therapy.

As used herein, “effective” when referring to an amount of laquinimod and/or glatiramer acetate (GA) refers to the quantity of laquinimod and/or glatiramer acetate (GA) that is sufficient to yield a desired therapeutic response without undue adverse side effects (such as toxicity, irritation, or allergic response) commensurate with a reasonable benefit/risk ratio when used in the manner of this invention.

“Administering to the subject” or “administering to the (human) patient” means the giving of, dispensing of, or application of medicines, drugs, or remedies to a subject/patient to relieve, cure, or reduce the symptoms associated with a condition, e.g., a pathological condition.

“Treating” as used herein encompasses, e.g., inducing inhibition, regression, or stasis of a disease or disorder, e.g., RMS, or lessening, suppressing, inhibiting, reducing the severity of; eliminating or substantially eliminating, or ameliorating a symptom of the disease or disorder. “Treating” as applied to patients presenting CIS can mean delaying the onset of clinically definite multiple sclerosis (CDMS), delaying the progression to CDMS, reducing the risk of conversion to CDMS, or reducing the frequency of relapse in a patient who experienced a first clinical episode consistent with multiple sclerosis and who has a high risk of developing CDMS.

“Inhibition” of disease progression or disease complication in a subject means preventing or reducing the disease progression and/or disease complication in the subject.

A “symptom” associated with RMS includes any clinical or laboratory manifestation associated with RMS and is not limited to what the subject can feel or observe.

As used herein, “a subject afflicted with relapsing multiple sclerosis” means a subject who has been clinically diagnosed to have relapsing multiple sclerosis (RMS) which includes relapsing-remitting multiple sclerosis (RRMS) and Secondary Progressive multiple sclerosis (SPMS).

As used herein, a “responder” to GA treatment refers to a subject that is positively responsive, i.e. the patient's situation improves upon GA therapy. As used herein, a “non-responder” to GA treatment is defined as a subject that does not adequately respond to GA-therapy. A “responder” and a “non-responder” to GA treatment can be measured in any of methods known in the art including that disclosed in PCT International Application No. WO 2006/116602, WO 2012/051106, and U.S. Application Publication No. 2011-0230413, which are hereby incorporated by reference herein.

As used herein, a subject at “baseline” is as subject prior to administration of laquinimod.

A “patient at risk of developing MS” (i.e. clinically definite MS) as used herein is a patient presenting any of the known risk factors for MS. The known risk factors for MS include any one of a clinically isolated syndrome (CIS), a single attack suggestive of MS without a lesion, the presence of a lesion (in any of the CNS, PNS, or myelin sheath) without a clinical attack, environmental factors (geographical location, climate, diet, toxins, sunlight), genetics (variation of genes encoding HLA-DRB1, IL7R.alpha and IL2R-alpha), and immunological components (viral infection such as by Epstein-Barr virus, high avidity CD4⁺ T cells, CD8⁺ T cells, anti-NF-L, anti-CSF 114(Glc)).

“Clinically isolated syndrome (CIS)” as used herein refers to 1) a single clinical attack (used interchangeably herein with “first clinical event” and “first demyelinating event”) suggestive of MS, which, for example, presents as an episode of optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning, and 2) at least one lesion suggestive of MS. In a specific example, CIS diagnosis would be based on a single clinical attack and at least 2 lesions suggestive of MS measuring 6 mm or more in diameter.

“Relapse Rate” is the number of confirmed relapses per unit time. “Annualized relapse rate” is the mean value of the number of confirmed relapses of each patient multiplied by 365 and divided by the number of days that patient is on the study drug.

“Expanded Disability Status Scale” or “EDSS” is a rating system that is frequently used for classifying and standardizing the condition of people with multiple sclerosis. The score ranges from 0.0 representing a normal neurological exam to 10.0 representing death due to MS. The score is based upon neurological testing and examination of functional systems (FS), which are areas of the central nervous system which control bodily functions. The functional systems are: Pyramidal (ability to walk), Cerebellar (coordination), Brain stem (speech and swallowing), Sensory (touch and pain), Bowel and bladder functions, Visual, Mental, and Other (includes any other neurological findings due to MS) (Kurtzke J F, 1983).

A “confirmed progression” of EDSS, or “confirmed disease progression” as measured by EDSS score is defined as a 1 point increase from baseline EDSS sustained for at least 3 months. In addition, confirmation of progression cannot be made during a relapse.

“Adverse event” or “AE” means any untoward medical occurrence in a clinical trial subject administered a medicinal product and which does not have a causal relationship with the treatment.

An adverse event can therefore be any unfavorable and unintended sign including an abnormal laboratory finding, symptom, or diseases temporally associated with the use of an investigational medicinal product, whether or not considered related to the investigational medicinal product.

“Gd-enhancing lesion” refers to lesions that result from a breakdown of the blood-brain barrier, which appear in contrast studies using gandolinium contrast agents. Gandolinium enhancement provides information as to the age of a lesion, as Gd-enhancing lesions typically occur within a six week period of lesion formation.

“Magnetization Transfer Imaging” or “MTI” is based on the magnetization interaction (through dipolar and/or chemical exchange) between bulk water protons and macromolecular protons. By applying an off resonance radio frequency pulse to the macromolecular protons, the saturation of these protons is then transferred to the bulk water protons. The result is a decrease in signal (the net magnetization of visible protons is reduced), depending on the magnitude of MT between tissue macromolecules and bulk water. “MT” or “Magnetization Transfer” refers to the transfer of longitudinal magnetization from the hydrogen nuclei of water that have restricted motion to the hydrogen nuclei of water that moves with many degrees of freedom. With MTI, the presence or absence of macromolecules (e.g. in membranes or brain tissue) can be seen. (Mehta, 1996; Grossman, 1994)

“Magnetization Resonance Spectroscopy” or “MRS” is a specialized technique associated with magnetic resonance imaging (MRI). MRS is used to measure the levels of different metabolites in body tissues. The MR signal produces a spectrum of resonances that correspond to different molecular arrangements of the isotope being “excited”. This signature is used to diagnose certain metabolic disorders, especially those affecting the brain, (Rosen, 2007) as well as to provide information on tumor metabolism. (Golder, 2007)

As used herein “mobility” refers to any ability relating to walking, walking speed, gait, strength of leg muscles, leg function and the ability to move with or without assistance. Mobility can be evaluated by one or more of several tests including but not limited to Ambulation Index, Time 25 foot walk, Six-Minute Walk (6MW), Lower Extremity Manual Muscle Test (LEMMT) and EDSS. Mobility can also be reported by the subject, for example by questionnaires, including but not limited to 12-Item Multiple Sclerosis Walking Scale (MSWS-12). Impaired Mobility refers to any impairment, difficulty or disability relating to mobility.

“T1-weighted MRI image” refers to an MR-image that emphasizes T1 contrast by which lesions may be visualized. Abnormal areas in a T1-weighted MRI image are “hypointense” and appear as dark spots. These spots are generally older lesions.

“T2-weighted MRI image” refers to an MR-image that emphasizes T2 contrast by which lesions may be visualized. T2 lesions represent new inflammatory activity.

The “Six-Minute Walk (6MW) Test” is a commonly used test developed to assess exercise capacity in patients with COPD (Guyatt, 1985). It has been used also to measure mobility in multiple sclerosis patients (Clinical Trials Website).

The “Timed-25 Foot Walk” or “T25-FW” is a quantitative mobility and leg function performance test based on a timed 25-walk. The patient is directed to one end of a clearly marked 25-foot course and is instructed to walk 25 feet as quickly as possible, but safely. The time is calculated from the initiation of the instruction to start and ends when the patient has reached the 25-foot mark. The task is immediately administered again by having the patient walk back the same distance. Patients may use assistive devices when doing this task. The score for the T25-FW is the average of the two completed trials. This score can be used individually or used as part of the MSFC composite score (National MS Society Website).

One of the central symptoms of multiple sclerosis is fatigue. Fatigue can be measured by several tests including but not limited to decrease of French valid versions of the Fatigue Impact Scale (EMIF-SEP) score, and European Quality of Life (EuroQoL) Questionnaire (EQSD). Other tests, including but not limited to Clinician Global Impression of Change (CGIC) and Subject Global Impression (SGI), as well as EQ-5D, can be used to evaluate the general health status and quality of life of MS patients.

“Ambulation Index” or “AI” is a rating scale developed by Hauser et al. to assess mobility by evaluating the time and degree of assistance required to walk 25 feet. Scores range from 0 (asymptomatic and fully active) to 10 (bedridden). The patient is asked to walk a marked 25-foot course as quickly and safely as possible. The examiner records the time and type of assistance (e.g., cane, walker, crutches) needed. (Hauser, 1983)

“EQ-5D” is a standardized questionnaire instrument for use as a measure of health outcome applicable to a range of health conditions and treatments. It provides a simple descriptive profile and a single index value for health status that can be used in the clinical and economic evaluation of health care as well as population health surveys. EQ-5D was developed by the “EuroQoL” Group which comprises a network of international, multilingual, multidisciplinary researchers, originally from seven centers in England, Finland, the Netherlands, Norway and Sweden. The EQ-5D questionnaire is in the public domain and can be obtained from EuroQoL.

“SF-36” is a multi-purpose, short-form health survey with 36 questions which yields an 8-scale profile of functional health and well-being scores as well as psychometrically-based physical and mental health summary measures and a preference-based health utility index. It is a generic measure, as opposed to one that targets a specific age, disease, or treatment group. The survey is developed by and can be obtained from QualityMetric, Inc. of Providence, R.I.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “0.1-2.5 mg/day” includes 0.1 mg/day, 0.2 mg/day, 0.3 mg/day, etc. up to 2.5 mg/day.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Assessment of Add-on Effect of Laquinimod in Mice Treated with Glatiramer Acetate (GA) or Interferon-Beta (IFN-β)

Mice were treated with a sub-optimal dose of Laquinimod (10 mg/kg) alone or add on glatiramer acetate (12.5 mg/kg) or IFN-β (500,000 IU/mouse). In both cases, the combined treatment resulted in improved efficacy when compared to each treatment alone.

### Example 2

### Assessment of Efficacy of Laquinimod in Combination with Glatiramer Acetate (GA) in Rodent EAE Models

Experimental autoimmune encephalomyelitis (EAE) is an animal model (mostly used with rodents) of the human CNS demyelinating diseases, including MS.

### Example 2.1

### MOG Study

This study is designed to test the efficacy of laquinimod in abrogating MOG-induced EAE. Laquinimod was tested alone (5 and 25 mg/kg/day) and in combination with GA blocking.

Materials and Methods:

Induction of EAE:

EAE was induced by subcutaneous injection of encephalitogenic emulsion at a volume of 0.2 ml/mouse in the right flank. On the day of induction, pertussis toxin was injected i.p. at a volume dose of 0.2 ml/mouse. The injection of the pertussis toxin was repeated after 48 hours.

Test Procedure:

Day 0: Subcutaneous injection of MOG into right flank, ip injection of Pertussis toxin. Beginning of daily laquinimod treatment.

Day 2: ip injection of Pertussis toxin.

Day 10: initiation of scoring of mice for EAE clinical signs.

Day 30: termination of study.

Myelin Oligodendrocyte Glycoprotein (MOG) Disease Induction:

On day 0 all mice were injected with the encephalitogenic emulsion (containing 150 μg myelin oligodendrocyte glycoprotein (MOG) and 500 μg M. tuberculosis enriched CFA. Emulsion for groups with GA-blocking included GA (250 μg/mouse). The emulsion was made from equal parts of oil and liquid portions (1:1) in two syringes connected to each other with Leur lock, transferred to insulin syringe and 0.2 ml injected to the right flank of each mouse. On the day of induction and 48 hours thereafter, mice were injected with Pertussis Toxin (ip., 100 ng).

MSCH-Induced EAE in CSJI/F1 Mice:

EAE was induced by injecting the encephalitogenic mixture (emulsion) consisting of MSCH in PBS and commercial CFA containing 1 mg/mL Mycobacterium tuberculosis (in ratio 1:1). A total volume of 50 μl of emulsion was injected in left foot pad of each mouse. Pertussis toxin was injected intravenously on the day of induction and 48 hours later at volume dose of 0.5 ml/mouse).

Laquinimod Treatment in Mice:

The high dose (25 mg/10 ml/kg) solution (150 mg in 60 ml DDW) was prepared once weekly and stored at room temperature in an amber glass vial. For the dose of 5 mg/kg 12 ml of this solution was added to 48 ml DDW. The compound was administered by gavage once daily during the whole experiment (30 days) at a volume of 0.2 ml/mouse. Mice were observed daily from the 10^{th} day post-EAE induction and the EAE clinical signs were scored.

Reagent:

MOG (TV-4915), “Novetide”

Mycobacterium tuberculosis H37 RA (MT)

Pertussis toxin, “Sigma”

Complete freund's adjuvant (CFA), “Sigma”

Study Design:

92 female C57Bl/6 mice 8-10 weeks old were allocated randomly into 6 groups according to Table 1 below.

TABLE 1

| | Dose | | | |
|---|---|---|---|---|
| Group | (mg/kg) | Route | Start | N |
| Control (DDW) | | gavage | | 16 |
| Laquinimod | 5 | gavage | On day 0 | 13 |
| | 25 | gavage | On day 0 | 15 |
| GA-blocking | 12.5 | With inoculum | On day 0 | 16 |
| Laquinimod+ | 5 | gavage | On day 0 | 16 |
| | 25 | gavage | On day 0 | 16 |

GA-blocking was done once, on the day of induction, by inserting GA into the encephalitogenic emulsion. Laquinimod was administered daily throughout the whole experiment

Preparation of Encephalitogenic Emulsion, Pertussis Toxin and Laquinimod:

Oil Portion: CFA (containing 1 mg/ml MT) was enriched to the concentration of 5 mg/ml: 52 mg/MT was added to 13 ml CFA.

Liquid Portion: Groups 1-3: 10.5 mg MOG was diluted in 7 ml PBS (1.5 mg/ml, 150 mg/0.1 ml/mouse). Groups 4-6: 20 mg GA was diluted in 8 ml PBS (2.5 mg/ml, 250 mg/0.1 ml/mouse). 12 mg MOG was added to this solution. The emulsion was made from equal parts of oil and liquid portions (1:1) in two syringes connected to each other with Leur lock, transferred to insulin syringe and 0.2 ml injected to the right flank of each mouse. 50 ml Pertussis toxin (200 mg/ml) was added to 19.95 ml saline to yield 500 ng/ml (100 ng/0.2 ml/mouse). The high dose LAQ (25 mg/10 ml/kg) solution (150 mg in 60 ml DDW) was prepared once weekly and stored at room temperature. For the LAQ dose of 5 mg/kg, 12 ml of this solution was added to 48 ml DDW. The compound was administered by gavage once daily during the whole experiment (30 days) at a volume of 0.2 ml/mouse.

EAE Clinical Signs:

The mice were observed daily from the 10^{th} day post-EAE induction (first injection of MOG) and the EAE clinical signs were scored according to the grades described in the table presented below.

TABLE 2
Evaluation of the EAE clinical signs

| Score | Signs | Description |
|---|---|---|
| 0 | Normal behavior | No neurological signs. |
| 1 | Distal limp tail | The distal part of the tail is limp and droops. |
| 1.5 | Complete limp tail | The whole tail is loose and droops. |
| 2 | righting reflex | Animal has difficulties to return on his feet |
| | | when it is laid on his back |
| 3 | ataxia | wobbly walk -when the mouse walks the |
| | | hind legs are unsteady |
| 4 | early paralysis | The mouse has difficulties standing on its |
| | | hind legs but still has remnants of |
| | | movement. |
| 5 | Full paralysis | The mouse can't move it's legs at all, it |
| | | looks thiner and emaciated. |
| 6 | Moribund/Death | |

Results:

The disease severity in the experiment was much more extreme than expected. There was 56% mortality and group mean score (GMS) reached 3.27.

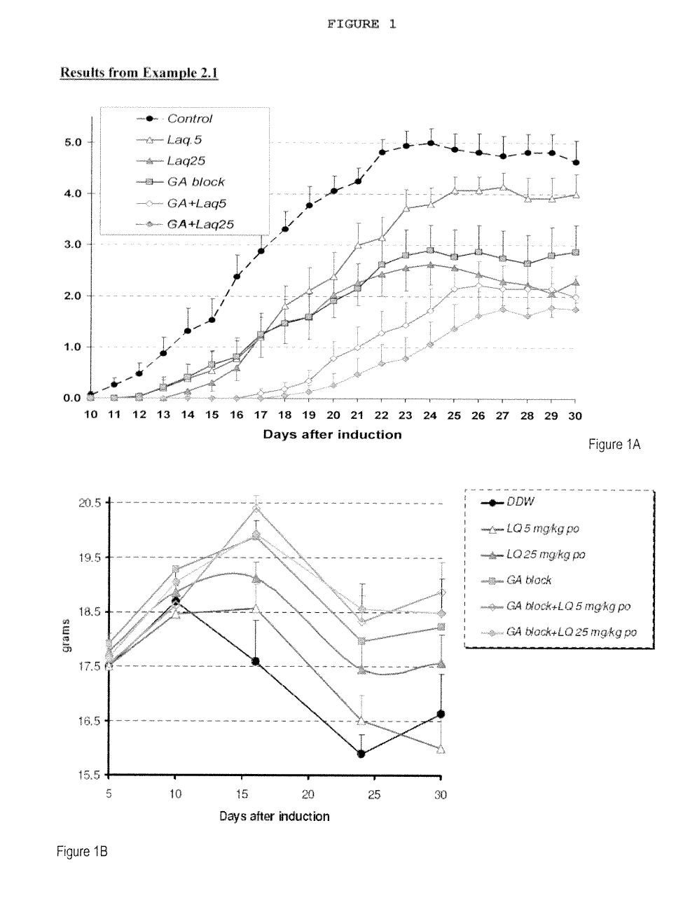

Nevertheless, laquinimod significantly reduced all disease parameters in dose-dependent manner (Table 3, FIG. 1A). Blocking with GA was also effective showing 48% (p=001) inhibition of GMS. Supplemental oral administration of both doses of laquinimod to the same group of mice (blocked by GA) resulted in synergistic effect demonstrating considerable and highly significant decrease in all tested parameters.

The differences in weight gain were correlated with the disease severity—stronger severity resulted in greater weight loss. The differences were statistically significant (by MANOVA). The pair-wise comparisons revealed that during the first ten days after induction all the groups gained weight quite equally. However, when the first signs appeared the mice started to lose weight. On days 16 and 24 the weight of control mice was statistically different from almost all the others (Table 4, FIG. 1B).

TABLE 3

| | | | Parameter | | | | | | | |
|---|---|---|---|---|---|---|---|---|---|---|
| | | | | | | | % | | | Mean |
| Group | | | | | Mean | Group | inhibition | | Mean | Disease |
| | | dose, | | Mortal- | Maximal | Mean | compared | compared | Onset, | Duration, |
| | | mg/kg | Incidence | ity | Score | Score | to control | to GA-block | days | days |
| Control | | — | 16/16 | 9/16 | 5.25 ± 0.9 | 3.27 ± 0.8 | | | 14.6 ± 3.0 | 16.4 ± 3.0 |
| laquinimod | | 5 | 13/13 | 2/13 | 4.38* ± 0.8 | 2.25*** ± 0.7 | 31.2 | | 16.8* ± 2.8 | 13.2** ± 3.1 |
| | | 25 | 14/15 | 0/15 | 3.27*** ± 1.3 | 1.49*** ± 0.9 | 54.4 | | 20.3** ± 5.9 | 10.0*** ± 5.5 |
| GA blocking | | 250 | 14/16 | 2/16 | 3.34** ± 1.7 | 1.70** ± 1.3 | 48.0 | | 19.5* ± 5.8 | 10.9** ± 5.8 |
| GA | laq. | 5 | 12/16 | 0/16 | 2.50*** ± 1.8 | 0.94*** ± 0.8 | 71.3 | 44.7 | 24.1*** ± 5.0 | 6.4*** ± 5.2 |
| blocking + | | 25 | 9/16 | 0/16 | 2.0*** ± 1.9 | 0.64*** ± 0.7 | 80.4 | 62.4* | 26.4*** ± 4.8 | 4.6*** ± 4.8 |

*p < 0.05;
**p < 0.005;
***p < 0.0005

TABLE 4

| | | dose, | Days after induction | | | | |
|---|---|---|---|---|---|---|---|
| Group | | mg/kg | 5 | 10 | 16 | 24 | 30 |
| Control | | — | 17.5 ± 0.16 | 18.7 ± 0.30 | 17.6 ± 0.75 | 15.9 ± 0.36 | 16.6 ± 0.74 |
| laquinimod | | 5 | 17.5 ± 0.27 | 18.5 ± 0.28 | 18.6 ± 0.44 | 16.5 ± 0.45 | 16.0 ± 0.63 |
| | | 25 | 17.8 ± 0.33 | 18.9 ± 0.36 | 19.1* ± 0.3 | 17.5 ± 0.52 | 17.6 ± 0.5 |
| GA blocking | | 250 | 17.9 ± 0.16 | 19.3 ± 0.25 | 19.9** ± 0.63 | 18.0* ± 0.63 | 18.2 ± 0.71 |
| GA | laq. | 5 | 17.5 ± 0.17 | 18.6 ± 0.21 | 20.4*** ± 0.22 | 18.3** ± 0.49 | 18.9* ± 0.53 |
| blocking + | | 25 | 17.6 ± 0.17 | 19.1 ± 0.21 | 19.9** ± 0.24 | 18.5** ± 0.48 | 18.5 ± 0.64 |

*p < 0.05;
**p < 0.005;
***p < 0.0005

Conclusion:

Laquinimod given alone significantly reduced all disease parameters in a dose-dependent manner. Combination of oral laquinimod with GA-blocking resulted in an additive effect where the inhibition of disease score was greater than with each compound alone.

Although the disease was quite severe, laquinimod in both doses delayed the onset of the first signs and ameliorated the pathological symptoms. Given in combination with GA-blocking, laquinimod was extremely effective demonstrating up to 80% inhibition of GMS.

### Example 2.2

### MOG Study

This study was designed to test lower doses of laquinimod alone and in combination with GA-blocking. MOG is a member of the immunoglobulin superfamily expressed exclusively in CNS myelin. It is one of the most encephalitogenic proteins and is widely used to induce EAE in different rodent strains. In C57BL/6 mice, immunization with the MOG peptide pMOG35-55 in CFA induces chronic progressive EAE. In this study, the descending doses of laquinimod were tested in GA-blocked mice.

Materials and Methods:

EAE induction and Test Procedure are same as in Example 2.1. Reagents are same as in Example 2.1

Study Design:

105 female C57Bl/6 mice 8-10 weeks old were divided into 7 groups according to Table 5 below.

TABLE 5

| | Dose | | | |
|---|---|---|---|---|
| Group | (mg/kg) | Route | Start | N |
| Control (DDW) | | gavage | | 15 |
| GA-blocking | 12.5 | | On day 0 | 15 |
| Laquinimod | 5 | gavage | On day 0 | 15 |
| Laquinimod + GA-blocking 12.5 | 5 | gavage | On day 0 | 15 |
| Laquinimod | 2.5 | gavage | On day 0 | 15 |
| Laquinimod + GA-blocking 12.5 | 2.5 | gavage | On day 0 | 15 |
| Laquinimod + GA-blocking 12.5 | 1.0 | gavage | On day 0 | 15 |

Preparation of Encephalitogenic Emulsion, Pertussis Toxin and Laquinimod:

Oil Portion:

CFA (containing 1 mg/ml MT) was enriched to the concentration of 4 mg/ml: 45 mg MT was added to 15 ml CFA.

Liquid Portion:

24 mg MOG was diluted in 8 mil PBS (3 mg/ml, stock solution). Groups 1, 3 and 5: 3.5 ml of stock solution was diluted 1:1 with PBS. Groups 2, 4, 6, and 7: 22.5 mg GA was diluted in 4.5 ml PBS (5 mg/ml). 4.5 ml of MOG stock solution was added to this solution.

The emulsion was made from equal parts of oil and liquid portions (1:1) in two syringes connected to each other with Leur lock, transferred to insulin syringe and 0.2 ml was injected to the right flank of each mouse.

41 μl Pertussis toxin (200 μg/ml) was added to 21.96 ml saline to yield 375 ng/ml (75 ng/0.2 ml/mouse).

The high dose LAQ (5 mg/10 ml/kg) solution (40 mg in 80 ml DDW) was prepared once weekly and stored at room temperature. For the dose of 2.5 mg/kg LAQ, 25 ml of this solution were added to 25 mil DDW. For the dose 1 mg/kg 5 ml of stock solution (high dose) were added to 20 ml DDW. The compound was administered by gavage once daily during the whole experiment (30 days) at a volume of 0.2 ml/mouse.

EAE Clinical Signs:

The mice were observed daily from the 9^{th} day post-EAE induction (first injection of MOG) and the EAE clinical signs were scored according to the grades described in the 2 above.

Results:

In this study, the control group reached a high GMS of 3.24 despite the fact that the concentration of mycobacterium tuberculosis in CFA was decreased from 5 to 4 mg/ml. Nevertheless, all the treatments significantly reduced this value (Table 6).

TABLE 6
The effect of laquinimod alone and in combination with GA-blocking on disease severity.

| | | | Parameter | | | | | | | |
|---|---|---|---|---|---|---|---|---|---|---|
| | | | | | | | % | | | Mean |
| Group | | | | | Mean | Group | inhibition | | Mean | Disease |
| | | dose, | Inci- | Mortal- | Maximal | Mean | compared | compared | Onset, | Duration, |
| | | mg/kg | dence | ity | Score | Score | to control | to GA-block | days | days |
| Control | | — | 15/15 | 3/15 | 4.60 ± 0.9 | 3.24 ± 1.0 | | | 10.8 ± 1.7 | 19.2 ± 3.3 |
| laquinimod | | 2.5 | 13/15 | 1/15 | 3.17** ± 1.6 | 1.50** ± 1.1 | 53.7 | | 18.9*** ± 6.6 | 10.2*** ± 5.6 |
| | | 5 | 12/14 | 0/14 | 2.89** ± 1.6 | 1.03*** ± 0.9 | 68.2 | | 18.1*** ± 6.5 | 8 6*** ± 6.0 |
| GA blocking | | 12.5 | 11/14 | 0/14 | 2.18*** ± 1.6 | 0.73*** ± 0.7 | 77.5 | | 18.7*** ± 6.8 | 7.1*** ± 5.1 |
| GA | laq | 1 | 14/15 | 0/15 | 3.73* ± 1.0*** | 1.71*** ± 0.7 | 47.2 | −134.2 | 19.5*** ± 4.3*** | 11.1*** ± 4.1 |
| blocking | | 2.5 | 10/14 | 0/14 | 2.71** ± 1.9*** | 1.27*** ± 1.1 | 60.8 | −74.0 | 22.1*** ± 6.9*** | 8 5*** ± 6.5 |
| (12.5) + | | 5 | 7/15 | 0/15 | 1.87** ± 2.1 | 0.70*** ± 0.9 | 78.4 | 4.1 | 25.8*** ± 6.5 | 4.8*** ± 5.9 |

*p < 0.05;
**p < 0.005;
***p < 0.0005

GA-blocking was very effective in inhibition of all tested parameters. Laquinimod given alone showed dose-dependent efficacy (up to 68% improvement of GMS).

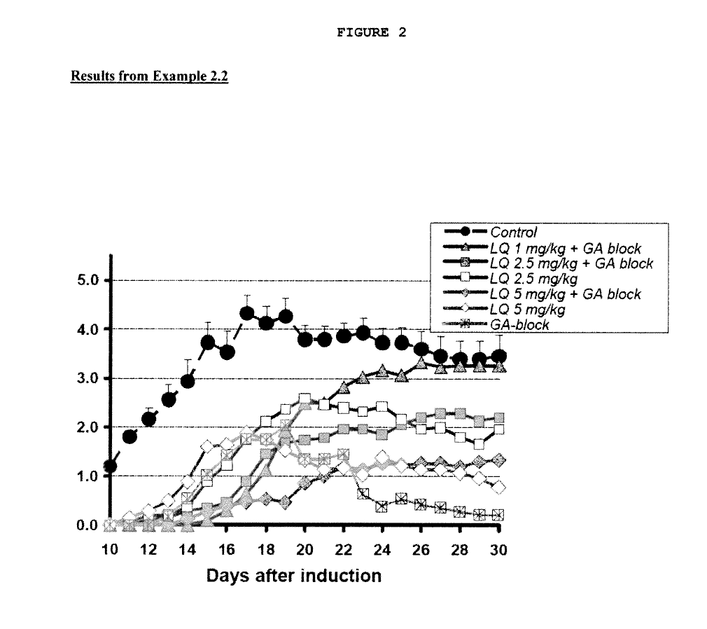

The combination of both compounds did not show any synergistic effect, as shown in previous studies. In the present study a supplement of oral laquinimod to GA-blocked mice seems to improve the neurological signs at the beginning of the disease (FIG. 2).

Evidently, the onset of the disease is delayed by one to six days dose-dependently. However, from days 18-19 the GA-blocked mice started to recover but the mice with combined treatment continued to develop signs of progressive disease. After the final calculation, it looks like the combined treatment of GA-blocking and oral laquinimod results in a negative interaction (in descending dose-response manner).

Laquinimod given alone showed dose-dependent efficacy (up to 68% improvement of Group Mean Score (GMS)). The combination of both compounds did not show any synergistic effect, which may be due to the fact that GA-Blocking resulted in 77.5% inhibition which made it difficult to see any additive effect of combination studies. In the present study addition of oral laquinimod to GA-blocked mice seemed to improve the neurological signs at the beginning of the disease. Evidently, the onset of the disease is delayed by one to six days dose-dependently.

Conclusions:

In the previous studies it was found that daily oral laquinimod given to mice with GA-blocked EAE significantly improved disease manifestation as compared to GA-blocking only. In these studies relatively high doses (5 and 25 mg/kg) of laquinimod were tested.

The present study was the first to test the smaller doses.

In conclusion, the general impression is that a high dose of laquinimod (25 mg/kg) in combination with GA-blocking has marked additive effect (but is unfavorable due to toxicological findings), the intermediate dose (5 mg/kg) once was effective (45% improvement as compared to GA-blocking) and this time had comparable results. The small doses (2.5 and 1 mg/kg) were tested only in the current study and apparently showed the negative interaction with GA-blocking.

### Example 2.3

### MOG Study—Repeat Study

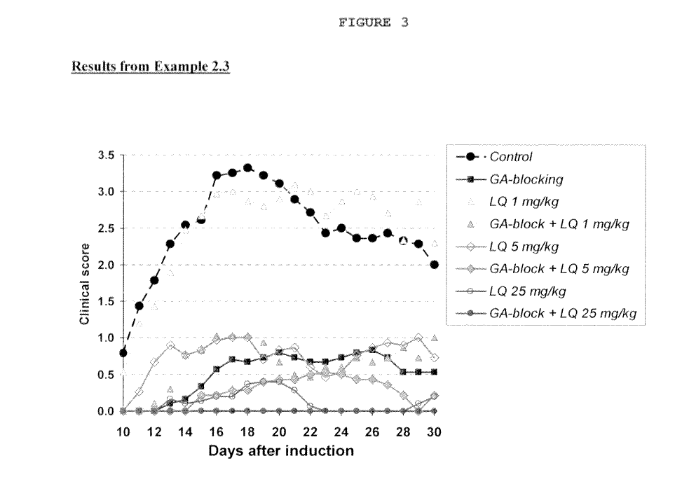

In examples 2.1 and 2.2 the higher dose laquinimod had a clear synergistic effect while lower doses apparently did not provide any additive effect over GA-blocking. Example 2.2 is repeated here. Results are shown on Table 6 and FIG. 3. Disease induction and treatment were carried out as in example 2.1.

Materials and Methods:

EAE induction and Test Procedure are same as in Example 2.1. Reagents are same as in Example 2.1

Study Design:

120 female C57Bl/6 mice 8-10 weeks old were used the study. Mice were allocated randomly into 8 groups according to Table 7 below.

TABLE 7

| Group | Dose | Route | Start | N |
|---|---|---|---|---|
| Control (DDW) | | gavage | On day 0 | 15 |
| GA-blocking | 12.5 | With inoculum | On day 0 | 15 |
| Laquinimod | 1 | gavage | On day 0 | 15 |
| Laquinimod + GA-blocking 12.5 | 1 | gavage | On day 0 | 15 |
| Laquinimod | 5 | gavage | On day 0 | 15 |
| Laquinimod + GA-blocking 12.5 | 5 | gavage | On day 0 | 15 |
| Laquinimod | 25 | gavage | On day 0 | 15 |
| Laquinimod + GA-blocking 12.5 | 25 | gavage | On day 0 | 15 |

Preparation of Encephalitogenic Emulsion

Oil Portion:

CFA (containing 1 mg/ml MT) was enriched to the concentration of 2 mg/m: 16 mg/MT was added to 16 ml CFA.

Liquid Portion:

24 mg MOG was diluted in 8 ml PBS (3 mg/ml, stock solution). Groups 1, 3, 5 and 7: 4 ml of stock solution was diluted 1:1 with PBS. Groups 2, 4, 6, and 8: 22.5 mg GA was diluted in 4.5 ml PBS (5 mg/ml). This solution was mixed 1:1 with the stock solution of MOG.

The emulsion was made from equal parts of oil and liquid portions (1:1) in two syringes connected to each other with Leur lock, transferred to insulin syringe and 0.2 ml was injected to the right flank of each mouse.

Preparation of Pertussis Toxin

31.875 μl Pertussis toxin (200 μg/ml) was added to 25.468 ml saline to yield 250 ng/ml (50 ng/0.2 ml/mouse).

Preparation of Laquinimod

The high dose LAQ (25 mg/10 ml/kg) solution (150 mg in 60 ml DDW) was prepared once weekly and stored in the amber glass at room temperature. The lower LAQ doses were prepared daily. For the dose of 5 mg/kg, 1.6 ml of stock solution were added to 6.4 ml DDW. For the dose 1 mg/kg, 1.3 ml of the previous solution (intermediate dose) were added to 5.2 ml DDW. The compound was administered by gavage once daily during the hole experiment (30 days) at a volume of 0.2 ml/mouse.

EAE Clinical Signs

The mice were observed daily from the 10^{th} day post-EAE induction (first injection of MOG) and the EAE clinical signs were scored according to the grades described in the 2 above.

Results

As in the previous study, GA-blocking was effective in inhibition of all tested parameters of the disease (Table 8).

In general, laquinimod alone showed dose-depended efficacy from no effect at a dose of 1 mg/kg to 70% GMS reduction at 5 mg/kg and 95% inhibition of GMS at 25 mg/kg.

Given in combination with GA-blocking, laquinimod at low dose did not show additive effect, but two higher doses surpassed the effect of GA either reducing GMS of control group by −90% (5 mg/kg) or completely inhibiting the disease (no mouse was sick, 25 mg/kg).

TABLE 8
The effect of laquinimod alone and in combination with GA-blocking on disease severity

| | | | Parameter | | | | | | | |
|---|---|---|---|---|---|---|---|---|---|---|
| | | | | | | | | | | Mean |
| Group | | | | | Mean | Group | % inhibition | | Mean | Disease |
| | | dose, | | | Maximal | Mean | copmared to | | Onset, | Duration, |
| | | mg/kg | Incidence | Mortality | Score | Score | Control | GA-block | days | days |
| Control | | — | 14/14 | 4/14 | 3.93 ± 1.6 | 2.47 ± 1.9 | | | 12.1 ± 3.0 | 14.6 ± 5.6 |
| laquinimod | | 1 | 15/15 | 4/15 | 4.27 ± 1.2 | 2.50 ± 1.8 | −1.2 | | 13.7 ± 4.0 | 14.1 ± 5.3 |
| | | 5 | 11/15 | 0/15 | 2.23* ± 1.5 | 0.74** ± 0.7 | 70.0 | | 21.0*** ± 8.4 | 6.3*** ± 5.0 |
| | | 25 | 6/15 | 0/15 | 0.77*** ± 1.1 | 0.12*** ± 0.2 | 95.1 | | 24.7*** ± 8.1 | 1.5*** ± 2.3 |
| GA blocking | | 13 | 5/15 | 0/15 | 0.97*** ± 1.6 | 0.52*** ± 1.0 | 78.9 | | 25.6*** ± 7.9 | 3.7*** ± 6.4 |
| GA | laq | 1 | 9/15 | 0/15 | 1.80** ± 1.7 | 0.65** ± 0.9 | 73.7 | −25.0 | 23.3*** ± 7.9 | 5.1*** ± 5.8 |
| blocking | | 5 | 3/14 | 0/14 | 0.61*** ± 1.3 | 0.26*** ± 0.6 | 89.5 | 50.0 | 28.4*** ± 5.4 | 1.9*** ± 4.4 |
| (12.5) + | | 25 | 0/15 | 0/15 | 0*** | 0*** | 100.0 | 100* | 31*** | 0*** |

*p < 0.05;
**p < 0.005;
***p < 0.0005

The statistical analysis was made using Kruskal-Wallis tat for non-parametric variables followed by Mann-Whitney comparison

Laquinimod alone showed dose-depended efficacy from no effect at a dose of 1 mg/kg to 70% a GMS reduction at 5 mg/kg and 95% inhibition of GMS at 25 mg/kg.

Despite the large effect seen with GA-blocking alone (78.9%), laquinimod given in combination showed additive effect at doses of 5 mg/kg (89.5%) and 25 mg/kg, where the higher dose resulted in complete inhibition of disease.

Since GA-blocking was markedly effective in this study, it was difficult to obtain a more significant improvement of this result by combined treatment with oral laquinimod. In order to achieve better combination effect, the dose of GA is reduced to suboptimal.

Summary of Studies of GA-Blocking+Laquinimod in MOG-Induced EAE (Examples 2.1-2.3

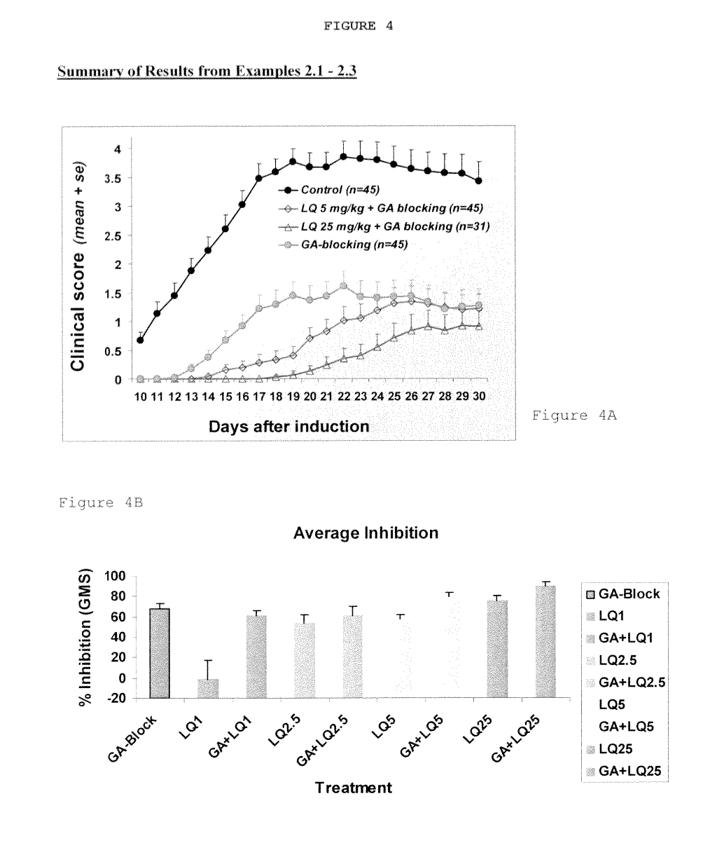

At higher doses (5 and 25 mg/kg) laquinimod provides an additive effect over GA-blocking as summarized in Table 9 and shown on FIGS. 4A and 4B.

TABLE 9

| | | | | | GA- | laquinimod + | | | |
|---|---|---|---|---|---|---|---|---|---|
| | laquinimod | | | | Block- | GA Blocking | | | |
| | 1 | 2.5 | 5 | 25 | ing− | 1 | 2.5 | 5 | 25 |
| Mean | 1.3− | 53.6 | 57.3 | 74.88 | 67.6 | 60.4 | 60.87 | 79.4 | 90.0 |
| n | 15 | 15 | 42 | 30 | 45 | 30 | 14 | 45 | 31 |
| SD | 72.1 | 32.4 | 30.6 | 29.0 | 37.7 | 32.5 | 33.0 | 27.2 | 18.5 |
| se | 18.6 | 8.4 | 4.7 | 5.3 | 5.6 | 5.9 | 8.8 | 4.0 | 3.3 |

### Example 2.4

### MOG-Induced EAE with GA Blocking and Subcutaneous (s.c). Daily GA

The objective of this study was to test the additive effect of laquinimod and GA in the EAE model in C57Bl mice. The C57Bl strain of mouse was selected, as it is an established EAE model.

Laquinimod was administered daily orally for 30 days at dose level of 5.0 mg/kg or 25.0 mg/kg. GA was administered subcutaneously for 10 days at a dose of 250 mg/kg or once, along with encephalitogen at dose level of 12.5 mg/kg.

In order to study the additive effect of laquinimod and GA, laquinimod was administered to groups of mice where GA was administered subcutaneously at a dose of 250 mg/kg or along with encephalitogen at dose level of 12.5 mg/kg.

The suppressive activity of groups with combination treatment was compared to group where laquinimod or GA were administered alone.

GA at a dose of 12.5 mg/kg administered along with the encephalitogen (blocking) served as positive control group.

General Design

Disease was induced in the mice by the injection of the encephalitogenic emulsion (MOG/CFA). Laquinimod was administered orally for 30 consecutive days from the initiation of the study. GA was administered subcutaneously for 10 consecutive days from the initiation of the study.

Materials

Laquinimod sodium; Glatiramer acetate; Purified water; Pertussis toxin: Sigma; MOG 35-55: Mnf Novatide; Complete Freund's Adjuvant (CFA): Sigma; Saline: Mnf− DEMO S.A; and Mycobacterium tuberculosis H37 RA (MT): Mnf− Difco.

### Experimental Animals

### Species, Strain and Supplier

Healthy, nulliparous, non-pregnant female mice of the C57Bl strain weighing about 15-22 g were approximately 7-8 weeks of age on arrival. The body weight of the animals was recorded on the day of delivery.

Overtly healthy animals were assigned to study groups arbitrarily before treatment commenced.

Test Procedures

EAE induction EAE was induced by injecting the encephalitogenic mixture (emulsion) consisting of MOG (150.0 μg/mouse). A volume of 0.2 ml of emulsion was injected subcutaneously into the flanks of the mice. Pertussis toxin was injected intraperitoneally on the day of induction and 48 hours later (total amount was 0.300 μg/mouse in 0.2 ml dosage volume).

Group Assignment

The mice were allocated to the following treatment groups as shown in Table 10:

TABLE 10

| | Treatment | Administration |
|---|---|---|
| Group | Groups | schedule |
| 1 | Negative Control - purified water (oral) | Daily for 30 days |
| 2 | GA blocking - 12.5 mg/kg (subcutaneous | Once |
| | along with encephalitogen) | |
| 3 | GA blocking - 12.5 mg/kg (subcutaneous | GA - once + |
| | along with encephalitogen) + | laquinimod - daily |
| | laquinimod - 25.0 mg/kg (oral) | for 30 days |
| 4 | GA - 250 mg/kg (subcutaneous) | Daily for 10 days |
| 5 | Laquinimod - 5.0 mg/kg (oral) | Daily for 30 days |
| 6 | Laquinimod - 25.0 mg/kg (oral) | Daily for 30 days |
| 7 | GA - 250 mg/kg (subcutaneous) + | GA - daily for 10 |
| | laquinimod - 5.0 mg/kg (oral) | days + laquinimod - |
| | | daily for 30 days |
| 8 | GA - 250 mg/kg (subcutaneous) + | GA - daily for 10 |
| | laquinimod - 25.0 mg/kg (oral) | days + laquinimod - |
| | | daily for 30 days |

Preparation and Administration of Encephaltogenic Emulsion

Oil Portion:

CFA (containing 1 mg/ml MT) was enriched to the concentration of 2 mg/ml: 30.0 mg/MT was added to 30.0 ml CFA.

Liquid Portion:

Groups 1, 4-8: 46.3 mg MOG was diluted in 15.4 ml Normal Saline (3.0 mg/ml MOG stock solution). 11.0 mL of 3.0 mg/m stock solution solution was diluted in 11.0 mL Normal Saline to yield 1.5 mg MOG/mL. Groups 2 and 3: 5 mg/mL stock solution of GA was prepared in Normal Saline. 4.0 mL of this solution was mixed with 4.0 mL of 3 mg/ml MOG stock solution to yield 1.5 mg MOG/mL and 2.5 mg GA/mL.

The emulsion (for groups 2 and 3 and groups 1 and 4 to 8) were made from equal parts of oil and liquid portions (1:1) in two syringes connected to each other with Leur lock, transferred to insulin syringe and 0.2 ml was injected to the right flank of each mouse. The dose of the MOG in all the groups (1 to 8) was 150 μg/mouse. The dose of the GA in groups 2 and 3 was 250 μg/mouse.

Preparation and Administration of Pertussis Toxin

75 μL Pertussis toxin (200 μg/ml) was added to 40 ml saline to yield about 375 ng/ml. The pertussis toxin was administered intraperitoneally on the day of encephalitogen injection and 48 hours later (75.0 ng/0.2 ml/mouse).

Preparation and Administration of the Test Formulations Laquinimod:

The test concentrations of laquinimod were prepared in purified water. For high dose (25.0 mg/kg) 2.5 mg/mL stock solution were prepared (groups #3, 6 and 8). The stock solutions were diluted 1:5 to yield 0.5 mg/mL for dose level of 5.0 mg/kg for groups 5 and 7. Laquinimod was administered to the respective groups by gavage at a volume of 0.2 ml/mouse.

Glatiramer Acetate:

A 50.0 mg/ml stock solution of GA was prepared in Saline. 8.0 mL of 50.0 mg/mL GA was aliquoted in 10 tubes and stored at −20° C. Every day one tube was thawed and brought to RT. GA was administered subcutaneously daily for 10 days from the initiation of the study to the mice of groups 4, 7 and 8 at 0.1 mL.

### Experimental Observations

### Morbidity and Mortality

All animals were examined once daily to detect if any is dead or moribund.

Clinical Signs

Scoring of EAE clinical signs was started from Day 10 post EAE induction and was continued daily until Day 30. The clinical signs were recorded according to a grading system described in the table 11 below

TABLE 11
Evaluation of the EAE Clinical Signs.

| Score | Signs | Description |
|---|---|---|
| 0 | Normal behaviour | No neurological signs. |
| 1 | Tail weakness | The mouse tail was limp and droops. |
| 2 | Hind legs weakness | Limb paresis, wobbly walk - when the |
| | | mouse walks the hind legs were unsteady. |
| 3 | Hind legs paralysis | The mouse can't move its hind legs and it |
| | | drags them when he walks. |
| 4 | Full paralysis | The mouse can't move it's legs at all. it |
| | | looks thinner and emaciated. |
| 5 | Death | — |

mice having scores of 1 and above were considered sick. When the first clinical sign appeared all mice were given food soaked in water, which was spread on different places on the bedding of the cages. An animal which continued to have score 4 for three days was killed on humane grounds and given score 5 on the next day. For calculation purposes, the score of animals that were sacrificed or died was carried forward.

Interpretation of Results

Calculation of the Incidence of Disease (Disease Ratio)
The number of sick animals in each group were summed.
The incidence of disease was calculated as

$$INCIDENCE of DISEASE = ( \frac{No . of sick mice in treated group}{No . of sick mice in control group} )$$

The percent inhibition according to incidence was calculated as

$$INHIBITION ( \% ) of INCIDENCE = ( 1 - \frac{Number of sick mice in treated group}{Number of sick mice in control group} ) \times 100$$

Calculation of the Mortality/Morbidity Rate (Mortality Ratio)
The number of dead or moribund animals in each group were summed.
The mortality of disease was calculated as

$$MORTALIY o f DISEASE = ( \frac{No . of dead or moribound mice in treated group}{No . of dead or moribound mice in control group} )$$

The percent inhibition according to mortality was calculated as

$$INHIBITION ( \% ) of MORTALITY = ( 1 - \frac{Number of dead or moribound mice in treated group}{Number of dead or moribound mice in control group} ) \times 100$$

Calculation of Duration of Disease
The mean duration of disease expressed in days was calculated as

$$Mean Duration = ( \frac{\sum Duration of disease of each mouse}{No . of mice in the group} )$$

Calculation of Mean Delay in Onset of Disease
The mean onset of disease expressed in days was calculated as

$$Mean Onset = ( \frac{\sum Onset of disease of each mouse}{No . of mice in the group} )$$

The mean delay in onset of disease expressed in days was calculated by subtracting the mean onset of disease in control group from test group.

Calculation of the Mean Maximal Score and Percent Inhibition
The mean maximal score (MMS) of each group was calculated as

$$MMS = ( \frac{\sum Maximal Score of each mouse}{No . of mice in the group} )$$

The percent inhibition according to MMS was calculated as

$$INHIBITION ( \% ) of MMS = ( 1 - \frac{MMS of treated group}{MMS of control group} ) \times 100$$

Calculation of the Group Mean Score and Percent Inhibition
The daily scores of each mouse in the test group were summed and the individual mean daily score (IMS) was calculated as

$$IMS = ( \frac{\sum Daily score of mouse}{Observation period ( days )} )$$

The mean group score (GMS) was calculated as

$$GMS = ( \frac{\sum IMS of each mouse}{No . of mice in the group} )$$

The percent inhibition was calculated as

$$INHIBITION ( \% ) of GMS = ( 1 - \frac{GMS of treated group}{GMS of control group} ) \times 100$$

Results

A summary of the incidence, mortality, MMS, GMS, duration of the disease, onset of the disease and the activity of each group according to Incidence, MMS and GMS is shown in the summarized Table 12.

Clinical Signs and Mortality

No clinical symptoms were observed during the first 10 days of the study (before starting of scoring for EAE). There was no morbidity and mortality prior to onset of the EAE disease.

Incidence, Onset and Duration of Disease

The incidence of disease in the vehicle treated negative control group was 17/20 compared to 8/20 in the GA blocking positive control group. No mortality was observed in the other treated groups.

Mean Maximal Scare (MMS) and Group Mean Score (GMS)

The MMS of the vehicle treated negative control group was 3.4 and of GA blocking positive control group was 0.5.

Group treated with laquinimod (5 mg/kg) along with GA at dose level of 250 mg/kg (subcutaneous) exhibited additive effect (85.2% according to GMS) which was not significant because of high activity exhibited by laquinimod alone (71.4% according to GMS). GA administered subcutaneously exhibited 38.1% activity.

According to GMS, the GA blocking positive control and laquinimod (25 mg/kg) treated groups suppressed EAE compared to the negative control group by 76.2% (p=0.00001) and 95.2% (p>0.00001) respectively. In group where the combination treatment (GA blocking and laquinimod—25 mg/kg) was given, 100% activity was observed.

Conclusion

The test was valid as both calculated parameters of the negative control group, the incidence (85%) and Mean Maximal Score (3.4) met with the acceptance criteria. In the present study, the group treated with laquinimod (5 mg/kg) along with GA at dose level of 250 mg/kg (subcutaneous) exhibited additive effect (85.2%). However the additive effect was not significant because of high activity exhibited by laquinimod alone (71.4%).

TABLE 12

| | | | | | | | | Mean |
|---|---|---|---|---|---|---|---|---|
| | | | | | | | | Duration |
| | | | | MMS | | GMS | | (days) |
| | | | % | MMS | % | GMS | % | Onset |
| Treatment | Mortality | Incidence | Inhibition 1 | value | Inhibition 2 | value | Inhibition 3 | (days) |
| Negative Control - | 1/20 | 17/20 | — | 3.4 ± 1.6 | — | 2.1 ± 1.2 | — | 12.1 ± 6.5 |
| purified water (oral) | | | | | | | | 19.0 ± 6.5 |
| Positive Control - | 0/20 | 8/20 | 52.9% | 1.0 ± 1.5 | 70.6% | 0.5 ± 0.9 | 76.2% | 4.3 ± 6.3 |
| GA blocking - 12.5 | | | | | | | (p = 0.00001) | 26.6 ± 6.4 |
| mg/kg | | | | | | | | |
| GA blocking - | 0/20 | 0/20 | 100.0% | 0.0 ± 0.0 | 100% | 0.0 ± 0.0 | 100% | 0.0 ± 0.0 |
| 12.5 mg/kg + LAQ - | | | | | | | (p > 0.00001) | 31.0 ± 0.0 |
| 25.0 mg/kg (oral) | | | | | | | | |
| GA - 250 mg/kg | 0/20 | 13/20 | 23.5% | 2.5 ± 1.9 | 26.5% | 1.3 ± 1.1 | 38.1% | 8.3 ± 6.7 |
| (subcutaneous) | | | | | | | (p = 0.7) | 22.5 ± 6.7 |
| LAQ 5.0 | 0/20 | 8/10 | 52.9% | 1.5 ± 1.8 | 55.9% | 0.6 ± 0.9 | 71.4% | 4.2 ± 6.0 |
| mg/kg (oral) | | | | | | | (p = 0.00006) | 26.9 ± 6.0 |
| LAQ 25.0 mg/kg | 0/20 | 2/20 | 88.2% | 0.3 ± 1.0 | 91.2% | 0.1 ± 0.4 | 95.2% | 0.8 ± 2.6 |
| (oral) | | | | | | | (p > 0.00001) | 30.3 ± 2.6 |
| GA - 250 mg/kg | 0/20 | 7/20 | 58.8% | 1.1 ± 1.6 | 67.6% | 0.3 ± 0.6 | 85.7% | 2.3 ± 3.8 |
| (subcutaneous) + | | | | | | | (p > 0.00001) | 28.8 ± 3.8 |
| LAQ - 5.0 mg/kg | | | | | | | | |
| (oral) | | | | | | | | |
| GA - 250 mg/kg | 0/20 | 2/20 | 88.2% | 0.4 ± 1.1 | 88.2% | 0.1 ± 0.2 | 95.2% | 0.5 ± 1.6 |
| (subcutaneous) + | | | | | | | (p > 0.00001) | 30.5 ± 1.6 |
| LAQ - 25.0 mg/kg | | | | | | | | |
| (oral) | | | | | | | | |

### Example 2.5

### MOG-Induced EAE with GA Blocking and Subcutaneous (s.c). Daily GA

The objective of this study was to test the suppressive effect of laquinimod alone and in combination with Glatiramer acetate in the EAE model in C57Bl mice. In order to study the additive effect, laquinimod and GA were administered alone or in combination and the suppressive activity of the groups with combination treatment were compared to group where laquinimod or GA were administered alone. GA was administered subcutaneously for 10 days at a dose of 250 mg/kg. Laquinimod was administered orally at dose levels of 2.5 and 5.0 mg/kg. GA at a dose of 12.5 mg/kg administered along with the encephalitogen (blocking) served as positive control group.

The EAE is an animal model for multiple sclerosis. The C57Bl strain of mouse has been selected, as it is an established EAE model

General Design

EAE was induced in the mice by the injection of the encephalitogenic emulsion (MOG/CFA). Laquinimod was administered orally for 30 consecutive days. GA was administered subcutaneously for 10 consecutive days from the initiation of the study.

Materials

Laquinimod sodium; Glatiramer acetate; Purified water, Pertussis toxin: Sigma; MOG 35-55: Mnf Novatide; Complete Freund's Adjuvant (CFA): Sigma; Saline: Mnf-DEMO S.A; and Mycobacterium tuberculosis H37 RA (MT): Mnf-Difco.

### Experimental Animals

Healthy, nulliparous, non-pregnant female mice of the C57Bl strain weighing about 15-22 g were approximately 7-8 weeks old on arrival. The body weights of the animals were recorded on the day of delivery. Overtly healthy animals were assigned to study groups arbitrarily before treatment commenced.

Test Procedures

EAE Induction

EAE was induced by injecting the encephalitogenic mixture (emulsion) consisting of MOG (150.0 mg/mouse). A 0.2 ml emulsion was injected subcutaneously into the flanks of the mice. Pertussis toxin was injected intraperitoneally on the day of induction and 48 hours later (total amount will be 0.150+0.150=0.300 μg/mouse in 0.2 ml dosage volume).

Group Assignment

The mice were allocated to the following treatment groups (table 13):

TABLE 13

| | Treatment | Administration |
|---|---|---|
| Group | Groups | schedule |
| 1 | Negative Control - purified water (oral) | Daily for 30 days |
| 2 | GA blocking - 12.5 mg/kg (subcutaneous | Once |
| | along with encephalitogen) | |
| 3 | GA - 250 mg/kg (subcutaneous) | GA - daily for 10 |
| | | days |
| 4 | GA - 250 mg/kg (subcutaneous) + | GA - daily for 10 |
| | LAQ - 2.5 mg/kg (oral) | days + LAQ - |
| | | daily for 30 days |
| 5 | GA - 250 mg/kg (subcutaneous) + | GA - daily for 10 |
| | LAQ 5.0 mg/kg (oral) | days + LAQ - |
| | | daily for 30 days |
| 6 | LAQ - 2.5 mg/kg | LAQ - daily for 30 |
| | | days |
| 7 | LAQ - 5.0 mg/kg | LAQ - daily for 30 |
| | | days |
| 8 | LAQ - 25.0 mg/kg | LAQ - daily for 30 |
| | | days |

Preparation and Administration of Encephalitogenic Emulsion

Oil Portion:

CFA (containing 1 mg/ml MT) was enriched to the concentration of 2 mg/ml: 33.5 mg/MT were added to 33.5 ml CFA.

Liquid Portion:

Groups 1, 3-8: 45.9 mg MOG was diluted in 15.3 ml Normal Saline (3.0 mg/ml MOG stock solution). 12.5 mL of 3.0 mg/ml stock solution was diluted in 12.5 mL Normal Saline to yield 1.5 mg MOG/mL. Group 2: 5 mg/mL stock solution of GA were prepared in Normal Saline. 2.5 mL of this solution was mixed with 2.5 mL of 3 mg/ml MOG stock solution to yield 1.5 mg MOG/mL and 2.5 mg GA/mL. The emulsification was made from equal parts of oil and liquid portions (1:1) in two syringes connected to each other with Leur lock, transferred to insulin syringe and 0.2 ml was injected the right flank of each mouse. The dose of the MOG in all the groups (1 to 8) was 150 mg/mouse. The dose of the GA in group 2 was 250 mg/mouse.

Preparation and Administration of Pertussis Toxin

60 mL Pertussis toxin (200 mg/ml) was added to 31.940 ml saline to yield about 375 ng/ml. The pertussis toxin was administered intraperitoneally on the day of encephalitogen injection and 48 hours later (75.0 ng/0.2 ml/mouse).

Preparation and Administration of LAQ Formulations:

The test concentrations of laquinimod were prepared in purified water. For high dose (25.0 mg/kg) 2.5 mg/mL stock solution was prepared (group 8). The 2.5 mg/mL stock solution was diluted 1:5 and 1:10 to yield 0.5 and 0.25 mg/mL for dose levels of 5.0 mg/kg for groups 5 and 7 and 2.5 mg/kg respectively for groups #4 and 6. Laquinimod was administered daily from the initiation of the study, to the respective groups by gavage at a volume of 0.2 ml/mouse.

Glatiramer Acetate:

A 50.0 mg/ml stock solution of GA was prepared in Saline. 5.0 mL of 50.0 mg/mL GA was aliquoted in 10 tubes and stored at −20° C. Every day one tube was thawed and brought to RT. GA was administered subcutaneously daily for 10 days from the initiation of the study to the mice of groups 3, 4 and 5 at a volume of 0.1 mL.

### Experimental Observations

Morbidity and Mortality

All animals were examined once daily to detect if any is dead or moribund.

Clinical Signs

Scoring of EAE clinical signs was started from Day 10 post EAE induction and was continued daily until Day 30. The clinical signs were recorded according to a grading system described in the Table 2 above.

All mice having scores of 1 and above were considered sick. When the first clinical sign appeared all mice were given food soaked in water, which was spread on different places on the bedding of the cages. An animal which continued to have score 5 for three days was killed on humane grounds and given score 6 on the next day. For calculation purposes, the score of animals that were sacrificed or died (6) was carried forward.

Interpretation of Results

Same as in Example 2.4.

Results

A summary of the incidence, mortality, MMS, GMS, duration of the disease, onset of the disease and the activity of each group according to Incidence, MMS and GMS is shown in the summarized Table 14.

Clinical Signs and Mortality

No clinical symptoms were observed during the first 10 days of the study (before initiation of scoring for EAE). There was no morbidity and mortality prior to onset of the EAE disease. One mouse died in group treated with 2.5 mg/kg laquinimod. No mortality was observed in the other treated groups.

Incidence, Onset and Duration of Disease

The incidence of disease in the vehicle treated negative control group was 14/15 compared to 6/15 in the GA blocking positive control group. The onset and duration of disease were 14.9±4.9 and 16.1±4.9 respectively in the vehicle treated negative control group compared to 26.1±7.0 and 12.1±5.3 respectively in the GA blocking positive control group.

Mean Maximal Score (MMS) and Group Mean Score (GMS)

The MMS of the vehicle treated negative control group was 3.4+1.1 and of GA blocking positive control group was 1.3+1.8. According to GMS, the GA blocking positive control (12.5 mg/kg) and laquinimod (25 mg/kg) treated groups suppressed EAE compared to the negative control group by 66.7% (p=0.0004) and 100.0% (p>0.000001) respectively. GA administered subcutaneously exhibited no suppressive activity −4.8% (p=0.7). Group treated with laquinimod (5 mg/kg) along with GA at dose level of 250 mg/kg (subcutaneous) exhibited additive effect −81.0% activity according to GMS (p=0.000001) which was not significant because of high activity exhibited by laquinimod alone −71.4% (p=0.00001) according to GMS). GA administered subcutaneously exhibited no suppressive activity −4.8% (p=0.7). Group treated with laquinimod (2.5 mg/kg) along with GA at dose level of 250 mg/kg (subcutaneous) exhibited no additive effect −76.2% activity according to GMS.

Conclusions

The test was valid as both calculated parameters of the negative control group, the incidence (93.3%) and Mean Maximal Score (3.4) met with the acceptance criteria. In the present study, the group treated with laquinimod (2.5 mg/kg) did not exhibit any additive effect with GA at dose level of 250 mg/kg (subcutaneous). The additive effect was not significant in the group treated with laquinimod (5 mg/kg) along with GA administered subcutaneously at dose level of 250 mg/kg. (81.0% activity compared to 71.4% activity exhibited by laquinimod (5 mg/kg) alone.

TABLE 14

| | | | | | | | | Mean |
|---|---|---|---|---|---|---|---|---|
| | | | | | | | | Duration |
| | | | | MMS | | GMS | | (days) |
| | | | % | MMS | % | GMS | % | Onset |
| Treatment | Mortality | Incidence | Inhibition 1 | value | Inhibition 2 | value | Inhibition 3 | (days) |
| Negative Control - | 0/15 | 14/15 | — | 3.4 ± 1.1 | — | 2.1 ± 0.9 | — | 16.1 ± 4.9 |
| purified water (oral) | | | | | | | | 14.9 ± 4.9 |
| GA blocking - 12.5 | 0/15 | 6/15 | 57.1% | 1.3 ± 1.8 | 61.8% | 0.7 ± 1.0 | 66.7% | 12.1 ± 5.3 |
| mg/kg | | | | | | | (p = 0.00041) | 26.1 ± 7.0 |
| (subcutaneous along | | | | | | | | |
| with encephalitogen) | | | | | | | | |
| GA - 250 mg/kg | 0/15 | 13/15 | 100.0% | 3.3 ± 1.4 | 2.9% | 2.0 ± 1.0 | 4.8% | 12.1 ± 5.3 |
| (subcutaneous) | | | | | | | (p = 0.70) | 18.1 ± 5.5 |
| GA - 250 mg/kg | 0/15 | 9/15 | 35.7% | 1.8 ± 1.7 | 47.1% | 0.5 ± 0.9 | 76.2% | 27.3 ± 5.0 |
| (subcutaneous) + | | | | | | | (p = 0.00005) | 3.7 ± 5.0 |
| _LAQ - 2.5 mg/kg | | | | | | | | |
| (oral) | | | | | | | | |
| GA - 250 mg/kg | 0/15 | 8/15 | 42.9% | 1.8 ± 1.8 | 47.4% | 0.4 ± 0.6 | 81.0% | 28.3 ± 3.6 |
| (subcutaneous) + | | | | | | | (p = 0.00001) | 2.7 ± 3.6 |
| LAQ 5.0 mg/kg | | | | | | | | |
| (oral) | | | | | | | | |
| LAQ - 2.5 mg/kg | 1/15 | 6/15 | 57.1% | 1.5 ± 2.1 | 55.9% | 0.5 ± 0.8 | 76.2% | 0.8 ± 2.6 |
| | | | | | | | (p = 0.00003) | 30.3 ± 2.6 |
| LAQ - 5.0 mg/kg | 0/15 | 9/15 | 64.3% | 2.1 ± 1.8 | 38.2% | 0.6 ± 0.7 | 71.4% | 5.7 ± 6.6 |
| | | | | | | | (p = 0.00001) | 25.1 ± 6.8 |
| LAQ - 25.0 mg/kg | 0/15 | 0/15 | 100.0% | 0.0 ± 0.0 | 100.00% | 0.0 ± 0.0 | 100.00% | 31.0 ± 0.0 |
| | | | | | | | (p > 0.00001) | 0.0 ± 0.0 |

Summary of Group Mean Score (GMS) Inhibition in MOG Combination Studies (Examples 2.1-2.5)

TABLE 15

| | | | LQ | LQ | LQ | | | |
|---|---|---|---|---|---|---|---|---|
| | GA- | | 2.5 | 5 | 25 | +2.5 | +5 | +25 |
| | Block | GA s.c | mg/kg | mg/kg | mg/kg | mg/kg | mg/kg | mg/kg |
| Study 1 | 48 | | | 31.2 | 54.4 | | 71.3 | 80.4 |
| Study 2 | 77.5 | | 53.7 | 68.2 | | 60.8 | 78.4 | |
| Study 3 | 78.9 | | | 70 | 95.1 | | 89.5 | 100 |
| Study 4 | (76.2) | 38.1 | | 71.4 | 95.2 | | 85.7 | 95.2 |
| Study 5 | (66.7) | 4.8 | 76.2 | 71.4 | 100 | 76.2 | 81 | |

### Example 2.6

### Combination Study in Biozzi Mice

The objective of this study was to determine the optimal effective dose of laquinimod and to study the suppressive effect of laquinimod and GA alone and together, following daily administration by oral gavage, once a day for 59 days in relapsing remitting (RR) EAE in Biozzi mice (preventive model).

EAE is an animal model for multiple sclerosis. In the Biozzi AB/H EAE mice, a RR EAE can be induced. In this model, unlike the Lewis rat model and many other acute EAE models in mice, the mouse does not become refractory to disease induction following a single acute episode. Re-injection of an encephalitogen can induce a chronic relapsing disease that enables measuring long-term effects on subsequent relapses rather than immediate effects.

Materials

Glatiramer acetate drug substance; laquinimod drug substance; Lyophilized Mouse spinal cord homogenate (MSCH) from ICR mice; Incomplete Freund's Adjuvant (ICFA) “Difco”; Milli-Q purified water (pH2O); Mycobacterium tuberculosis, H37Ra (MT); and Difco PBS, “Sigma”.

### Experimental Animals

Healthy, specific pathogen free, female Biozzi mice were used in the study. The mice were 6-11 weeks old at time of induction, and weighed 20 g±15%.

Test Procedure

EAE Induction:

The EAE induction was performed according to procedure “RR-EAE.Biozzi” The Biozzi mice were weighed and injected subcutaneously and re-injected one week later with encephalitogenic agent (MSCH emulsion). 0.3 mL of the encephalitogenic agent containing 1.0 mg of MSCH and 200.0 μg of MT was injected subcutaneously, at two sites in the right flank of each mouse (0.15 mL at each site). The second injection was done in a similar manner in the left flank, one week later.

Treatment Group Assignment:

On the day of first injection of encephalotigen, the Biozzi mice were randomly allocated to eight treatment groups as follows: Tables 16-17.

TABLE 16

| | | Administration |
|---|---|---|
| Group | Group Identification | schedule |
| 1 | 25.0 mg/kg LAQ + 12.5 mg/kg | Daily from injection of |
| | GA administered orally | encephalitogen for 59 days |
| 2 | 5.0 mg/kg LAQ + 12.5 mg/kg | Daily from injection of |
| | GA administered orally | encephalitogen for 59 days |
| 3 | 25.0 mg/kg LAQ administered | Daily from injection of |
| | orally | encephalitogen for 59 days |
| 4 | 12.5 mg/kg LAQ administered | Daily from injection of |
| | orally | encephalitogen for 59 days |
| 5 | 5.0 mg/kg LAQ administered | Daily from injection of |
| | orally | encephalitogen for 59 days |
| 6 | GA 12.5 mg/kg administered | Daily from injection of |
| | orally | encephalitogen for 59 days |
| 7 | Control (purified water) | Daily from injection of |
| | | encephalitogen for 59 days |

TABLE 17

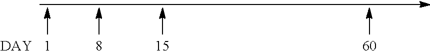

| DAY | TEST PROCEDURE |
|---|---|
| 1 | Subcutaneous injection of MSCH + CFA emulsion into right flank |
| 1 | Allocation of mice to treatment groups and initiation of |
| | treatment for 59 consecutive days |
| 8 | Subcutaneous injection of MSCH + CFA emulsion into left flank |
| 15 | Initiation of scoring of mice for EAE clinical signs |
| 60 | Termination of study |

Schedule of Procedures During Study

Preparation of Test Formulations

The working concentrations of the laquinimod and GA were prepared in purified water.

A 5.0 mg/mL stock solution of laquinimod and a 1.25 mg/mL stock solution of GA was prepared. The 5.0 mg/mL laquinimod stock solution was diluted 1:2, 1:4 and 1:10 to yield 2.5, 1.25 and 0.5 mg/mL for dose levels of 25.0 mg/kg, 12.5 mg/kg and 5.0 mg/kg laquinimod respectively. The 2.5 mg/mL GA stock solution was diluted 1:2, to yield 1.25 mg/mL for dose level of 12.5 mg/kg GA. The 2.5 mg/mL GA stock solution was diluted 1:2 with 5.0 mg/mL laquinimod to yield 1.25 mg/mL GA and 2.5 mg/mL laquinimod for dose levels of 12.5 mg/kg GA and 25 mg/kg laquinimod. The 5.0 mg/mL laquinimod stock solution was diluted 1:5 to yield 1 mg/mL. The 2.5 mg/mL GA stock solution was diluted 1:2 with 1.0 mg/mL laquinimod to yield 1.25 mg/mL GA and 0.5 mg/mL laquinimod for dose levels of 12.5 mg/kg GA and 5.0 mg/kg laquinimod.

Test Article Administration

The mice in all the groups were administered daily by oral gavage a volume dose level of 250 mL/mouse of the respective test formulations from the day the mice are allocated to the treatment groups (first injection of encephalitogen) for 59 consecutive days until the termination of the study.

Clinical Observation and Scoring

The mice were examined and scored according to the following table, until the termination of the study (60 days after initiation of treatment).

TABLE 18
Evaluation of the EAE clinical signs.

| Scale | Signs | Description |
|---|---|---|
| 0 | Normal behavior | No neurological signs. |
| 1 | Tail weakness | The mouse tail is limp and droops. |
| 2 | Righting reflex | Limp tail with decrease in righting reflex |
| 3 | Hind legs weakness | Limb paresis, wobbly walk - when the |
| | | mouse walks the hind legs are unsteady |
| | | (ataxia). |
| 4 | Hind legs paralysis | The mouse can't move it's hind legs and it |
| | | drags them when he walks. |
| 5 | Full paralysis | The mouse can't move it's legs at all, it |
| | (moribund) or death | looks thinner and emaciated. |

*All mice having score 5 for more than three days were sacrificed on humane grounds. The score of 5 was carried forward. All mice having scores of 1 and above were considered sick.

Interpretation of Results

The activity of the test articles for the first and second phase of RR EAE was calculated by comparing the incidence of disease, mortality, onset and duration of disease, group mean score and mean maximal score to the control group. Calculations were carried separately and together for the first and the second relapse. The calculations are as shown in Example 2.4.

Results

A summary of the incidence, mean maximal score, group mean score/individual mean scores and mean duration of disease during first attack, second attack and first and second attack together are shown in the summary tables 19-21.

During the observation period, 2 and 3 mice died, respectively in groups treated with GA (12.5 mg/kg) and water (Control).

During the first and second attacks, and when the scores of the first and the second attacks of EAE were calculated together, groups treated with dose level of 12.5 mg/kg GA along with laquinimod at dose levels of 25.0 and 5.0 mg/kg were the most effective in the suppression of EAE.

During the first attack, the individual mean scores of these groups were 82.0% and 66.3% lower than for the water administered control group (see table 20). During the second attack the individual mean scores of these groups were 84.6% and 64.5% lower than for the water administered control group (see table 21).

When the scores of the first and the second attacks of EAE were calculated together, the group mean score of these groups were 82.6% and 60.9% lower than for the water administered control group (see table 19).

Groups treated with laquinimod alone were more effective in the suppression of EAE during the first relapse than during the second relapse.

During the first attack, groups treated with 5, 12.5 and 25.0 mg/kg laquinimod were 38.2%, 53.3% and 42.8% lower than for the water administered control group (see table 20). During the second attack, groups treated with 5, 12.5 and 25.0 mg/kg laquinimod were 18.4%, 41.4% and 29.2% lower than for the water administered control group (see table 21).

5.6 GA administered alone at dose level of 12.5 mg/kg was 15.6%, 35.5% and 21.7% lower than for the water administered control group during the first, second and first and second attacks together respectively.

TABLE 19

| | | Incidence | | MMS | | GMS | | |
|---|---|---|---|---|---|---|---|---|
| | | | % | | % | | % | Mean |
| Treatment | Mortality | | inhibition | value | inhibition | value | inhibition | Duration(Days) |
| 25.0 mg/kg | 0/14 | 3/14 | 76.9% | 1.1 ± 1.9 | 69.4% | 0.4 ± 0.8 | 82.6% | 5.3 ± 12.3 |
| laquinimod + | | | | | | | p = 0.0005 | |
| 12.5 mg/kg GA | | | | | | | | |
| 5.0 mg/kg | 0/14 | 7/14 | 46.2% | 1.9 ± 2.0 | 47.2% | 0.9 ± 1.0 | 60.9% | 14.6 ± 6.2 |
| laquinimod + | | | | | | | p = 0.007 | |
| 12.5 mg/kg GA | | | | | | | | |
| 25.0 mg/kg | 0/14 | 9/14 | 30.8% | 2.6 ± 2.0 | 27.8% | 1.6 ± 1.3 | 30.4% | 23.0 ± 16.4 |
| laquinimod | | | | | | | p = 0.19 | |
| 12.5 mg/kg | 0/14 | 10/14 | 23.1% | 2.8 ± 1.8 | 22.2% | 1.3 ± 1.0 | 43.5% | 17.5 ± 14.0 |
| laquinimod | | | | | | | p = 0.05 | |
| 5.0 mg/kg | 0/14 | 10/14 | 23.1% | 3.1 ± 1.7 | 16.2% | 1.8 ± 1.1 | 21.7% | 26.5 ± 17.0 |
| laquinimod | | | | | | | p = 0.36 | |
| Glatimer acetate | 2/14 | 12/14 | 7.7% | 3.6 ± 1.6 | 0.0% | 1.8 ± 1.5 | 21.7% | 23.8 ± 16.2 |
| 12.5 mg/kg | | | | | | | p = 0.43 | |
| Control(Purified | 2/14 | 13/14 | — | 3.6 ± 1.4 | — | 2.3 ± 1.6 | — | 29.1 ± 15.9 |
| water) | | | | | | | | |

TABLE 20

| | | | | | MMS | | IMS | | Mean |
|---|---|---|---|---|---|---|---|---|---|
| | | | | % | | % | | % | Duration |
| No. | Treatment | Mortality | Incidence | Inhibition 1 | value | Inhibition 2 | value | Inhibition 3 | (days) |
| 1 | 25.0 mg/kg | 0/14 | 3/14 | 78.6% | 1.1 ± 1.9 | 69.4% | 12.5 ± 21.9 | 82.0% | 3.0 ± 6.6 |
| | LAQ + 12.5 | | | | | | | p = 0.01 | |
| | mg/kg GA | | | | | | | | |
| 2 | 5.0 mg/kg | 0/14 | 7/14 | 46.2% | 1.9 ± 2.0 | 47.2% | 23.4 ± 26.6 | 66.3% | 8.4 ± 9.6 |
| | LAQ + 12.5 | | | | | | | P = 0.04 | |
| | mg/kg GA | | | | | | | | |
| 3 | 25.0 mg/kg | 0/14 | 9/14 | 30.8% | 2.6 ± 2.0 | 27.8% | 39.7 ± 32.3 | 42.8% | 13.9 ± 10.0 |
| | LAQ | | | | | | | P = 0.18 | |
| 4 | 12.5 mg/kg | 0/14 | 10/14 | 23.1% | 2.7 ± 1.9 | 25.0% | 32.4 ± 24.9 | 53.3% | 9.6 ± 8.6 |
| | LAQ | | | | | | | P = 0.23 | |
| 5 | 5.0 mg/kg | 0/14 | 10/14 | 23.1% | 2.9 ± 1.9 | 19.4% | 42.9 ± 29.8 | 15.6% | 15.4 ± 10.9 |
| | LAQ | | | | | | | P = 0.69 | |
| 6 | GA 12.5 | 2/14 | 12/14 | 7.7% | 3.5 ± 1.6 | 2.8% | 58.6 ± 70.5 | | 15.7 ± 14.0 |
| | mg/kg | | | | | | | | |
| 7 | Control | 3/14 | 13/14 | — | 3.6 ± 1.4 | — | 69.4 ± 74.3 | — | 18.8 ± 14.4 |
| | (purified | | | | | | | | |
| | water) | | | | | | | | |

TABLE 21

| | | | | | MMS | | IMS | | Mean |
|---|---|---|---|---|---|---|---|---|---|
| | | | | % | | % | | % | Duration |
| No. | Treatment | Mortality | Incidence | Inhibition 1 | value | Inhibition 2 | value | Inhibition 3 | (days) |
| 1 | 25.0 mg/kg | 0/14 | 2/14 | 80.3% | 0.6 ± 1.5 | 79.3% | 7.3 ± 19.8 | 84.6% | 2.3 ± 5.9 |
| | LAQ + 12.5 | | | | | | | p = 0.002 | |
| | mg/kg GA | | | | | | | | |
| 2 | 5.0 mg/kg | 0/14 | 6/14 | 41.0% | 1.5 ± 1.8 | 48.3% | 16.8 ± 23.3 | 64.5% | 6.1 ± 8.9 |
| | LAQ + 12.5 | | | | | | | p = 0.02 | |
| | mg/kg GA | | | | | | | | |
| 3 | 25.0 mg/kg | 0/14 | 8/14 | 21.5% | 2.3 ± 2.1 | 20.7% | 33.5 ± 32.2 | 29.2% | 9.1 ± 8.6 |
| | LAQ | | | | | | | p = 0.33 | |
| 4 | 12.5 mg/kg | 0/14 | 9/14 | 11.6% | 2.5 ± 2.0 | 13.8% | 27.7 ± 23.1 | 41.4% | 7.9 ± 6.5 |
| | LAQ | | | | | | | p = 0.12 | |
| 5 | 5.0 mg/kg | 0/14 | 10/14 | 1.8% | 2.8 ± 1.8 | 3.4% | 38.6 ± 28.5 | 18.4% | 11.4 ± 11.5 |
| | LAQ | | | | | | | p = 0.52 | |
| 6 | GA 12.5 | 0/12 | 6/12 | 31.2% | 2.0 ± 1.7 | 31.0% | 30.5 ± 33.0 | 35.5% | 9.4 ± 10.5 |
| | mg/kg | | | | | | | p = 0.25 | |
| 7 | Control | 0/11 | 8/11 | — | 2.9 ± 1.9 | — | 47.3 ± 37.2 | — | 13.2 ± 9.9 |
| | (purified | | | | | | | | |
| | water) | | | | | | | | |

### Example 2.7

### MSCH-Induced EAE in CSJL Mice

The objective of this study was to compare the suppressive activity of laquinimod following daily administration by oral gavage alone and along with GA in the EAE model in CSJL/FI mice.

MS is an immune-mediated disorder of the CNS leading to progressive decline of motor and sensory functions causing permanent disability. The CSJL/FI strain of mouse was selected, as it is an established EAE model to test for the efficacy of candidate molecules for the treatment of MS.

General Design

Disease was induced in all mice by the injection of the encephalitogenic emulsion (MSCH/CFA). The test articles and the vehicle were administered orally twice a day.

Materials

Glatiramer acetate; Laquinimod; Purified water (RO water); Pertusis toxin, “Sigma”; Lyophilized mouse spinal cord homogenate (MSCH); and Complete Freund's Adjuvant (CFA) “Sigma”.

### Experimental Animals

Healthy, nulliparous, non-pregnant female mice of the CSJL/FI strain weighting about 17-20 g on arrival were approximately 10 weeks of age. The body weights of the animals were recorded on the day of delivery. Overtly healthy animals were assigned to study groups arbitrarily before treatment commences.

Test Procedures

Group Assignment

The mice were allocated to the following eight treatment groups on Table 22:

TABLE 22

| | | | | Administration |
|---|---|---|---|---|
| Group | Group Identification | Dose/day | | schedule |
| 1 | Vehicle | 10 | mL/kg | Oral |
| 2 | GA Blocking | 0.5 | mg/kg | Along with |
| | (sub-optimal dose) | | | encephalitogen |
| 3 | LAQ | 1.0 | mg/kg | Oral |
| | (Positive control) | | | |
| 4 | LAQ | 0.3 | mg/kg | Oral |
| 5 | LAQ | 0.1 | mg/kg | Oral |
| 6 | LAQ | 0.01 | mg/ka | Oral |

| | | | |
|---|---|---|---|
| 7 | LAQ + GA Blocking | 0.1 mg/kg | LAQ oral + GA |
| | (sub-optimal dose) | LAQ + | along with |
| | | 0.5 mg/kg GA | encephalitogen |
| 8 | LAQ + GA Blocking | 0.01 mg/kg | LAQ oral + GA |
| | (sub-optimal dose) | LAQ + | along with |
| | | 0.5 mg/kg GA | encephalitogen |

Preparation and Administration of Encephalitogenic Emulsion

Oil Portion:

CFA (containing 1 mg/ml MT).

Liquid Portion:

For groups 1 and 3 to 6, 160.2 mg MSCH was suspended in 4.0 nil PBS to yield 40 mg/ml MSCH suspension. For groups 2, 7 and 8, GA will be dissolved in PBS to yield 0.4 mg/mL GA/ml. 60.0 mg MSCH will be suspended in 1.5 ml of 0.4 mg/ml GA solution to yield 40 mg/ml MSCH suspension in 0.4 mg/ml GA solution.

The emulsion was made from equal parts of oil and liquid portions in two syringes connected to each other with Leur lock, transferred to insulin syringe.

0.05 ml was injected into the left foot-pad of each mouse.

The concentration of MSCH in emulsion in all groups was 20 mg/mL. The dose of the MSCH in all the groups was 1.0 mg/0.05 ml/mouse. The concentration of MT in emulsion in all groups was 0.5 mg/mL. The dose of the MT in all the groups was 0.025 mg/0.05 ml/mouse. The concentration of GA in emulsion in all groups was 0.2 mg/mL. The dose of the GA in groups 2, 7 and 8 was 10 mg/0.05 ml/mouse equivalent to 0.5 mg/kg. This is the sub therapeutic dose which is 25 times lower than the therapeutic dose which is 12.5 mg/kg (250 mg/mouse).

Preparation and Administration of Pertussis Toxin

70 mL Pertussis toxin (200 mg/ml) was added to 69.93 ml PBS to yield 200 ng/ml. The pertussis toxin was administered intravenous on the day of encephalitogen injection and 48 hours later (100.0 ng/0.5 ml/mouse).

Preparation and Administration of Working Concentrations of Laquinimod

Solutions at concentration of 0.001, 0.01, 0.03 and 0.1 mg/mL laquinimod were prepared in water for dose levels of 0.01, 0.1, 0.3 and 1.0 mg/kg respectively. The test formulations were stored at 2 to 8° C. until use in amber colored bottles. The mice were administered with the respective dose levels of laquinimod at volume dose level of 200 mL/mouse. The test formulations were vortexed before dispensing in syringe. The test article formulations were administered to the respective groups by oral gavage. The vehicle (purified water) was administered to the negative control group (Group 1) and to group 2 in a similar manner. The different dose levels laquinimod were administered to the respective groups once a day.

### Experimental Observations

Morbidity and Mortality

All animals were examined once daily to detect if any is dead or moribund.

Clinical Signs

Scoring of EAE clinical signs was initiated from the 10th day post-EAE induction and was continued daily until Day 23. The clinical signs were recorded according to a grading system described in the table 23 below.

TABLE 23

| Score | Signs | Description |
|---|---|---|
| 0 | Normal behavior | No neurological signs. |
| 1 | Tail weakness | The mouse tail is limp and droops. |
| 2 | Hind legs weakness | Limb paresis, wobbly walk - when the |
| | | mouse walks the hind legs are unsteady. |
| 3 | Hind legs paralysis | The mouse can't move it's hind legs and it |
| | | drags them when he walks. |
| 4 | Full paralysis | The mouse can't move it's legs at all. it |
| | | looks thinner and emaciated. |
| 5 | Death | |

All mice having scores of 1 and above were considered sick. Animals with score 4 for more than three days were given score 5 and sacrificed for humane reasons. For calculation purposes, the score of animals that are sacrificed or died were carried forward.

Interpretation of Results

The calculations are as shown in Example 2.4.

Results

A summary of the incidence, mortality, MMS, GMS, duration of the disease, onset of the disease and the activity of each group according to Incidence, MMS and GMS is shown in the summarized Table 24.

Clinical Signs and Mortality

Severe EAE clinical signs were observed resulting in mortality of all 10 mice of the control group by Day 16 of the study. Between 0 and 8 mice died in the other treatment groups. Due to severe disease, the efficacy of laquinimod was less than that seen in previous studies.

Incidence, Onset and Duration of Disease

The incidence of disease in the vehicle treated negative control group was 10/10. 90 to 100% incidence was observed in the groups treated with different dose levels of laquinimod alone. 7/10 mice were sick in the group treated with suboptimal dose of GA (0.5 mg/kg) administered along with encephalitogen.

When laquinimod (0.1 mg/kg) was administered to the group treated with sub optimal dose of GA (0.5 mg/kg) the incidence was 6/10 (40% activity). The onset and duration of disease in this group were 18.8±4.9 and 4.1±4.5 respectively compared to 12.2±0.8 and 11.8±0.8 respectively in the vehicle treated negative control group.

Mean Maximal Score (MMS) and Group Mean Score (GMS)

The MMS of the vehicle treated negative control group was 5.0±0.0 as all the mice died. Compared to 1.7±1.6 in the group where laquinimod (0.1 mg/kg) was administered to the group treated with sub optimal dose of GA (66% activity according to MMS). According to GMS, this group suppressed EAE compared to the negative control group by 80.9% (p<0.000001).

Amongst the groups treated with different dose levels of laquinimod, dose level of 1.0 mg/kg was the most effective with 38.1% activity (p=0.006) compared to the control group according to GMS. Group treated with sub optimal dose of GA exhibited 61.9% activity (p=0.0002). When laquinimod (0.01 mg/kg) was administered to the group treated with sub optimal dose of GA, 57.1% activity (p=0.000001). according to GMS was observed compared to the negative control group.

Conclusions

Under the conditions of the test, laquinimod at dose level of 1.0 mg/kg administered daily orally by gavage exhibits additive effect with sub optimal dose of GA (0.5 mg/kg) and is more effective in the suppression of chronic MOG induced EAE in C57Bl mice than administration of laquinimod (1.0 mg/kg) or alone sub optimal dose of GA (0.5 mg/kg).

TABLE 24

| | | | | MMS | | GMS | | Mean | |
|---|---|---|---|---|---|---|---|---|---|
| | | | % | | % | | % | Duration | Onset |
| Treatment | Mortality | Incidence | Inhibition 1 | value | Inhibition 2 | value | Inhibition 3 | (days) | (days) |
| Vehicle (purified | 10/10 | 10/10 | — | 5.0 ± 0.0 | — | 4.2 ± 0.3 | — | 11.8 ± 0.8 | 12.2 ± 0.8 |
| water) | | | | | | | | | |
| GA Blocking - | 3/10 | 7/10 | 30.0% | 2.9 ± 2.1 | 42.0% | 1.6 ± 1.5 | 61.9% | 5.8 ± 4.5 | 17.0 ± 4.9 |
| 0.5 mg/kg (sub- | | | | | | | (p = 0.00003) | | |
| optimal dose) | | | | | | | | | |
| LAQ (1.0 mg/kg) | 3/10 | 10/10 | 0.0% | 3.6 ± 0.0 | 28.0% | 2.6 ± 1.1 | 38.1% | 10.7 ± 1.9 | 13.0 ± 2.0 |
| | | | | | | | (p = 0.0002) | | |
| LAQ (0.3 mg/kg) | 6/10 | 10/10 | 0.0% | 4.2 ± 1.0 | 16.0% | 3.0 ± 1.2 | 28.6 | 10.8 ± 2.0 | 13.2 ± 2.0 |
| | | | | | | | (p = 0.006) | | |
| LAQ (0.1 mg/kg) | 7/10 | 9/10 | 10.0% | 4.1 ± 1.7 | 18.0% | 2.9 ± 1.5 | 31.0% | 9.8 ± 4.0 | 14.2 ± 4.0 |
| | | | | | | | (p = 0.016) | | |
| LAQ (0.01 mg/kg) | 8/10 | 10/10 | 0.0% | 4.8 ± 0.4 | 4.0% | 3.5 ± 0.9 | 16.7 | 11.5 ± 0.8 | 12.5 ± 0.8 |
| | | | | | | | (p = 0.029) | | |
| GA Blocking (0.5 | 2/10 | 10/10 | 0.0% | 3.3 ± 0.9 | 34.0% | 1.8 ± 1.0 | 57.1% | 8.3 ± 2.6 | 15.3 ± 2.2 |
| mg/kg) + LAQ | | | | | | | (p = 0.000001) | | |
| (0.01 mg/kg) | | | | | | | | | |
| GA Blocking (0.5 | 0/11 | 6/10 | 40.0% | 1.7 ± 1.6 | 66.0% | 0.8 ± 1.9 | 80.9 | 4.1 ± 4.5 | 18.8 ± 4.9 |
| mg/kg) + LAQ | | | | | | | (p < 0.0000001) | | |
| (0.1 mg/kg) | | | | | | | | | |

### Example 2.8

### MSCH-Induced EAE in CSJL/FI Mice

The objective of this study was to compare the suppressive activity of laquinimod following daily administration by oral gavage alone and along with GA in the EAE model in CSJL/FI mice.

MS is an immune-mediated disorder of the CNS leading to progressive decline of motor and sensory functions causing permanent disability. The CSJL/FI strain of mouse was selected, as it is an established EAE model to test for the efficacy of candidate molecules for the treatment of MS.

General Design

Disease was induced in all mice by the injection of the encephalitogenic emulsion (MSCH/CFA). The test articles and the vehicle were administered orally twice a day.

Materials

Glatiramer acetate; Laquinimod; Purified water (RO water); Pertusis toxin, “Sigma” Lyophilized mouse spinal cord homogenate (MSCH); Complete Freund's Adjuvant (CFA) “Sigma”; Incomplete Freund's Adjuvant (ICFA), Difco; and PBS “Sigma”.

### Experimental Animals

Healthy, nulliparous, non-pregnant female mice of the CSJL/FI strain weighed about 17-20 g on arrival, and were approximately 9 weeks of age. The body weights of the animals were recorded on the day of delivery.

Overtly healthy animals were assigned to study groups arbitrarily before treatment commenced.

Test Procedures

Group Assignment

The mice were allocated to the following eight treatment groups on Table 25:

| | | | | Administration |
|---|---|---|---|---|
| Group | Treatment Groups | Dose/Day | | Route |
| 1 | Vehicle (purified water) | 10 | mL/kg | Oral |
| 2 | GA Blocking | 0.5 | mg/kg | Along with |
| | (sub-optimal dose) | | | encephalitogen |
| 3 | LAQ | 1.0 | mg/kg | Oral |
| | (Positive control) | | | |
| 4 | LAQ | 0.3 | mg/kg | Oral |
| 5 | LAQ | 0.1 | mg/kg | Oral |
| 6 | LAQ | 0.01 | mg/ka | Oral |

| | | | |
|---|---|---|---|
| 7 | LAQ + GA Blocking | 0.1 mg/kg | LAQ oral + GA |
| | (sub-optimal dose) | LAQ + | along with |
| | | 0.5 mg/kg GA | encephalitogen |
| | LAQ + GA Blocking | 0.01 mg/kg | LAQ oral + GA |
| | (sub-optimal dose) | LAQ + | along with |
| | | 0.5 mg/kg GA | encephalitogen |

Preparation and Administration of Encephalitogenic Emulsion

Oil Portion:

CFA (containing 1 mg/ml MT)+ICFA in ratio 1:2 to yield 0.5 mg/mL Mycobacterium tuberculosis.

Liquid Portion:

For groups 1 and 3 to 6, 360.0 mg MSCH was suspended in 3.0 ml PBS to yield 120 mg/ml MSCH suspension. For groups 2, 7 and 8, GA will be dissolved in PBS to yield 0.4 mg/mL GA/ml. 180.0 mg MSCH was suspended in 1.5 ml of 0.4 mg/ml GA solution to yield 120 mg/ml MSCH suspension in 0.4 mg/ml GA solution.

The emulsion was made from equal parts of oil and liquid portions in two syringes connected to each other with Leur lock, transferred to insulin syringe.

0.05 ml was injected into the left foot-pad of each mouse. The concentration of MSCH in emulsion in all groups was 60 mg/mL. The dose of the MSCH in all the groups was 3.0 mg/0.05 ml/mouse. The concentration of MT in emulsion in all groups was 0.25 mg/mL. The dose of the MT in all the groups was 0.0125 mg/0.05 ml/mouse. The concentration of GA in emulsion in all groups was 0.2 mg/mL.

The dose of the GA in groups 2, 7 and 8 was 10 μg/0.05 ml/mouse equivalent to 0.5 mg/kg. This is the sub therapeutic dose which is 25 times lower than the therapeutic dose which is 12.5 mg/kg (250 μg/mouse).

Preparation and Administration of Pertussis Toxin

36 μL Pertussis toxin (200 μg/ml) was added to 44.964 ml PBS to yield 160 ng/ml. The pertussis toxin was administered intravenous on the day of encephalitogen injection and 48 hour later (80.0 ng/0.5 ml/mouse).

Preparation and Administration of Working Concentrations of Laquinimod

Solutions at concentration of 0.001, 0.01, 0.03 and 0.1 mg/mL laquinimod were prepared in water for dose levels of 0.01, 0.1, 0.3 and 1.0 mg/kg respectively. The test formulations were stored at 2 to 8° C. until use in amber colored bottles.

The mice were administered with the respective dose levels of laquinimod at volume dose level of 200 μL/mouse. The test formulations were vortexed before dispensing in syringe.

The test article formulations were administered to the respective groups by oral gavage. The vehicle (purified water) was administered to the negative control group (Group 1) and to group 2 in a similar manner.

The different dose levels laquinimod were administered to the respective groups once a day.

### Experimental Observations

### Morbidity and Mortality

All animals were examined once daily to detect if any is dead or moribund.

Clinical Signs

Scoring of EAE clinical signs was initiated from the 10 day post-EAE induction and was continued daily until Day 23. The clinical signs were recorded on observation cards according to a grading system described in the table 23 above. Animals with score 4 for more than three days were given score 5 and sacrificed for humane reasons. For calculation purposes, the score (5) of animals that are sacrificed or died were carried forward.

Interpretation of Results

The calculations are as shown in Example 2.4.

Results

A summary of the incidence, mortality, MMS, GMS, duration of the disease, onset of the disease and the activity of each group according to Incidence, MMS and GMS is shown in the summarized Table 26.

Clinical Signs and Mortality

Seven mice died in the Vehicle treated control group due to severe EAE clinical signs. Between 0 and 2 mice died in the other treatment groups.

Incidence, Onset and Duration of Disease

The incidence of disease in the vehicle treated negative control group was 11/11. 91% incidence was observed in the laquinimod (0.01 mg/kg) treated group.

Dose dependency in the activity was observed at different dose levels of laquinimod compared to the vehicle treated control group. According to incidence, in the groups treated with laquinimod, at dose levels of 0.01, 0.1, 0.3 and 1.0 mg/kg activity of 9.1%, 27.3%, 36.4%, 90.9% was observed compared to the vehicle treated control group.

4/11 mice were sick in the group treated with suboptimal dose of GA (0.5 mg/kg) administered along with encephalitogen.

When laquinimod (0.1 mg/kg) was administered to the group treated with suboptimal dose of GA (0.5 mg/kg) the incidence was 0/11 (100% activity) indicating some additive effect.

However when laquinimod (0.01 mg/kg) was administered to the group treated with sub optimal dose of GA (0.5 mg/kg) the incidence was 4/11 which was similar to GA (0.5 mg/kg) administered alone indicating that the two test articles did not interfere with activity of the other.

Compared to the Control group there was a delay in the onset of the disease and duration of disease at different dose levels of laquinimod which was dose dependent except the lowest dose of laquinimod (0.01 mg/kg) where the duration and onset of disease were 13.7±3.8 and 9.5±3.9 which were similar to those in the control group where the duration and onset of disease were 11.5±2.7 and 11.5±0.7 respectively.

Mean Maximal Score (MMS) and Group Mean Score (GMS)

The MMS of the vehicle treated negative control group was 4.5±0.8.

MMS of 1.1±15 was observed in group treated with sub optimal dose of GA (0.5 mg/kg) administered along with encephalitogen.

In the groups treated with different dose levels of laquinimod, activity of 35.3%, 55.9%, 73.5% and 97.1% was observed at dose levels of 0.01, 0.1, 0.3 and 1.0 mg/kg according to GMS.

According to GMS, group treated with sub optimal dose of GA exhibited 79.4% activity (p=0.0002). When laquinimod (0.1 mg/kg) was administered to the group treated with sub optimal dose of GA, 100.0% activity (p<0.000001 was observed compared to the negative control group.

Conclusions

Under the conditions of the test, dose dependency in activity of laquinimod administered daily, orally by gavage was observed at dose levels of 0.01, 0.1, 0.3 and 1.0 mg/kg.

Laquinimod at 1.0 mg/kg administered daily, orally by gavage exhibits additive effect with suboptimal dose of GA (0.5 mg/kg) and is more effective in the suppression of chronic MOG induced EAE in C57Bl mice (100% activity) than administration of laquinimod −1.0 mg/kg (55.9%) or sub optimal dose of GA −0.5 mg/kg (79.4%) alone.

TABLE 26

| | | | | MMS | | GMS | | Mean | |
|---|---|---|---|---|---|---|---|---|---|
| | | | % | | % | | % | Duration | Onset |
| Treatment | Mortality | Incidence | Inhibition 1 | value | Inhibition 2 | value | Inhibition 3 | (days) | (days) |
| Vehicle (purified | 7/11 | 10/11 | — | 4.5 ± 0.8 | — | 3.4 ± 1.3 | — | 11.5 ± 2.7 | 11.5 ± 0.7 |
| water) | | | | | | | | | |
| GA Blocking - | 0/11 | 4/11 | 60.0% | 1.1 ± 1.5 | 75.5% | 0.7 ± 1.0 | 79.4%( ) | 3.6 ± 5.1 | 20.4 ± 5.1 |
| 0.5 mg/kg (sub- | | | | | | | | | |
| optimal dose) | | | | | | | | | |
| LAQ (1.0 mg/kg) | 0/11 | 1/11 | 90.0% | 0.3 ± 0.9 | 93.3% | 0.1 ± 0.3 | 97.1%( ) | 0.5 ± 1.8 | 23.5 ± 1.8 |
| LAQ (0.3 mg/kg) | 1/11 | 7/11 | 30.0% | 2.1 ± 1.8 | 53.3 | 0.9 ± 0.9 | 73.5%( ) | 4.2 ± 3.7 | 17.3 ± 5.5 |
| LAQ (0.1 mg/kg) | 8/11 | 1/11 | 90.0% | 2.6 ± 1.8 | 42.2 | 1.5 ± 1.2 | 55.9%( ) | 6.9 ± 5.1 | 16.5 ± 5.3 |
| LAQ (0.01 mg/kg) | 2/11 | 10/11 | 0.0% | 3.4 ± 1.4 | 24.4% | 2.2 ± 1.2 | 35.3%( ) | 9.5 ± 3.9 | 13.7 ± 3.8 |
| GA Blocking (0.5 | 0/11 | 0/11 | 100.0% | 0.0 ± 0.0 | 100.0% | 0.0 ± 0.00 | 100.0%( ) | 0.0 ± 0.0 | 24.0 ± 0.0 |
| mg/kg) + LAQ | | | | | | | | | |
| (0.01 mg/kg) | | | | | | | | | |
| GA Blocking (0.5 | 1/11 | 4/11 | 60.0% | 1.3 ± 1.8 | 71.1% | 0.7 ± 1.1 | 79.4%( ) | 3.5 ± 4.8 | 19.8 ± 5.9 |
| mg/kg) + LAQ | | | | | | | | | |
| (0.1 mg/kg) | | | | | | | | | |

Results/Discussion

It is important to note the mouse dosing presented here cannot be used to determine human dosing by simply adjusting for body weight, because a gram of mouse tissue is not equivalent to a gram of human tissue. For this reason, the National Institutes of Health (NIH) provides a table of Equivalent Surface Area Dosage Conversion Factors below (Table 27) which provides conversion factors that account for surface area to weight ratios between species.

TABLE 27
Equivalent Surface Area Dosage Conversion Factors

| | To | | | | | |
|---|---|---|---|---|---|---|
| | | Mouse | Rat | Monkey | Dog | Man |
| | | 20 g | 150 g | 3 kg | 8 kg | 60 kg |
| FROM | Mouse | 1 | ½ | ¼ | ⅙ | 1/12 |
| | Rat | 2 | 1 | ½ | ¼ | 1/7 |
| | Monkey | 4 | 2 | 1 | ⅗ | ⅓ |
| | Dog | 6 | 4 | 1⅔ | 1 | ½ |
| | Man | 12 | 7 | 3 | 2 | 1 |

### Example 3

### Clinical Trial (Phase in)—Assessment of Add-on Effect of Laquinimod in Relapsing Multiple Sclerosis (RMS) Subjects Treated with Glatiramer Acetate (GA) or Interferon-Beta (IFN-β)

A multinational, multicenter, randomized, double-blind, parallel-group, placebo-controlled study, followed by a double-blind active extension phase is conducted to assess the safety, tolerability and efficacy of two daily doses of oral laquinimod (0.6 mg or 1.2 mg) in adjunct to glatiramer acetate (GA) or interferon-beta (IFN-β)-1a/1b preparations in subjects with relapsing multiple sclerosis (RMS).

Study Duration

The total study duration for each eligible subject will be up to 19 months:

Screening phase: up to about 1 month.

Double blind treatment phase: about 9 months of once-daily oral administration of laquinimod 0.6 mg/day, 1.2/day or placebo in addition to current therapy (i.e., subcutaneous GA 20 mg or any of the following IFN-β preparations: Avonex®, Betaseron®/Betaferon®, Rebif® or Extavia®).
Double-Blind Active Extension (DBAE) phase: all subjects who complete all 9 months of the DBPC treatment phase are offered the opportunity to continue to a DBAE phase. During this phase, all subjects continue the same background injectable treatment which they used in the DBPC phase.
Subjects who were originally assigned to either of the active oral treatment arm (laquinimod 0.6 mg or 1.2 mg) continue with their original oral treatment assignment. Subjects originally assigned to placebo are equally randomized to either laquinimod 0.6 mg or 1.2 mg. The duration of this phase is 9 months.

Study Population

Relapsing Multiple Sclerosis (RMS).

Study Design

Eligible subjects are equally (1:1:1) randomized into one of the following treatment arms:
1. GA 20 mg or any IFN-β preparation+oral daily administration of laquinimod capsules 0.6 mg.
2. GA 20 mg or any IFN-β preparation+oral daily administration of laquinimod capsules 1.2 mg.
3. GA 20 mg or any IFN-β preparation+oral daily placebo.

The 0.6 mg laquinimod capsule can be manufactured according to the method disclosed in PCT International Application Publication No. WO/2007/146248, published Dec. 21, 2007 (see, page 10, line 5 to page 11, line 3).

Randomization is stratified in a way that in each arm the number of subjects treated by GA will be equal to the number of subjects treated by IFN-β preparations (Avonex®, Betaseron®/Betaferon®, Rebif® or Extavia®).

During the DBAE phase, subjects continue the same background injectable treatment which they used in the DBPC phase. Subjects who were originally assigned to either of the active oral arms [either laquinimod 0.6 mg (arm 1) or 1.2 mg (arm 2)] continue with their original oral treatment assignment.

Subjects originally assigned to placebo (arm 3) are equally randomized to either laquinimod 0.6 mg or 1.2 mg.

During the DBPC phase, subjects are evaluated at study sites for 11 scheduled visits at Months: −1 (screening), 0 (baseline) and every month thereafter until Month 9 (termination/early termination).

During the DBAE phase subjects are evaluated at study sites for 6 scheduled visits at months 9 [Baseline EXT; the termination visit of the DBPC phase], 10/1AE, 11/2AE, 12/3AE, 1S/4AE and 18/5AE (termination/early termination visit of the DBAE phase).

The following assessments are performed at the specified time points:
1 During both DBPC and DBAE phases, vital signs are measured at each study visit
2 During the DBPC phase, a physical examination is performed at Month −1 (Screening) and Months 0 (Baseline), 1, 3, 6 and 9 (Termination/Early Termination visit of the DBPC phase). During the DBAE phase, a physical examination is performed at Month 9 (Baseline EXT; termination visit of the DBPC phase), 10/1AE, 12/3AE and 18/5AE (Termination/Early Termination visit of the DBAE phase).
3 The following safety clinical laboratory tests are performed:
   a Complete blood count (CBC) with differential—at all scheduled visits in both DBPC and DBAE phases.
   b Serum chemistry (including electrolytes, liver enzymes, creatinine, direct and total bilirubin and pancreatic amylase), and urinalysis is performed at all scheduled visits in both DBPC and DBAE phases. Lipase is tested in case of abnormal pancreatic amylase results. Glomerular filtration rate (GFR) is calculated at Month −1 (Screening) and prior to each MRI scan.
   c Lipid profile (total cholesterol, HDL, LDL and triglycerides) is performed at month −1 (Screening) or Month 0 (Baseline) of the DBPC phase, under fasting conditions.
   d During the DBPC phase, Thyroid function tests (TSH, T3 and free T4) are performed at Months 0 (Baseline), 6 and 9 (Termination/Early termination visit of the DBPC phase). During the DBAE phase thyroid function tests (TSH, T3 and free T4) are performed at Months 9 (Baseline EXT; termination visit of the DBPC phase), 15/4AE and 18/5AE (termination/early termination visit of the DBAE phase).
   e Urinalysis is performed at the Screening visit.
   f Serum β-hCG (human choriogonadotropin beta) is performed in women of child-bearing potential at each scheduled study visit in both DBPC and DBAE phases.
4 Urine dipstick β-hCG in women of child-bearing potential during both the DBPC and the DBAE phases, at all post-Screening study visits and the early termination visit. In addition, during the DBAE Phase, urine β-hCG test is performed at home twice between scheduled visits:
   a. At months 13AE and 14AE (30±4 days and 60±4 after Month 12AE visit, respectively).
   b. At months 16AE and 17AE (30±4 days and 60±4 after Month 15AE visit, respectively).

The subject is contacted by the site staff via telephone within 72 hours after the test is scheduled to be performed and asked specific questions regarding the test. In case of suspected pregnancy (positive urine β-hCG test result), the caller instructs the subject to stop taking the study drug and to arrive to the site as soon as possible (but within 10 days) with all study drugs

During the DBPC phase, electrocardiograms (ECG) is performed at months −1 (screening), 0 (Baseline; three recordings 10 min apart, before first dose), 1, 2, 3, 6 and 9 (termination/early termination visit of the DBPC phase). During the DBAE phase, ECGs are performed at Months 9 (Baseline EXT; termination visit of the DBPC phase), 10/1AE, 11/2AE, 12/3AE, 15/4AE and 18/5AE (Termination/Early Termination visit of the DBAE phase).
6 Chest X-ray is performed at months −1 (screening), if not performed within 6 months prior to the screening visit.
7 Adverse Events (AEs) are monitored throughout the study.
8 Concomitant Medications are monitored throughout the study (both phases).
9 During the DBPC phase, neurological evaluations, including Expanded Disability Status Scale (EDSS), Ambulation Index (AI) and Functional system score (FS) are performed at Months: −1 (screening), 0 (baseline), 3, 6, and 9 (Termination/Early Termination of the DBPC phase). During the DBAE phase, neurological evaluations, including EDSS, AI and FS scores are performed at Months 9 (Baseline; termination visit of the DBPC phase), 12/3AE, 15/4AE and 18/5AE (Termination/Early Termination of the DBAE phase).
10 During the DBPC phase, Symbol Digit Modalities Test (SDMT) are performed at Months 0 (Baseline), 6 and 9 (Termination/Early Termination visit of the DBPC phase). During the DBAE phase, SDMT is performed at Months 9 (Baseline EXT; Termination visit of the DBPC phase), 15/4AE and 18/5AE (Termination/Early Termination visit of the DBAE phase).
11 During the DBPC phase, each subject undergoes 3 MRI scans at Months: 0 (baseline), 3 and 9 (Termination/Early Termination visit of the DBPC phase). During the DBAE phase, each subject undergoes 2 MRI scans at Months 9 (Baseline EXT; Termination visit scan of the DBPC phase) and 18/5AE (Termination/Early Termination visit of the DBAE phase).
12 During the DBPC phase, neurological evaluations, including Expanded Disability Status Scale (EDSS), Ambulation Index (AI) and Functional system score (FS) are performed at Months: −1 (screening), 0 (baseline), 3, 6, and 9 (Termination/Early Termination of the DBPC phase). During the DBAE phase, neurological evaluations, including EDSS, AI and FS scores are performed at Months 9 (Baseline; termination visit of the DBPC phase), 12/3AE, 15/4AE and 18/5AE (Termination/Early Termination of the DBAE phase).
13 During the DBPC phase, Symbol Digit Modalities Test (SDMT) are performed at Months 0 (Baseline), 6 and 9 (Termination/Early Termination visit of the DBPC phase). During the DBAE phase, SDMT is performed at Months 9 (Baseline EXT; Termination visit of the DBPC phase), 15/4AE and 18/5AE (Termination/Early Termination visit of the DBAE phase).
14 During the DBPC phase, each subject undergoes 3 MRI scans at Months: 0 (baseline), 3 and 9 (Termination/Early Termination visit of the DBPC phase). During the DBAE phase, each subject undergoes 2 MRI scans at Months 9 (Baseline EXT; Termination visit scan of the DBPC phase) and 18/5AE (Termination/Early Termination visit of the DBAE phase).
15 Relapses are confirmed/monitored throughout the study (both phases).

Relapse Treatment

The allowed treatment for a relapse is intravenous Methylprednisolone 1 gr/day for up to 5 consecutive days.

Monitoring

The subjects are closely monitored through the study course by an external independent Data Monitoring Committee (DMC).

MRI Activity Alert Criteria

In case 5 or more GdE-T1 lesions are demonstrated on an MRI scan, the MRI reading center issues a notification letter to the Sponsor, investigator and the DMC. MRI parameters of activity are not considered stopping rules and the decision regarding individual subject's participation in the trial is at the discretion of the treating physician.

Ancillary Studies:

Pharmacogenetic (PGx) assessment: Blood samples for PGx parameters are collected from all subjects that signed the informed consent form (separate from that of the core study), pending Ethics Committees approval, during the DBPC phase, preferably at Month 0 (Baseline) or any other visit following Month 0.

Number of Subjects

Approximately 600 subjects.

Inclusion/Exclusion Criteria

Inclusion Criteria
1. Subjects must have a documented MS diagnosis as defined by the Revised McDonald criteria [Ann Neurol 2011: 69:292-302], with a relapsing disease course.
2. Subjects must be relapse free, in a stable neurological condition and free of corticosteroid treatment [intravenous (IV), intramuscular (IM) and/or oral] 60 days prior to randomization.
3. Subjects must be treated with GA (Copaxone®) or an IFN-j preparation (Avonex®, Betaseron®/Betaferon®, Rebif® or Extavia®), at a stable dose for at least 6 months prior to randomization (switching between IFN-β preparations during the 6 months prior to randomization is allowed, switching between any IFN-β preparation and GA, or vice versa, is exclusionary), and there is no plan to change the subject's injectable treatment (either Copaxone® or IFN-β preparation) during the course of the study.
4. Subjects must have an EDSS score of 1.5-4.5 (inclusive) at randomization.
5. Subjects must be between 18 and 55 years of age, inclusive.
6. Women of child-bearing potential must practice an acceptable method of birth control. Acceptable methods of birth control in this study include: surgical sterilization, intrauterine devices, oral contraceptive, contraceptive patch, long-acting injectable contraceptive, partner's vasectomy or double-barrier method (condom or diaphragm with spermicide).
7. Subjects must be able to sign and date a written informed consent prior to entering the study.
8. Subjects must be willing and able to comply with the protocol requirements for the duration of the study.

Exclusion Criteria
1. Have a non-relapsing, progressive form of MS (e.g., PPMS) (as defined by Lublin and Reingold, 1996).
2. An onset of a relapse, unstable neurological condition or any treatment with corticosteroids [intravenous (iv), intramuscular (im) and/or per os (po)] or Adrenocorticotropic hormone 60 days prior to randomization (last day of steroid treatment should be equal or greater than 60 days prior to randomization).
3. Use of experimental or investigational drugs, and/or participation in drug clinical studies within the 6 months prior to randomization.
4. Use of immunosuppressive agents within 6 months prior to randomization.
5. Use of natalizumab (Tysabri®), fingolimod (Gilenya®) or anti-B cell therapy within the 2 years prior to randomization.
6. Previous use of any of the following: cytotoxic agents, Mitoxantrone (Novantrone®), cladribine, laquinimod, total body irradiation, total lymphoid irradiation, stem cell treatment, autologous bone marrow transplantation or allogenic bone marrow transplantation.
7. Previous treatment with intravenous immunoglobulin (IVIG) or plasmapheresis within 2 months prior to randomization.
8. Use of moderate/strong inhibitors of CYP3A4 within 2 weeks prior to the randomization
9. Use of inducers of CYP3A4 within 2 weeks prior to randomization.
10. Pregnancy or breastfeeding.
11. A ≧2×ULN serum elevation of either alanine transaminase (ALT) or aspartate transaminase (AST) at screening.
12. Serum direct bilirubin which is ≧2×ULN at screening
13. Subjects with a potentially clinically significant or unstable medical or surgical condition that would preclude safe and complete study participation, as determined by medical history, physical examinations, ECG, laboratory tests or chest X-ray. Such conditions may include:
   a. A cardiovascular or pulmonary disorder that cannot be well-controlled by standard treatment permitted by the study protocol.
   b. Renal diseases.
   c. Any form of acute or chronic liver disease.
   d. Known human immunodeficiency virus (HIV) positive status.
   e. A history of drug and/or alcohol abuse.
   f. An unstable psychiatric disorder.
   g. Any malignancies, excluding basal cell carcinoma (BCC), in the last 5 years.
14. A glomerular filtration rate (GFR) less than 60 ml/min at screening visit.
15. A known history of sensitivity to gadolinium (Gd).
16. Inability to successfully undergo MRI scanning.
17. Previous endovascular treatment for Chronic Cerebrospinal Venous Insufficiency (CCSVI).
18. Known drug hypersensitivity that would preclude administration of laquinimod, such as hypersensitivity to: mannitol, meglumine or sodium stearyl fumarate.

Route and Dosage Form
1. GA 20 mg or any preparation of interferon-beta (IFN-β)+oral daily administration of laquinimod capsules 0.6 mg (one laquinimod capsule 0.6 mg and one placebo capsule for laquinimod) (applicable to both DBPC and DBAE phases).
2. GA 20 mg/1 mL or an IFN-β preparation+oral daily administration of laquinimod 1.2 mg (2 capsules of laquinimod 0.6 mg) (applicable to both DBPC and DBAE phases)
3. GA 20 mg or an preparation of IFN-β+oral daily administration of placebo (2 placebo capsules for laquinimod) (applicable only to DBPC phase)

Outcome Measures

The primary objectives of the study are to assess the safety, tolerability and efficacy of two daily doses of oral laquinimod (0.6 mg or 1.2 mg) in adjunct to GA or IFN-β preparation (Avonex®, Betaseron®/Betaferon®, Rebif® or Extavia®) in subjects with RMS.

Primary Efficacy Endoint for DBPC Phase:
The percent brain volume change (PBVC) between month 0 (Baseline) to Month 9 (Termination/Early Termination after Month 6 of the DBPC phase).

Key Exploratory Efficacy Endpoints for DBPC Phase:
Change in whole brain Magnetic Transfer Ratio (MTR) histogram between month 0 (Baseline) and Month 9 (Termination/Early Termination visit after Month 6 of the DBPC phase).
Time to Confirmed Disease Progression (CDP). CDP is defined as a sustained increase in EDSS of ≧1 point from Baseline for at least 3 months. Progression cannot be confirmed during a relapse.

Exploratory Endpoints for DBPC Phase
The percent change in cortical thickness between month 0 (baseline) and month 9 (termination/early termination visit after month 6).
The cumulative number new T1 hypointense lesions at months 3 and 9 (termination/early termination visit after month 6).
The number of active (new T2 or GdE-T1) lesions at month 3 that evolved into black holes at month 9 (termination/early termination visit after month 6).
The cumulative number of GdE-T1 lesions at months 3 and 9 (termination/early termination visit after month 6).
Change in T2 lesion volume from 0 (baseline) to month 9 (termination/early termination visit after month 6).
Change in GdE-T1 lesions volume from month 0 (baseline) to month 9 (termination/early termination visit after month 6).
Change from baseline to month 9 (termination/early termination visit after month 6) in SDMT score.
The general health status, as assessed by the EuroQoL (EQ5D) questionnaire.
Assessment of the effect of general health and symptom severity on work, using the work productivity and activities impairment General Health (WPAI-GH) questionnaire.
Annualized Relapse Rate (ARR).
The time to the first confirmed relapse.
Pharmacokinetics of laquinimod.

Exploratory Endpoints for DBAE Phase

A similar set of endpoints we analyzed for the DBAE phase.

Safety and Tolerability Endpoints for DPBC Phase
The cumulative number of GdE-T1 lesions at months 3 and 9.
The cumulative number of Combined Unique Active (CUA) lesions at months 3 and 9.
Number of subjects with adverse events.
Number of subjects with potentially clinically significant abnormalities based on laboratory tests and vital signs and ECGs during the study.
Proportion of subjects (%) who prematurely discontinue from the study, reason of discontinuation and the time to withdrawal.
Proportion of subjects (%) who prematurely discontinue from the study due to adverse events (AEs) and the time to withdrawal.

Results/Discussion

This study assesses safety, tolerability and efficacy of laquinimod in adjunct to glatiramer acetate (GA) or interferon-beta (IFN-β) in relapsing multiple sclerosis (RMS) subjects. Since the mechanisms of action of laquinimod and GA have not been fully elucidated, the effect of the combined therapy cannot be predicted and must be evaluated experimentally.

Daily administration of laquinimod (p.o., 0.6 mg/day and 1.2 mg/day) as an add-on therapy for a patient already receiving glatiramer acetate (GA) (s.c., 20 mg/day) provides increased efficacy (provides an additive effect or more than an additive effect) in relapsing multiple sclerosis (RMS) subjects without unduly increasing adverse side effects or affecting the safety of the treatment.

Daily administration of laquinimod (p.o., 0.6 mg/day and 1.2 mg/day) as an add-on therapy to glatiramer acetate (GA) (s.c., 20 mg/day) is also safe for use in treating relapsing multiple sclerosis (RMS) patients.

Administration of laquinimod (p.o., 0.6 mg/day and 1.2 mg/day) as an add-on therapy to glatiramer acetate (GA) (s.c., 20 mg/day) provides a clinically meaningful advantage and is more effective (provides an additive effect or more than an additive effect) in treating relapsing multiple sclerosis (RMS) patients than when GA is administered alone (at the same dose) in the following manner:
1. The add-on therapy is more effective (provides an additive effect or more than an additive effect) in reducing the decrease in brain volume (determined by the percent brain volume change (PBVC)), in relapsing multiple sclerosis (RMS) patients.
2. The add-on therapy is more effective (provides an additive effect or more than an additive effect) in increasing the time to confirmed disease progression (CDP), in relapsing multiple sclerosis (RMS) patients, where CDP is defined as a sustained increase in EDSS of ≧1 point from Baseline for at least 3 months. Progression cannot be confirmed during a relapse.
3. The add-on therapy is more effective (provides an additive effect or more than an additive effect) in reducing abnormalities observed in whole Brain MTR histogram, in relapsing multiple sclerosis (RMS) patients during.
4. The add-on therapy is more effective (provides an additive effect or more than an additive effect) in reducing the number of confirmed relapses and therefore the relapse rate, in relapsing multiple sclerosis (RMS) patients.
5. The add-on therapy is also more effective (provides an additive effect or more than an additive effect) in reducing the accumulation of physical disability in relapsing multiple sclerosis (RMS) patients, as measured by the time to confirmed progression of EDSS.
6. The add-on therapy is more effective (provides an additive effect or more than an additive effect) in reducing MRI-monitored disease activity in relapsing multiple sclerosis (RMS) patients, as measured by the cumulative number of T1 Gd-enhancing lesions on T1-weighted images, the cumulative number new T1 hypointense lesions, the cumulative number of new T2 lesions, the cumulative number of new T1 hypointense lesions on T1-weight images (black holes), the number of active (new T2 or GdE-T1) lesions, presence or absence of GdE lesions, change in total volume of T1 Gd-enhancing lesions, change in total volume of T2 lesions, and/or cortical thickness.
7. The add-on therapy is more effective (provides an additive effect or more than an additive effect) in reducing brain atrophy in relapsing multiple sclerosis (RMS) patients.
8. The add-on therapy is more effective (provides an additive effect or more than an additive effect) in reducing the frequency of relapses, the frequency of clinical exacerbation, and the risk for confirmed progression in relapsing multiple sclerosis (RMS) patients.
9. The add-on therapy is more effective (provides an additive effect or more than an additive effect) in increasing the time to confirmed relapse in relapsing multiple sclerosis (RMS) patients.
10. The add-on therapy is more effective (provides an additive effect or more than an additive effect) in improving the general health status (as assessed by the EuroQoL (EQ5D) questionnaire), symptom severity on work (as assessed by the work productivity and activities impairment General Health (WPAI-GH) questionnaire) and quality of life, in relapsing multiple sclerosis (RMS) patients.
11. The add-on therapy is more effective (provides an additive effect or more than an additive effect) in decreasing cerebral dysfunction/cognitive impairment (as assessed by Symbol Digit Modalities Test (SDMT)), in relapsing multiple sclerosis (RMS) patients during the double blind study period.

Administration of laquinimod (p.o., 0.6 mg/day and 1.2 mg/day) as an add-on therapy to glatiramer acetate (GA) (s.c., 20 mg/day) provides a clinically meaningful advantage and is more effective (provides an additive effect or more than an additive effect) in delaying the conversion to clinically definite MS in patients presenting a CIS suggestive of MS than when GA is administered alone (at the same dose).

Administration of laquinimod (p.o., 0.6 mg/day and 1.2 mg/day) as an add-on therapy to glatiramer acetate (GA) (s.c., 20 mg/day) provides a clinically meaningful advantage and is more effective (provides an additive effect or more than an additive effect) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons than when GA is administered alone (at the same dose).

Based on the foregoing, analogous results are expected for therapy using laquinimod (p.o., 0.6 mg/day and 1.2 mg/day) in combination with glatiramer acetate (GA) (s.c., 20 mg/day). Specifically, daily administration of laquinimod (p.o., 0.6 mg/day and 1.2 mg/day) in combination with glatiramer acetate (GA) (s.c., 20 mg/day) provides increased efficacy (provides an additive effect or more than an additive effect) over the administration of each agent alone in relapsing multiple sclerosis (RMS) subjects without unduly increasing adverse side effects or affecting the safety of the treatment.

Daily administration of laquinimod (p.o., 0.6 mg/day) in combination with glatiramer acetate (GA) (s.c., 20 mg/day) is also safe for use in treating relapsing multiple sclerosis (RMS) patients.

Administration of laquinimod (p.o., 0.6 mg/day) in combination with glatiramer acetate (GA) (s.c., 20 mg/day) provides a clinically meaningful advantage and is more effective (provides an additive effect or more than an additive effect) in treating relapsing multiple sclerosis (RMS) patients than when each agent is administered alone (at the same dose) in the following manner:

Administration of laquinimod (p.o., 0.6 mg/day and 1.2 mg/day) in combination with glatiramer acetate provides a clinically meaningful advantage and is more effective (provides an additive effect or more than an additive effect) in treating relapsing multiple sclerosis (RMS) patients than when glatiramer acetate is administered alone (at the same dose) in the following manner:
12. The combination therapy is more effective (provides an additive effect or more than an additive effect) in reducing the decrease in brain volume (determined by the percent brain volume change (PBVC)), in relapsing multiple sclerosis (RMS) patients.
13. The combination therapy is more effective (provides an additive effect or more than an additive effect) in increasing the time to confirmed disease progression (CDP), in relapsing multiple sclerosis (RMS) patients, where CDP is defined as a sustained increase in EDSS of ≧1 point from Baseline for at least 3 months. Progression cannot be confirmed during a relapse.
14. The combination therapy is more effective (provides an additive effect or more than an additive effect) in reducing abnormalities observed in whole Brain MTR histogram, in relapsing multiple sclerosis (RMS) patients during.
15. The combination therapy is more effective (provides an additive effect or more than an additive effect) in reducing the number of confirmed relapses and therefore the relapse rate, in relapsing multiple sclerosis (RMS) patients.
16. The combination therapy is also more effective (provides an additive effect or more than an additive effect) in reducing the accumulation of physical disability in relapsing multiple sclerosis (RMS) patients, as measured by the time to confirmed progression of EDSS.
17. The combination therapy is more effective (provides an additive effect or more than an additive effect) in reducing MRI-monitored disease activity in relapsing multiple sclerosis (RMS) patients, as measured by the cumulative number of T1 Gd-enhancing lesions on T1-weighted images, the cumulative number new T1 hypointense lesions, the cumulative number of new T2 lesions, the cumulative number of new T1 hypointense lesions on T1-weight images (black holes), the number of active (new T2 or GdE-T1) lesions, presence or absence of GdE lesions, change in total volume of T1 Gd-enhancing lesions, change in total volume of T2 lesions, and/or cortical thickness.
18. The combination therapy is more effective (provides an additive effect or more than an additive effect) in reducing brain atrophy in relapsing multiple sclerosis (RMS) patients.
19. The combination therapy is more effective (provides an additive effect or more than an additive effect) in reducing the frequency of relapses, the frequency of clinical exacerbation, and the risk for confirmed progression in relapsing multiple sclerosis (RMS) patients.
20. The combination therapy is more effective (provides an additive effect or more than an additive effect) in increasing the time to confirmed relapse in relapsing multiple sclerosis (RMS) patients.
21. The combination therapy is more effective (provides an additive effect or more than an additive effect) in improving the general health status (as assessed by the EuroQoL (EQSD) questionnaire), symptom severity on work (as assessed by the work productivity and activities impairment General Health (WPAI-GH) questionnaire) and quality of life, in relapsing multiple sclerosis (RMS) patients.
22. The combination therapy is more effective (provides an additive effect or more than an additive effect) in decreasing cerebral dysfunction/cognitive impairment (as assessed by Symbol Digit Modalities Test (SDMT)), in relapsing multiple sclerosis (RMS) patients during the double blind study period.

Administration of laquinimod (p.o., 0.6 mg/day and 1.2 mg/day) in combination with glatiramer acetate provides a clinically meaningful advantage and is more effective (provides an additive effect or more than an additive effect) in delaying the conversion to clinically definite MS in patients presenting a CIS suggestive of MS than when glatiramer acetate is administered alone (at the same dose).

Administration of laquinimod (p.o., 0.6 mg/day and 1.2 mg/day) in combination with glatiramer acetate provides a clinically meaningful advantage and is more effective (provides an additive effect or more than an additive effect) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons than when glatiramer acetate is administered alone (at the same dose).

### REFERENCES

1. “COPAXONE®” in Physician's Desk Reference, Thompson Reuters—Physician's Desk Reference Inc., Montvale, N.J., 2008, 3110-3113.
2. Alejandro Horga; Xavier Montalban Jun. 4, 2008; Expert Rev Neurother. 2008; 8(5):699-714.
3. Barkhoft F. (1999) “MRI in Multiple Sclerosis: Correlation with Expanded Disability Status Scale (EDSS)”, Multiple Sclerosis. 5(4):283-286 (Abstract).
4. Bjartmar C, Fox R I. (2002) “Pathological mechanisms and disease progression of multiple sclerosis: therapeutic implication”, Drugs of Today. 38:7-29.
5. Brex et al., (2002) “A longitudinal study of abnormalities on MRI and disability from multiple sclerosis”, N Eng J Med. Jan. 17, 2002 346(3):158-64.
6. Brod et al. (2000) Annals of Neurology. 47:127-131.
7. Brück (2011) “Insight into the mechanism of laquinimod action.” J Neurol Sci. 2011 Jul. 15; 306(1-2):173-9.
8. Brunmark C et al., (2002) “The new orally active immunoregulator laquinimod (ABR-215062) effectively inhibits development and relapses of experimental autoimmune encephalomyelitis”, J Neuroimmunology. 130:163-172.
9. Chabot and Yong, Interferon-1b increases IL-10 in a model of T cell—microglia interaction: Relevance to MS, Neurol. 2000, 55: 1497-1505.
10. Chabot et al., Cytokine production in T lymphocyte-microglia interaction is attenuated by glatiramer acetate: A mechanism for therapeutic efficacy in multiple sclerosis, Mult. Scler., in press.
11. Clinical Trials Website, article entitled “Study of Fampridine-ER Tablets in Patients With Multiple Sclerosis”, retrieved Jul. 10, 2012, <http://clinicaltrials.gov/ct2/show?term=fampridine&cond=multiple+sclersis&phase=2&rank=7>.
12. Comi et al. (2007) LAQ/5062 Study Group. “The Effect of Two Doses of Laquinimod on MRI-Monitored Disease Activity in Patients with Relapsing-Remitting Multiple Sclerosis: A Multi-Center, Randomized, Double-Blind, Placebo-Controlled Study”, Presented at: 52th Annual Meeting of the American Academy of Neurology; Apr. 28-May 5, 2007; Boston, Mass.
13. Compston, Genetic susceptibility to multiple sclerosis, in McAlpine's Multiple Sclerosis, Matthews, B. ed., London: Churchill Livingstone, 1991, 301-319.
14. Conway and Cohen (2010) “Combination therapy in multiple sclerosis”, Lancet Neuorol, 9:299-308.
15. Costello et al. (2007) “Combination therapies for multiple sclerosis: scientific rationale, clinical trials, and clinical practice”, Current Opinion in Neurology, 20:281-285.
16. Dal Canto et al. (1977) Multiple sclerosis. Animal model: Theiler's virus infection in mice. Am. J. Path. 88:497-500.
17. De Stefano et al. (1999) “Evidence of early axonal damage in patients with multiple sclerosis”, Neurology. 52(Suppl 2):A378.
18. Dunitz, M. Multiple sclerosis therapeutics, Ed. Rudick and Goodkin. London: Taylor & Francis, 1999.
19. Durelli et al. and the Independent Comparison of Interferon (INCOMIN) Trial Study Group. (2002) “Every-other-day interferon beta-1b versus once-weekly interferon beta-1a for multiple sclerosis: results of a 2-year prospective randomised multicentre study (INCOMIN)”, Lancet. 359:1453-60.
20. EMEA Guideline on Clinical Investigation of Medicinal Products for the Treatment of Multiple Sclerosis (CPMP/EWP/561/98 Rev. i, November 2006).
21. EPAR, Rebif®, Scientific Discussion.
22. Fernández (2007) “Combination therapy in multiple sclerosis”, Journal of the neurological sciences, 259:95-103.
23. Filippi et al., Glatiramer acetate reduces the proportion of MS lesions evolving into black holes, Neurol., 2001, 57:731-733.
24. Fischer et al., (1999) “The Multiple Sclerosis Functional Composite measure (MSFC): an integrated approach to MS clinical outcome assessment” Multiple Sclerosis, 5(4):244-250.
25. Fisk et al., (1994) “Measuring the Functional Impact of Fatigue: Initial Validation of Fatigue Impact Scale”, Clin Inf Dis. 18 Suppl 1:S79-83.
26. Fisk et al., (1994) “The Impact of Fatigue on Patients with Multiple Sclerosis”, Can J Neurol Sci. 21:9-14.
27. Frenández (2007) “Combination therapy in multiple sclerosis”, Journal of the neurological sciences, 259:95-103.
28. Frohman et al., (2003) “The utility of MRI in suspected MS: report of the Therapeutics and Technology Assessment Subcommittee of the American Academy of Neurology”, Neurology, Sep. 9, 2003, 61(5):602-11.
29. Gold (2008) “Combination therapies in multiple sclerosis”, J Neurol. 255[Suppl 1]:51-60.
30. Golder W, (2007) “Magnetic resonance spectroscopy in clinical oncology”, Onkologie. 27(3): 304-9.
31. Grossman et al., (1994) Magnetization transfer theory and clinical applications in neuroradiology”, RadioGraphics 14:279-290.
32. Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. for Biologics Eval. and Res., Clin./Pharm., Nov. 1999<http//www.fda.gov/cber/gdlns/metabol.pdt>.
33. Gurevich et al. (2010) “Laquinimod suppress antigen presentation in relapsing-remitting multiple sclerosis: in-vitro high-throughput gene expression study” (J Neuroimmunol. 2010 Apr. 15; 221(1-2):87-94. Epub 2010 Mar. 27.
34. Guyatt et al. (1985) “The 6-minute walk a new measure of exercise capacity in patients with chronic heart failure”, Can Med Assoc J., 132:919-823.
35. Hafler and Weiner, M S: A CNS and systemic autoimmune disease, Immunol. Today, 1989, 10:104-107.
36. Hartung et al. (2005) “Significance of neutralizing antibodies to interferon beta during treatment of multiple sclerosis: expert opinions based on the Proceedings of an International Consensus Conference”, Eur J Neurol. 12:588-601.
37. Hauser et al. (1983) “Intensive immunosuppression in progressive multiple sclerosis”, New Engl J Med. 308:173-180.
38. Hla T, Lee M J, Ancellin N, Paik J H, Kluk M J (2001). “Lysophospholipids-receptor revelations”. Science 294 (5548): 1875-8.
39. Hohlfeld et al. (2000) “The neuroprotective effect of inflammation implications for the therapy of multiple sclerosis”, J Neuroimmunol. 107:161-166.
40. Johnson at al., Copolymer 1 reduces relapse rate and improves disability in relapsing-remitting multiple sclerosis: results of a phase III multicenter, double-blind placebo-controlled trial. The Copolymer 1 Multiple Sclerosis Study Group, Neurol., 1995, 45:1268.
41. Kleinschmidt-DeMasters et al. (2005) New England Journal of Medicine 353:369-379.
42. Kurtzke J F, (1983) “Rating neurologic impairment in multiple sclerosis; an expanded disability status scale (EDSS)”, Neurology 33(11): 1444-1452.
43. Lampert, Autoimmune and virus-induced demyelinating diseases. A review, Am. J. Path., 1978, 91:176-208.
44. Langer-Gould et al. (2005) New England Journal of Medicine, 353:369-379.
45. Lublin F D, Reingold S C (1996) “Defining the clinical course of multiple sclerosis”, Neurol. 46:907-911.
46. Martyn, The epidemiology of multiple sclerosis, in McAlpine's Multiple Sclerosis, Matthews, B., ed., London: Churchill Livingstone, 1991, 3-40.
47. McDonald et al., Recommended diagnostic criteria for multiple sclerosis: guidelines from the International Panel on the diagnosis of multiple sclerosis. Ann. Neurol., 2001, 50:121-127.
48. McDonald, (2001) “Guidelines from the International Panel on the Diagnosis of Multiple Sclerosis” Ann. Neurol. 50:121-127.
49. Mehta et al. (1996) “Magnetization transfer magnetic resonance imaging: a clinical review”, Topics in Magnetic Resonance Imaging 8(4):214-30.
50. Miki et al. (1999) “Relapsing-Remitting Multiple Sclerosis: Longitudinal Analysis of MR Images—Lack of Correlation between Changes in T2 Lesion Volume and Clinical Findings”, Radiology. 213:395-399.
51. Milo and Panitch (2011) “Combination therapy in multiple sclerosis”, Journal of Neuroimmunology, 231(2011):23-31.
52. Moraal et al. (2008) “Subtraction MR Images in a Multiple Sclerosis Multicenter Clinical Trial Setting”, Radiology, 250(2):506-514.
53. Multiple sclerosis: its diagnosis, symptoms, types and stages, 2003 <http://www.albany.net/˜tjc/multiple-sclerosis.html>.
54. National MS Society Website, retrieved Jul. 10, 2012<http://www.nationalmssociety.org/for-professionals/researchers/clinical-study-measures/t25-
55. Neuhaus et al. (2003) “Immunomodulation in multiple sclerosis: from immunosuppression to neuroprotection”, Trends Pharmacol Sci. 24:131-138.
56. Noseworthy et al. (2000) “Multiple sclerosis”, N Engl J Med. 343.938-952.
57. Olsson, Immunology of multiple sclerosis, Curr. Opin. Neurol. Neurosurg., 1992, 5:195-202.
58. Panitch et al. for the EVIDENCE (Evidence of Interferon Dose-response: European North American Comparative Efficacy) Study Group and the University of British Columbia MS/MRI Research Group. (2002) “Randomized comparative study of interferon β-1a treatment regiments in MS”, The EVIDENCE Trial Neurology. 59:1496-1506.
59. Parkman, Graft-versus-host Disease, Ann Rev. Med., 1991, 42: 189-197.
60. PCT International Application Publication No. WO 1998/30227, published Jul. 16, 1998.
61. PCT International Application Publication No. WO 2000/05250, published Feb. 3, 2000.
62. PCT International Application Publication No. WO 2000/18794, published Apr. 6, 2000.
63. PCT International Application Publication No. WO 2003/048735, published Jun. 12, 2003.
64. PCT International Application Publication No. WO 2004/103297, published Dec. 2, 2004.
65. PCT International Application Publication No. WO 2006/016036, published Nov. 2, 2006.
66. PCT International Application Publication No. WO 2006/029393, published Mar. 16, 2006.
67. PCT International Application Publication No. WO 2006/029411, published Mar. 16, 2006.
68. PCT International Application Publication No. WO 2006/083608, published Aug. 10, 2006.
69. PCT International Application Publication No. WO 2006/089164, published Aug. 24, 2006.
70. PCT International Application Publication No. WO 2006/116602, published Nov. 2, 2006.
71. PCT International Application Publication No. WO 2007/047863, published Apr. 26, 2007.
72. PCT International Application Publication No. WO 2007/047863, published Apr. 26, 2007.
73. PCT International Application Publication No. WO 2007/146248, published Dec. 21, 2007, international filing date Jun. 12, 2007.
74. PCT International Application Publication No. WO 2009/070298, published Jun. 4, 2009.
75. PCT International Application Publication No. WO 2011/008274, published Jan. 20, 2011.
76. PCT International Application Publication No. WO 2011/022063, published Feb. 24, 2011.
77. PCT International Application Publication No. WO 2012/051106, published Apr. 19, 2012.
78. Polman et al. (2011) “Diagnostic Criteria for Multiple Sclerosis: 2010 Revisions to the McDonald Criteria”, Ann Neural. 69:292-302.
79. Polman et al., (2005) “Diagnostic criteria for multiple sclerosis: 2005 revisions to the McDonald Criteria”, Annals of Neurology, Volume 58 Issue 6, Pages 840-846.
80. Polman et al., (2005) “Treatment with laquinimod reduces development of active MRI lesions in relapsing MS”, Neurology. 64:987-991.
81. Poser et al. (1983) “New Diagnostic Criteria for Multiple Sclerosis: Guidelines for Research Protocols”, Annals of Neurology, March 1983, 13(3):227-230.
82. Rodriguez et al. (1987) Theiler's murine encephalomyelitis: a model of demyelination and persistence of virus. Crit. Rev. Immunol., 7:325.
83. Rosen Y, (2007) “The Recent advances in magnetic resonance neurospectroscopy”, Neurotherapeutics. 27(3):330-45.
84. RTT News Article dated Apr. 12, 2011, entitled “Teva Pharma, Active Biotech Post Positive Laquinimod Phase 3 ALLEGRO Results”.
85. Rudick et al (2006) New England Journal of Medicine, 354:911-923.
86. Rudick, R. (1999) “Disease-Modifying Drugs for Relapsing-Remitting Multiple Sclerosis and Future Directions for Multiple Sclerosis Therapeutics”, Neurotherapeutics. 56:1079-1084.
87. Runström et al. (2002) “Laquinimod (ABR-215062) a candidate drug for treatment of Multiple Sclerosis inhibits the development of experimental autoimmune encephalomyelitis in IFN-β knock-out mice”, (Abstract), Medicon Valley Academy, Malmoe, Sweden.
88. Runström et al. (2006) “Inhibition of the development of chronic experimental autoimmune encephalomyelitis by laquinimod (ABR-215062) in IFN-β k.o. and wild type mice” Journal of Neuroimmunology., 173(2006):69-78.
89. Salama et al. (2003) Multiple Sclerosis, 928-31.
90. Sandberg-Wollheim et al. (2005) “48-week open safety study with high-dose oral laquinimod in patients”, Mult Scler. 11:S154 (Abstract).
91. Sorenson P S. (2006) “Neuralising antibodies to interferon-β—measurement, clinical relevance, and management”, J Neurol. 253[Suppl 6]:VI/16-VI/22.
92. Teitelbaum et al., Suppression of Experimental Allergic Encephalomyelitis by a Synthetic Polypeptide, Eur. J. Immunol., 1971, 1: 242-248.
93. Teitelbaum et al., Suppression of Experimental Allergic Encephalomyelitis with Basic Polymers, Eur. J. Immunol., 1973, 3: 273-279.
94. Teva Press Release dated Aug. 1, 2011, entitled “Results of Phase Im BRAVO Trial Reinforce Unique Profile of Laquinimod for Multiple Sclerosis Treatment”.
95. The National MS Society (USA), The Disease Modifying Drug Brochure, Oct. 19, 2006.
96. U.S. Patent Application Publication No. 2008-0207526, published Aug. 28, 2008 (Strominger et al.).
97. U.S. Patent Application Publication No. 2010-0322900, published Dec. 23, 2010 (Tarcic et al.).
98. U.S. Patent Application Publication No. 2011-0027219, published Feb. 3, 2011 (Tarcic et al.).
99. U.S. Patent Application Publication No. 2011-0034508, published Feb. 10, 2011 (Liast Hayardeny).
100. U.S. Patent Application Publication No. 2011-0217295, published Sep. 8, 2011 (Haviv and Tarcic).
101. U.S. Patent Application Publication No. 2011-0218179, published Sep. 8, 2011 (Haviv and Tarcic).
102. U.S. Patent Application Publication No. 2011-0218203, published Sep. 8, 2011 (Joel Kaye et al.).
103. U.S. Patent Application Publication No. 2011-0230413, published Sep. 22, 2011 (Suhayl Dhib-Jalbut).
104. U.S. Patent Application Publication No. 2011-0230413, published Sep. 22, 2011 (Suhayl Dhib-Jalbut).
105. U.S. Patent Application Publication No. 2012-0010238, published Jan. 12, 2012 (Fristedt).
106. U.S. Patent Application Publication No. 2012-0010239, published Jan. 12, 2012 (Piryatinsky et al.).
107. U.S. Patent Application Publication No. 2012-0142730, published Jun. 7, 2012 (Tarcic et al.).
108. U.S. Pat. No. 3,849,550, issued Nov. 19, 1974 (Teitelbaum et al).
109. U.S. Pat. No. 5,800,808, issued Sep. 1, 1998 (Konfino et al).
110. U.S. Pat. No. 5,858,964, issued Jan. 12, 1999 (Aharoni et al).
111. U.S. Pat. No. 5,981,589, issued Nov. 9, 1999 (Konfino et al).
112. U.S. Pat. No. 6,048,898, issued Apr. 11, 2000 (Konfino et al).
113. U.S. Pat. No. 6,054,430, issued Apr. 25, 2000 (Konfino et al).
114. U.S. Pat. No. 6,077,851, issued Jun. 20, 2000 (Bjork et al).
115. U.S. Pat. No. 6,214,791, issued Apr. 10, 2001 (Arnon et al).
116. U.S. Pat. No. 6,342,476, issued Jan. 29, 2002 (Konfino et al).
117. U.S. Pat. No. 6,362,161, issued Mar. 26, 2002 (Konfino et al).
118. U.S. Pat. No. 7,566,767, issued Jul. 28, 2009 (Strominger et al.).
119. U.S. Pat. No. 7,589,208, issued Sep. 15, 2009 (Jansson et al).
120. U.S. Pat. No. 7,884,208, issued Feb. 8, 2011 (Frenkel et al.).
121. U.S. Pat. No. 7,989,473, issued Aug. 2, 2011 (Patashnik et al).
122. U.S. Pat. No. 8,008,258, issued Aug. 30, 2011 (Aharoni at al).
123. U.S. Pat. No. 8,178,127, issued May 15, 2012 (Safadi et al.).
124. Vollmer et al. (2008) “Glatiramer acetate after induction therapy with mitoxantrone in relapsing multiple sclerosis” Multiple Sclerosis, 00:1-8.
125. Yang et al., (2004) “Laquinimod (ABR-215062) suppresses the development of experimental autoimmune encephalomyelitis, modulates the Th1/Th2 balance and induces the Th3 cytokine TGF-β in Lewis rats”, J. Neuroimmunol. 156:3-9.
126. Yong (2002) “Differential mechanisms of action of interferon-β and glatiramer acetate in MS” Neurology., 59:1-7.
127. Yong (2002) “Differential mechanisms of action of interferon-β and glatiramer acetate in MS” Neurology, 59:1-7.
128. Zou et al. (2002) “Suppression of experimental autoimmune neuritis by ABR-215062 is associated with altered Th1/Th2 balance and inhibited migration of inflammatory cells into the peripheral nerve tissue”, Neuropharmacology, 42:731.

1-136. (canceled)

137. A package comprising
(a) a first pharmaceutical composition comprising an amount of laquinimod and a pharmaceutically acceptable carrier;
(b) a second pharmaceutical composition comprising an amount of glatiramer acetate and a pharmaceutically acceptable carrier; and
(c) instructions for use of the first and second pharmaceutical compositions together to treat a human patient afflicted with relapsing multiple sclerosis or presenting a clinically isolated syndrome.

138-171. (canceled)

172. The package of claim 137, wherein the amount of laquinimod in the first composition is 0.1-2.5 mg.

173-177. (canceled)

178. The package of claim 137, wherein the amount of laquinimod in the first composition is 0.6 mg.

179-182. (canceled)

183. The package of claim 137, wherein the amount glatiramer acetate in the second composition is 0.1-1000 mg.

184-190. (canceled)

191. The package of claim 183, wherein the amount glatiramer acetate in the second composition is 20 mg.

192-201. (canceled)

202. Laquinimod for use as an add-on therapy or in combination with glatiramer acetate in treating a human patient afflicted with multiple sclerosis or presenting a clinically isolated syndrome.

203. (canceled)

204. A pharmaceutical composition comprising an amount of laquinimod and an amount of glatiramer acetate for use in treating a human patient afflicted with an immune disease, wherein the laquinimod and the glatiramer acetate are administered simultaneously or contemporaneously, and wherein the immune disease is an autoimmune disease, an arthritic condition, a demyelinating disease, an inflammatory disease, multiple sclerosis, relapsing-remitting multiple sclerosis, diabetes mellitus, psoriasis, rheumatoid arthritis, inflammatory bowel disease, Crohn's disease, or systemic lupus erythematosus.

205. (canceled)

206. The pharmaceutical composition of claim 204, wherein the pharmaceutical composition is in the form of an aerosol or inhalable powder.

207. The pharmaceutical composition of claim 204, in liquid form.

208. The pharmaceutical composition of claim 204, in solid form.

209. The pharmaceutical composition of claim 208, in capsule form.

210. The pharmaceutical composition of claim 208, in tablet form.

211. The pharmaceutical composition of claim 210, wherein the tablets are coated with a coating which inhibits oxygen from contacting the core.

212-213. (canceled)

214. The pharmaceutical composition of claim 204, further comprising an alkalinizing agent.

215. (canceled)

216. The pharmaceutical composition of claim 204, further comprising an oxidation reducing agent.

217-219. (canceled)

220. The pharmaceutical composition of claim 204, further comprising a lubricant.

221-228. (canceled)

229. The pharmaceutical composition of claim 204, wherein the amount of laquinimod in the composition is 0.1-2.5 mg.

230-234. (canceled)

235. The pharmaceutical composition of claim 229, wherein the amount of laquinimod in the composition is 0.6 mg.

236-239. (canceled)

240. The pharmaceutical composition of claim 204, wherein the amount glatiramer acetate in the composition is 0.1-1000 mg.

241-247. (canceled)

248. The pharmaceutical composition of claim 240, wherein the amount glatiramer acetate in the composition is 20 mg.

249-260. (canceled)
